SEQUENCE LISTING (50 sequences)

SEQ ID NO: 1 - DNA, 5031 bp, Brassica napus
  source 1..5031: mol_type = genomic DNA; note = BnaAHAE gene
agaactcgagtaatgaaacaagaaataggagtcttcgtaacagaaaatttttatttaaaagttgctgttaccggaaagtaattacttttatttataaagaagcatgtgattttgttgaagaaaaacataaatttacactctttttttttatcaaactgcgacaactttataaaagaaaaaatattcaatttataaagaaataggatttagtgggcctatattgtttgacgcttaaagagtccaaaaatacgcaataatggaagaaagccgaagaagcccgttggataaaagaagatattccaagataaaaatccattggtaatcatttgacatataatctaatgatgatatatatatacatgtaaacttttgatttaacaaatacatgtaaacttgtgtttgtaatacgacagcatatacgataatctagatttttattcttaacaagataatttggttacgtattctcccaagacgtgtcttaatctcaatcagtcttaacgtatggtccatgaataacgcattaaattgttctttggaagaatttattcttcggctaaaagaggttatccgttgtgaagtggactaggcaatgcacccttcatttttgtgttagtagaactaacataaaacgagaacttcagtgaccttcatttcatatatttattaaatattgaacatgtagcagacaatatgaaatataaaactttataacgagataaaatcacctatttacataacttttacaatgtctttttcatgttcggtcaaaatactctaaataaggcttcaaaataacagagaacaaaagtccaaagcactaaaaagcaaagtgctgggaggagacgtaaagggaatgcaaactagggacacaaagataattctcgttacaggaccaagaagacttaaacattattggattaaggtatcagtggcatggaccatcatagaggttatggaatagcaaactcgtgatttttgtgataactgattaaagaagacaagatctatcccccttaaatctcgtcctttcaaatcaaaattactgttgctttacctaatgaaacgaaagcaatctaacaaaaaaaaatccactgttagatcatgttcttccgaccaaccaattttgacccttctcctacatacacgtgcctttacaaacgcgcatggaggctttacgaataaaagatgctcaatcaagttctcatttcaacctagggttgggggacataggggggtttaaggtttataagaaagtttatagatagataacaaatcgtaggccatgtaaaacaaaaacagtaatgaagaagtttctatcaagactctcaaaagtcaatacaaatccagcattacatggccctcgtatatctccatcatgttgaccattcgtagtcttttagttgacttggacagtgaccaaattttatcttttttcatgtgaataattcactaatcattcttattaacttatcaataaaattaacaacatagctatttaaatttgtcgtattaaactattaatttcatcactcacgagttttctgatgaattacatgtaaatagtaaatactgtggtagatttcacatgtatgcatataatagctgactgtttgattctcaattttttgaaaaacatgtctcacacatgtcttgtcacagtgttcgcttccctctttgcttctacaatcctgaacctttaaaactcatgatttcgtttgattcacaagtcacataagcatgagagaaaaaaaaataaaacaatcttactagtaaaaatgctttattgtcttattctcctcctcctcctatgcctctcttcaacgtatctatgcctctcactaaaccacgatgccaccatcctccggcaagccaaactgggcttatctgacccggcccagtcactatcttcctggtccgaaaacgacgtcactccgtgccactggcgcggcatcacctgcgacgccacgtcagctgtcttctccgttaatctctccagcttcatgctcgtcggtcctttcccctccgtcctctgccgtcttccttcgctgtctttcctctctctctacaacaactccatcaacggttcgctctccggcgatgagtttacatcgtgtcggaatctcagccaactcgatttgtcggagaatctcctcgttggttccatcccggagtcacttcctttcacgctgccgaacctccgacacttcgaagtttccgggaacaacttatcagatacgattccagcgagcttcggagagttccgcaagctcgagaagttaaacctcgccggaaacctcctctccggtaccattcccgcctcgctcggaaacgtatccactctgagagaactcaaactcgcttacaatctgttttcaccgggtcgaatcccgagtcaactcggtaatcttacggagcttcgtgttctctggctcgccggctgcaacctcgctggtccggtgcctcaagctctgtcccggttgactcacttggtcagcttggatttgacgatgaatcaactcaccgggtctgttccgagttggatcacggagctgaaaagcatcgagcaaatcgagatattcaacaactcgttctcaggcgtgttaccggaggcgatgggtaacatgacgatgctaaagagtttcgacgcgtcgatgaacaagctgacggggaagattcccgacggcttgactcggttaaacctcgagtcgctcaacctcttcgagaacatgctcgaagggcccttgccggagagcattactcgctccaaaacgttaacggaactaaaactgttcaacaacaaactcacaggggagataccgagtcaactcggggcgagctccccattgcagttcgtggacttttcgtataatcagttttccggcgagatacctgcgaatatatgcggaggagggaagctagagtttctcatgcttatagggaactccttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcgatctcagctctaatcagctctccggcgagattccacgtggaattcgtggttggaagaatctgaacgagctcaatttagctaataaccatctctccggcgaagtccctagagagctcggagacttgccggttcttaactacctcgatctctcaaacaatcaattttccggtgagattccaccggagctccagaatctgaagctcaacgttataaacctatcgtataatcatctctccgggagagtgcctcctctctacgctaacaaaatctacgattctagctttgtcggaaaccatgacttggcgtcgatgatcacgacagtctttgtcggaagaagagcacacggtctcaaaacatcggttatgtctggattctcctatcgattttcatactcgcctgtatggttttcgtcgttggggttgttatgtttatcgccaactgcaagaagatgagagcctccaagagcgctagattctccgcgtctaagtggagatccttccacaagcttcatttcagcgagcatgagatcgttgattgccttgacgaacggaacgtgatcggatctggctcctccggtaaagtctatagagtggagcttagtggcggagaagtagtagtagctgtgaagaaactaaacaaaacggctaaaggaggggaagacgattcgttgaacagagacgttttcgctgcagaggtggaaacgcttggaacgattaggcataagagtattgtgcgcttgtggtgctgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttcctccgatcgtgcaccgtgacgtgaagtctagtaacatattgctagatgcagattatggagccaaagtagctgactttggcattgctaaaatcggtcaaatgagcggtgctaaatcgccagaagctatgtctggaatcgccggttcttgcggttacattgcaccaggtataaccaaatcaaatgataaaaagataacttatatttcaaatgttatttgtgctttatagctcttttttatgctgttttgtagaatacgtatatacacttagggtgaatgaaaagagtgatatctacagttttggtatcgtgcttttggagttggtaacggggaagcaaccaacagatctagaacttggagataaagatatggtgaagtgggtgtgctctacccttgaccaatgcggtttagaatcggtgatcgatcccaaacttgatctggggttcaaagaagagattagcaaagtcattcacattggtcttcttttacgagtcctctccctctaaaccgaccttccatgagaaaagttgtgatcatgcttcaagaagtctctggtgctgtttctagtagcagaccaaatgcgtccaaacgctctaagagcagtgggaaactctcgccttactatgtggaagacgtaaacagcgtttgagcattcaaatatgggccggtatgggttgggctgggccatatgtattaattgggttgtatagttaggtgaattgtaagaaatcagaaccagaaaagtggacctttctgattaggtaatggttttaatgctatgatgtttggatattaaaaatatttttaattaacgcgatgatt

SEQ ID NO: 2 - DNA, 2997 bp, Brassica napus
  source 1..2997: mol_type = other DNA; note = BnaAHAE cDNA
atgctttattgtcttattctcctcctcctcctatgcctctcttcaacgtatctatgcctctcactaaaccacgatgccaccatcctccggcaagccaaactgggcttatctgacccggcccagtcactatcttcctggtccgaaaacgacgtcactccgtgccactggcgcggcatcacctgcgacgccacgtcagctgtcttctccgttaatctctccagcttcatgctcgtcggtcctttcccctccgtcctctgccgtcttccttcgctgtctttcctctctctctacaacaactccatcaacggttcgctctccggcgatgagtttacatcgtgtcggaatctcagccaactcgatttgtcggagaatctcctcgttggttccatcccggagtcacttcctttcacgctgccgaacctccgacacttcgaagtttccgggaacaacttatcagatacgattccagcgagcttcggagagttccgcaagctcgagaagttaaacctcgccggaaacctcctctccggtaccattcccgcctcgctcggaaacgtatccactctgagagaactcaaactcgcttacaatctgttttcaccgggtcgaatcccgagtcaactcggtaatcttacggagcttcgtgttctctggctcgccggctgcaacctcgctggtccggtgcctcaagctctgtcccggttgactcacttggtcagcttggatttgacgatgaatcaactcaccgggtctgttccgagttggatcacggagctgaaaagcatcgagcaaatcgagatattcaacaactcgttctcaggcgtgttaccggaggcgatgggtaacatgacgatgctaaagagtttcgacgcgtcgatgaacaagctgacggggaagattcccgacggcttgactcggttaaacctcgagtcgctcaacctcttcgagaacatgctcgaagggcccttgccggagagcattactcgctccaaaacgttaacggaactaaaactgttcaacaacaaactcacaggggagataccgagtcaactcggggcgagctccccattgcagttcgtggacttttcgtataatcagttttccggcgagatacctgcgaatatatgcggaggagggaagctagagtttctcatgcttatagggaactccttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcgatctcagctctaatcagctctccggcgagattccacgtggaattcgtggttggaagaatctgaacgagctcaatttagctaataaccatctctccggcgaagtccctagagagctcggagacttgccggttcttaactacctcgatctctcaaacaatcaattttccggtgagattccaccggagctccagaatctgaagctcaacgttataaacctatcgtataatcatctctccgggagagtgcctcctctctacgctaacaaaatctacgattctagctttgtcggaaaccatgacttgtgcgtcgatgatcacgacagtctttgtcggaagaagagcacacggtctcaaaacatcggttatgtctggattctcctatcgattttcatactcgcctgtatggttttcgtcgttggggttgttatgtttatcgccaactgcaagaagatgagagcctccaagagcgctagattctccgcgtctaagtggagatccttccacaagcttcatttcagcgagcatgagatcgttgattgccttgacgaacggaacgtgatcggatctggctcctccggtaaagtctatagagtggagcttagtggcggagaagtagtagtagctgtgaagaaactaaacaaaacggctaaaggaggggaagacgattcgttgaacagagacgttttcgctgcagaggtggaaacgcttggaacgattaggcataagagtattgtgcgcttgtggtgctgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttcctccgatcgtgcaccgtgacgtgaagtctagtaacatattgctagatgcagattatggagccaaagtagctgactttggcattgctaaaatcggtcaaatgagcggtgctaaatcgccagaagctatgtctggaatcgccggttcttgcggttacattgcactagaatacgtatatacacttagggtgaatgaaaagagtgatatctacagttttggtatcgtgcttttggagttggtaacggggaagcaaccaacagatctagaacttggagataaagatatggtgaagtgggtgtgctctacccttgaccaatgcggtttagaatcggtgatcgatcccaaacttgatctggggttcaaagaagagattagcaaagtcattcacattggtcttctttgtacgagtcctctccctctaaaccgaccttccatgagaaaagttgtgatcatgcttcaagaagtctctggtgctgtttctagtagcagaccaaatgcgtccaaacgctctaagagcagtgggaaactctcgccttactatgtggaagacgtaaacagcgtttga

SEQ ID NO: 3 - protein, 998 aa, Brassica napus
  source 1..998: note = BnaAHAE aa
MLYCLILLLLLCLSSTYLCLSLNHDATILRQAKLGLSDPAQSLSSWSENDVTPCHWRGITCDATSAVFSVNLSSFMLVGPFPSVLCRLPSLSFLSLYNNSINGSLSGDEFTSCRNLSQLDLSENLLVGSIPESLPFTLPNLRHFEVSGNNLSDTIPASFGEFRKLEKLNLAGNLLSGTIPASLGNVSTLRELKLAYNLFSPGRIPSQLGNLTELRVLWLAGCNLAGPVPQALSRLTHLVSLDLTMNQLTGSVPSWITELKSIEQIEIFNNSFSGVLPEAMGNMTMLKSFDASMNKLTGKIPDGLTRLNLESLNLFENMLEGPLPESITRSKTLTELKLFNNKLTGEIPSQLGASSPLQFVDFSYNQFSGEIPANICGGGKLEFLMLIGNSFSGEIPINLGKCRSLTRVRLNNNKLSGHVPEEFWGLPRMSLLELSENSFTGRISESIAGAKNLSNLRISKNQFSGSIPGEIGSLNGLIEITGDENSFSGEVPSTLVKLKQLSRLDLSSNQLSGEIPRGIRGWKNLNELNLANNHLSGEVPRELGDLPVLNYLDLSNNQFSGEIPPELQNLKLNVINLSYNHLSGRVPPLYANKIYDSSFVGNHDLCVDDHDSLCRKKSTRSQNIGYVWILLSIFILACMVFVVGVVMFIANCKKMRASKSARFSASKWRSFHKLHFSEHEIVDCLDERNVIGSGSSGKVYRVELSGGEVVVAVKKLNKTAKGGEDDSLNRDVFAAEVETLGTIRHKSIVRLWCCCSSGDCKLLVYEYMPNGSLADVLHSHCKGGVLLGWPERLRIGLDAAEGLSYLHHDCVPPIVHRDVKSSNILLDADYGAKVADFGIAKIGQMSGAKSPEAMSGIAGSCGYIALEYVYTLRVNEKSDIYSFGIVLLELVTGKQPTDLELGDKDMVKWVCSTLDQCGLESVIDPKLDLGFKEEISKVIHIGLLCTSPLPLNRPSMRKVVIMLQEVSGAVSSSRPNASKRSKSSGKLSPYYVEDVNSV

SEQ ID NO: 4 - DNA, 5292 bp, Brassica napus
  source 1..5292: mol_type = genomic DNA; note = BnaCHAE gene
aaaacttgagtaatgaaacaagaaattagggtgttgtgttgtcttttgttctgaatttaattaacactaggattggcgtatgacgtctatcttggtttaattcttcatgtttcttaggagttttttaattttttgcggcttctaagaacaaaccagctttcgagcagaatctggttcaactttgttgtaaccaaactttcattcatgaatgatatttacaatttagcaaaaaaaaaaaaaaaaaacttgagtaatgaaacaagaaataggagtcttcgtaacagaaactttttatttaaaagttgctgttaccggaaagaaattacttttatttataataagcatgtgattttgttgaaaaaaaaaacataaattacacattttttttatcaaactgcgacaaatttataaaagaacttcaatttataaagaagtagcatttagtgggcctatattgtttgacgcttaaagagtccaaaaatacgcaataatggaagaaagccgaagaagcccgttggataaaagaagatattccaagataaaatccactggtaatcatttgcatataatctaatgatatacatgtaaacttgtgtttgtaatacgacagcatatacgataatctagattttttgttcttaacaagataatttagctacgtattctccaaatacgtggcttaatctcagtcagtcttaacgtatggtctatgaacaacgcattaaattgttctttggaagaatttattcttcggctaaaagaggttatccgttgtgaagtggactaggcaatgcacccttcacttttgtgttagtagaactaacatgaaacgagaaagaacttcggtgacctccatttcatatatttattacatattgaacatgtagacaatatgaaatataaaacataaaactttataacgagataaaataacgtatttacataacttttacaatgtctttttcatgttcggtcaaaatactctctataaataaggcttcagaataacagagaacaaaagtccaaagcactaaaaagcaaagtgcttgggggagacgtaaagggaatgcaaacaaaggtgtaattatagttacaggaccaagaagacttaaacattattggattaaggtatcagtggtatggaccatcatcgaggttgtagagttgcaaactcgtgatatttgcgataagctgattaaagaagacaagatctatcccccttaaatttcgtcctttcaaatcaaaatgactgttactttacctaacgaaatgatagcaatctaacaaaaagatccactgttagatcatgttcttccgaccaaccaaatttgaccttgtctacatacacgtgcccgcgttaagcctttacaaataaaagatgctcaatcaacttctcatttcaacctagggttgggggacataggggtttaggtttaaggttcttaagaaagtttatagatagtagataacaaatcatagggatataatgtggaatatcaatctaataatcgatggttgtacattaattaaaacaaaaacagtaatgaagaagtttctatcaagactctcaaaagtcgatacaaatccagcattacatggcctacgtatatctccatcatgttgaccattcgtagtcttttagttgacttggacagtgaccaaagtttatcttttttcatgtgaataatttactaatcattcttattaactaatcaataaaattaacaacatagctttttaaatttgtggtattaaaccattaatttcatcactcacgagttttgtgatgaattatatgtaaatagtaaatactgtggtagatttcacatggatgcatataatagctgactgtttgattctcaattttttgaagaacatgtctcacacatgtcttgtcacagtgttcgcttccctctttgcttctacaatcctgaacctttaaaactcatgatttcgttttattcacaagtcataagcatgagagagagagagaaaattaaaacaatctttctagtaaaaatgctttattgtcttattctcctcctcctcctatgcctctcttccacgtatctatgtctctcactaaaccacgatgccaccatcctccggcaagccaaactgggcttatctgacccggcccagtcactatcttcctggtccgaaaacgacgtcactccgtgccactggcgcggcatcacctgcgacgccacctcagctgtcttctccgttaatctctcaagcttcatgctcgtcggtcctttcccttccatcctctgccgtcttccttctctgtcattcctctctctctataacaactccatcaacggttccctctccggcgatgattttgcatcgtgtcggaatctcatccatctcaatttgtcggagaatctcttggttggttccatcccggagtcactccctttcaagctgccgaacctccgccgcttcggaatatccgggaacaacttatcggacacgattccggcgagcttcggagagttccggaagctcgagaagttaaaccttgccggaaacctcctctccggcaccattcccgcctcgctcggcaacgtatccactctgagagaactcagactcgcttacaatatattttctcctggtcgaatcccgagtcaactcggtaatctaacggagcttcgtgttctctggctcgccggctgcaacctcgctggttcggtgcctcaaactctgtcccggttgactcacttggtcagcttggatttgaccatgaatcaactcaccgggtctgtcccgagttggatcacggagctgagaagcatcgagcaaatcgagatattcaacaactcgttctcaggcgagttaccggaggcgatgggtaacatgacgatgctaaagagtttcgacgcgtcgacgaacaagctgacggggaagatccccgagggcttggctcggttaaacctcgagtcgctcaacctcttcgagaacatgctcgaagggcccttgccggagagcataactcgctccaaaacgttaaccgaactcaagctgttcaacaacaaactcaccggggagataccgagtcaactcggcgcgaactctccgttacagtacgtggacttttcgtataaccagttttccggcgagataccggcgaatatatgcggaggagggaagctagagtatctcatgcttatagggaactcgttttccggtaacataccgacaaatcttggaaagtgcaggagcttgacacgtgtcagactaaataacaacaagctttccggtcacgttccggaagagttctggggtttgcctcgtatgtcgctgctcgaactctcggagaactcgttcaccggaagaatctctgagtccgtcgccggtgcaaagaatctatcaaacctgagaatctcaaagaatcagttctcaggttcgattcccggcgaaatcggatcgcttaacggactcatcgagatcgccggagatgaaaatagcttctccggcgaggttactagtactttggtgaagcttaagcaactgagcaggctcgatctcagctctaatcaactctccggcgagattccacgtggaatccgtggctggaagaatctgaacgagctcaatttagctaataaccatctctccggtgaagcccctagagagctcggagacttgtcggttcttaactacctcgatctctcgaacaatcaattttccggtgagattccaccggagctccagaatctgaagctgaacattataaacctatcgtataatcatctcaccgggagagtaccgcctctctacgctaacaaaatctacgcttctagctttgtcggcaaccatgacttgtgcgtagatgatcacgacagtcgttggcggaagatcgcacggtctaaaaacatcggttatgtctggattctcctatctattttcatactcgcctgtatggttttcgtcgttggggttgttatgtttatcgccaactgcaagaagatgagagcatccaagagcggtagattctctgcgtccaagtggagaagcttccacaagcttcatttcagcgagcatgagatcgttgattgccttgacgaacggaacgtgatcggttccggttcctccggtaaagtctacagagtggagcttagtggcggagaagtagtagtagctgtgaagaaactaaacaaaacggctaaagggggtgaagacgattcgttgaacagagacgttttcgctgcggaggtggaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttccaccgattgtccaccgtgacgtgaagtctagtaacctattgctagatgcagattatggcgccaaagtagctgactttggtattgccaaaatcggtcaaatgaccggtactaaatcgccagaagctatgtccggaatcgctggttcttgcggttacattgcaccaggtatgataacataaccaaataaaatgataaaaccatacaagttacataatgtttcaaatgatattggtgcgttaatagttttttctttctaatgtttctgcagaatacgtatatacactcagggtgaatgaaaagagtgatatctacagttttggtatcgtgcttttggagttggtaacggggaagcaaccaacagatctagaacttggagataaagatatggtgaagtgggtgtgcactacccttgaccaaagcggtttagaaacggtgattgatcccaaacttgatcttgggttcaaagaagagattagcaaagtcattcacattggtcttctctgtacgagtcctctccctctaaaccgaccttccatgagaaaagttgtgatcatgcttcaagaagtctctggtgctgtttctattagcggtccaaatgcgtccaaacgctctaagagcagtgggaaactctcgccttactatgtggaagacgtaaacagcgtttgagcattcaaatatgggccggtatgggttgcgctgggccagaaatagttatttggtttgtattaagttaggtgaattgtaagaaatcagaaacagaaaaatggagctttctgattaggtaatagcttttaatgctatgatgtttggatattaaaaatatttttaattaacgcgatgat

SEQ ID NO: 5 - DNA, 2994 bp, Brassica napus
  source 1..2994: mol_type = other DNA; note = BnaCHAE cDNA
atgctttattgtcttattctcctcctcctcctatgcctctcttccacgtatctatgtctctcactaaaccacgatgccaccatcctccggcaagccaaactgggcttatctgacccggcccagtcactatcttcctggtccgaaaacgacgtcactccgtgccactggcgcggcatcacctgcgacgccacctcagctgtcttctccgttaatctctcaagcttcatgctcgtcggtcctttcccttccatcctctgccgtcttccttctctgtcattcctctctctctataacaactccatcaacggttccctctccggcgatgattttgcatcgtgtcggaatctcatccatctcaatttgtcggagaatctcttggttggttccatcccggagtcactccctttcaagctgccgaacctccgccgcttcggaatatccgggaacaacttatcggacacgattccggcgagcttcggagagttccggaagctcgagaagttaaaccttgccggaaacctcctctccggcaccattcccgcctcgctcggcaacgtatccactctgagagaactcagactcgcttacaatatattttctcctggtcgaatcccgagtcaactcggtaatctaacggagcttcgtgttctctggctcgccggctgcaacctcgctggttcggtgcctcaaactctgtcccggttgactcacttggtcagcttggatttgaccatgaatcaactcaccgggtctgtcccgagttggatcacggagctgagaagcatcgagcaaatcgagatattcaacaactcgttctcaggcgagttaccggaggcgatgggtaacatgacgatgctaaagagtttcgacgcgtcgacgaacaagctgacggggaagatccccgagggcttggctcggttaaacctcgagtcgctcaacctcttcgagaacatgctcgaagggcccttgccggagagcataactcgctccaaaacgttaaccgaactcaagctgttcaacaacaaactcaccggggagataccgagtcaactcggcgcgaactctccgttacagtacgtggacttttcgtataaccagttttccggcgagataccggcgaatatatgcggaggagggaagctagagtatctcatgcttatagggaactcgttttccggtaacataccgacaaatcttggaaagtgcaggagcttgacacgtgtcagactaaataacaacaagctttccggtcacgttccggaagagttctggggtttgcctcgtatgtcgctgctcgaactctcggagaactcgttcaccggaagaatctctgagtccgtcgccggtgcaaagaatctatcaaacctgagaatctcaaagaatcagttctcaggttcgattcccggcgaaatcggatcgcttaacggactcatcgagatcgccggagatgaaaatagcttctccggcgaggttactagtactttggtgaagcttaagcaactgagcaggctcgatctcagctctaatcaactctccggcgagattccacgtggaatccgtggctggaagaatctgaacgagctcaatttagctaataaccatctctccggtgaagcccctagagagctcggagacttgtcggttcttaactacctcgatctctcgaacaatcaattttccggtgagattccaccggagctccagaatctgaagctgaacattataaacctatcgtataatcatctcaccgggagagtaccgcctctctacgctaacaaaatctacgcttctagctttgtcggcaaccatgacttgtgcgtagatgatcacgacagtcgttggcggaagatcgcacggtctaaaaacatcggttatgtctggattctcctatctattttcatactcgcctgtatggttttcgtcgttggggttgttatgtttatcgccaactgcaagaagatgagagcatccaagagcggtagattctctgcgtccaagtggagaagcttccacaagcttcatttcagcgagcatgagatcgttgattgccttgacgaacggaacgtgatcggttccggttcctccggtaaagtctacagagtggagcttagtggcggagaagtagtagtagctgtgaagaaactaaacaaaacggctaaagggggtgaagacgattcgttgaacagagacgttttcgctgcggaggtggaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttccaccgattgtccaccgtgacgtgaagtctagtaacctattgctagatgcagattatggcgccaaagtagctgactttggtattgccaaaatcggtcaaatgaccggtactaaatcgccagaagctatgtccggaatcgctggttcttgcggttacattgcaccagaatacgtatatacactcagggtgaatgaaaagagtgatatctacagttttggtatcgtgcttttggagttggtaacggggaagcaaccaacagatctagaacttggagataaagatatggtgaagtgggtgtgcactacccttgaccaaagcggtttagaaacggtgattgatcccaaacttgatcttgggttcaaagaagagattagcaaagtcattcacattggtcttctctgtacgagtcctctccctctaaaccgaccttccatgagaaaagttgtgatcatgcttcaagaagtctctggtgctgtttctattagcggtccaaatgcgtccaaacgctctaagagcagtgggaaactctcgccttactatgtggaagacgtaaacagcgtttga

SEQ ID NO: 6 - protein, 997 aa, Brassica napus
  source 1..997: note = BnaCHAE aa
MLYCLILLLLLCLSSTYLCLSLNHDATILRQAKLGLSDPAQSLSSWSENDVTPCHWRGITCDATSAVFSVNLSSFMLVGPFPSILCRLPSLSFLSLYNNSINGSLSGDDFASCRNLIHLNLSENLLVGSIPESLPFKLPNLRRFGISGNNLSDTIPASFGEFRKLEKLNLAGNLLSGTIPASLGNVSTLRELRLAYNIFSPGRIPSQLGNLTELRVLWLAGCNLAGSVPQTLSRLTHLVSLDLTMNQLTGSVPSWITELRSIEQIEIFNNSFSGELPEAMGNMTMLKSFDASTNKLTGKIPEGLARLNLESLNLFENMLEGPLPESITRSKTLTELKLFNNKLTGEIPSQLGANSPLQYVDFSYNQFSGEIPANICGGGKLEYLMLIGNSFSGNIPTNLGKCRSLTRVRLNNNKLSGHVPEEFWGLPRMSLLELSENSFTGRISESVAGAKNLSNLRISKNQFSGSIPGEIGSLNGLIEIAGDENSFSGEVTSTLVKLKQLSRLDLSSNQLSGEIPRGIRGWKNLNELNLANNHLSGEAPRELGDLSVLNYLDLSNNQFSGEIPPELQNLKLNIINLSYNHLTGRVPPLYANKIYASSFVGNHDLCVDDHDSRWRKIARSKNIGYVWILLSIFILACMVFVVGVVMFIANCKKMRASKSGRFSASKWRSFHKLHFSEHEIVDCLDERNVIGSGSSGKVYRVELSGGEVVVAVKKLNKTAKGGEDDSLNRDVFAAEVETLGTIRHKSIVRLWCCCSSGDCKLLVYEYMPNGSLADVLHSHCKGGVLLGWPERLRIGLDAAEGLSYLHHDCVPPIVHRDVKSSNLLLDADYGAKVADFGIAKIGQMTGTKSPEAMSGIAGSCGYIAPEYVYTLRVNEKSDIYSFGIVLLELVTGKQPTDLELGDKDMVKWVCTTLDQSGLETVIDPKLDLGFKEEISKVIHIGLLCTSPLPLNRPSMRKVVIMLQEVSGAVSISGPNASKRSKSSGKLSPYYVEDVNSV

SEQ ID NO: 7 - DNA, 3198 bp, Brassica napus
  source 1..3198: mol_type = genomic DNA; note = BnaAHSL2 gene
atgaccaccaccattactactacttctctctttttgttacttgttctctcatgtgttctccaagcttcttccaacggagacgctgagatactgagcagagtcaaaaccagcagactttctgacccggatggaaagttaaatgattgggtcataaccggagataaccggagtccctgtaactggactggaatcacatgcgactccaaaaacggcgccgtcacggcaatagatctctccgactacggaatctccggtgggttcccgtacgggttctgccgtatacgtaccctcgtcaacatcactctctctaaaaacaatctcaacggtacgatagattcagctcctctcacactatgctctcgaatccacgtcctgatcctcacagaaaacagcttctccgggaagctaccggagttttcgccggagtttcgcaacctacgcgttctcgaactggagtccaactttttctccggcgagatccccgaaagctacggaaagcttccatctctgcaagttctgaatctcaacggcaactcgctcggcggaatcgttccggcgtttttgggtaacctgaccgagttgactcgcctcgagctcgcgtacgttcagttcgagcctggtccgattccgtcgacgtttgggaacttgacgaagatgacttatctccggctaactaactcgaacatcgtcggagagattcctgattcgatcgggaatctggtttccctggtgaatctcgatttagctcagaacggtctctccggagagataccggagagcatcggaaagctaaaatcgatttatcaaatggtgctttacatcaatcagctatcgggaaaattgccggagagtatcggaaacttaacggctatgaggaatttcgacgtttcgcagaataatctctccggtgatttgcctgagacgatcgccgctctccaggtcgtttctttccatctcaatgataatttattcaccggtgaattgccacgtggcattgctttgaatcctaatctcgttgatttcaagatctttaacaacagtttcaccgggagtttacctacgagtttcggtaaattctccggtttaactgagttcgacgtctcgactaacagattctccggtgagttgcctccgtatctatgctacgggaagaaacttgaaaagctaatcattttcagcaatcagttaagcggcgagattccggaaacgtacggtgagtgcgacacgcttaattatattagaatggcggataacaaactctccggcgaagttccggttaagttttgggagcttcctctcactcgtctcgagctatctaacaaccgattagaaggttcgattcctccgtcgatatccaaagctcgtcagctctctcagctcgagatctccggaaacaagttgtccggtgcgattccggcgagaatctgtgatctcgaaggactcagggacgtcgatctcagccgtaaccgtttctcaggatctattccctcttgcattaaccgattgaagaatctggagagagtggagatgcaggagaacatgctcgacggcgagatcccgagttcggtgagttcatgcgccaagttaacggagttgaatctctccgataaccgtctccgcggcgaaattccaccggagctcggtgagttaccggttttgaactatctggatctctccaacaaccaactctccggtgagatcccggcggagttgttgaagctgaagctgaatctgttcaacgtctccgataacaaactctccgggaagataccttctggttttcaacaagatgtttttctaccgagttttttgggtaacccgggactctgtgctccggatatggatccgattcgaccttgccgatccaaacccgagccccggtttatcctggttatctccgtagtctgcatcgttgtactaatcggagctttggtttggctttttattaaaaccaaaccgttattccagagaaaaccgaaccggaccgataaagtaaccattttccagcggatcgggttcactgaggaagacatatacccgcaattaacagatgataacataatcgggtcgggcgggtcgggtttggtttacagagtgacactaaagtcaggccaaacgctcgcggtgaagaaactttggggtggaccgggtcaaaaacccgaatccgaatctgttttcagatctgaggtagagattctgggtcgggtcagacatggtaacatagtgaagcttctcatgtgttgcagcggcgaggagtttcggtttttagtgtacgagtatatggaaaacggcagcttgggcgacgttttgcattcggagaaagaacatcgtgccgtttctccgcttgattggacgacacggttttcgatcgcgctgggtgcagctcaaggacttgcttacctacatcatgactctgttccgccgattgttcaccgcgacgtcaaaagcaataatatattgttggaccatgagatgaagccacgtgttgctgatttcggtttagctaaaccgttgagaagagaagtcaataatggtgtctcggatgtttctcctatgtcttgtgttgctggatcctacggctacattgctccaggttcgaattctaaatgctaaaccgaaataaaccggataaccaaatatttatgaatgttatttttattctgttggttgtgattgcacatgcagaatatggttatacgtcgagagtgaatgagaagagcgatgtgtatagttttggtgtcgtattactcgagttgattacgggtaaaagaccaaatgattcgtcgtttggggagaataaagacattgttaagtttgcaatggagtcagctttgtcttactcttctccatcacctgaagacaaagctatgactcaagattcacctggaaattgtcgagatcttagtaagcttgtggatccaaagatggaactttctaggggggagtatgaagaggtagataaagtttttgagattgcgttgctttgcacgtcttcgtttccgattagtaggccgaccatgaggaaagtagtagagctgctcaaagagaagaagccacttgagtgagacggtgttcgttttcttcatctggttttaatactaagattgttacttagtcataggtgggctattggaaaactaattttaaaaacttacatagctagttatcttttttttttgttaatttaaagattaatta

SEQ ID NO: 8 - DNA, 2970 bp, Brassica napus
  source 1..2970: mol_type = other DNA; note = BnaAHSL2 cDNA
atgaccaccaccattactactacttctctctttttgttacttgttctctcatgtgttctccaagcttcttccaacggagacgctgagatactgagcagagtcaaaaccagcagactttctgacccggatggaaagttaaatgattgggtcataaccggagataaccggagtccctgtaactggactggaatcacatgcgactccaaaaacggcgccgtcacggcaatagatctctccgactacggaatctccggtgggttcccgtacgggttctgccgtatacgtaccctcgtcaacatcactctctctaaaaacaatctcaacggtacgatagattcagctcctctcacactatgctctcgaatccacgtcctgatcctcacagaaaacagcttctccgggaagctaccggagttttcgccggagtttcgcaacctacgcgttctcgaactggagtccaactttttctccggcgagatccccgaaagctacggaaagcttccatctctgcaagttctgaatctcaacggcaactcgctcggcggaatcgttccggcgtttttgggtaacctgaccgagttgactcgcctcgagctcgcgtacgttcagttcgagcctggtccgattccgtcgacgtttgggaacttgacgaagatgacttatctccggctaactaactcgaacatcgtcggagagattcctgattcgatcgggaatctggtttccctggtgaatctcgatttagctcagaacggtctctccggagagataccggagagcatcggaaagctaaaatcgatttatcaaatggtgctttacatcaatcagctatcgggaaaattgccggagagtatcggaaacttaacggctatgaggaatttcgacgtttcgcagaataatctctccggtgatttgcctgagacgatcgccgctctccaggtcgtttctttccatctcaatgataatttattcaccggtgaattgccacgtggcattgctttgaatcctaatctcgttgatttcaagatctttaacaacagtttcaccgggagtttacctacgagtttcggtaaattctccggtttaactgagttcgacgtctcgactaacagattctccggtgagttgcctccgtatctatgctacgggaagaaacttgaaaagctaatcattttcagcaatcagttaagcggcgagattccggaaacgtacggtgagtgcgacacgcttaattatattagaatggcggataacaaactctccggcgaagttccggttaagttttgggagcttcctctcactcgtctcgagctatctaacaaccgattagaaggttcgattcctccgtcgatatccaaagctcgtcagctctctcagctcgagatctccggaaacaagttgtccggtgcgattccggcgagaatctgtgatctcgaaggactcagggacgtcgatctcagccgtaaccgtttctcaggatctattccctcttgcattaaccgattgaagaatctggagagagtggagatgcaggagaacatgctcgacggcgagatcccgagttcggtgagttcatgcgccaagttaacggagttgaatctctccgataaccgtctccgcggcgaaattccaccggagctcggtgagttaccggttttgaactatctggatctctccaacaaccaactctccggtgagatcccggcggagttgttgaagctgaagctgaatctgttcaacgtctccgataacaaactctccgggaagataccttctggttttcaacaagatgtttttctaccgagttttttgggtaacccgggactctgtgctccggatatggatccgattcgaccttgccgatccaaacccgagccccggtttatcctggttatctccgtagtctgcatcgttgtactaatcggagctttggtttggctttttattaaaaccaaaccgttattccagagaaaaccgaaccggaccgataaagtaaccattttccagcggatcgggttcactgaggaagacatatacccgcaattaacagatgataacataatcgggtcgggcgggtcgggtttggtttacagagtgacactaaagtcaggccaaacgctcgcggtgaagaaactttggggtggaccgggtcaaaaacccgaatccgaatctgttttcagatctgaggtagagattctgggtcgggtcagacatggtaacatagtgaagcttctcatgtgttgcagcggcgaggagtttcggtttttagtgtacgagtatatggaaaacggcagcttgggcgacgttttgcattcggagaaagaacatcgtgccgtttctccgcttgattggacgacacggttttcgatcgcgctgggtgcagctcaaggacttgcttacctacatcatgactctgttccgccgattgttcaccgcgacgtcaaaagcaataatatattgttggaccatgagatgaagccacgtgttgctgatttcggtttagctaaaccgttgagaagagaagtcaataatggtgtctcggatgtttctcctatgtcttgtgttgctggatcctacggctacattgctcaatatggttatacgtcgagagtgaatgagaagagcgatgtgtatagttttggtgtcgtattactcgagttgattacgggtaaaagaccaaatgattcgtcgtttggggagaataaagacattgttaagtttgcaatggagtcagctttgtcttactcttctccatcacctgaagacaaagctatgactcaagattcacctggaaattgtcgagatcttagtaagcttgtggatccaaagatggaactttctaggggggagtatgaagaggtagataaagtttttgagattgcgttgctttgcacgtcttcgtttccgattagtaggccgaccatgaggaaagtagtagagctgctcaaagagaagaagccacttgagtga

SEQ ID NO: 9 - protein, 989 aa, Brassica napus
  source 1..989: note = BnaAHSL2 aa
MTTTITTTSLFLLLVLSCVLQASSNGDAEILSRVKTSRLSDPDGKLNDWVITGDNRSPCNWTGITCDSKNGAVTAIDLSDYGISGGFPYGFCRIRTLVNITLSKNNLNGTIDSAPLTLCSRIHVLILTENSFSGKLPEFSPEFRNLRVLELESNFFSGEIPESYGKLPSLQVLNLNGNSLGGIVPAFLGNLTELTRLELAYVQFEPGPIPSTFGNLTKMTYLRLTNSNIVGEIPDSIGNLVSLVNLDLAQNGLSGEIPESIGKLKSIYQMVLYINQLSGKLPESIGNLTAMRNFDVSQNNLSGDLPETIAALQVVSFHLNDNLFTGELPRGIALNPNLVDFKIFNNSFTGSLPTSFGKFSGLTEFDVSTNRFSGELPPYLCYGKKLEKLIIFSNQLSGEIPETYGECDTLNYIRMADNKLSGEVPVKFWELPLTRLELSNNRLEGSIPPSISKARQLSQLEISGNKLSGAIPARICDLEGLRDVDLSRNRFSGSIPSCINRLKNLERVEMQENMLDGEIPSSVSSCAKLTELNLSDNRLRGEIPPELGELPVLNYLDLSNNQLSGEIPAELLKLKLNLFNVSDNKLSGKIPSGFQQDVFLPSFLGNPGLCAPDMDPIRPCRSKPEPRFILVISVVCIVVLIGALVWLFIKTKPLFQRKPNRTDKVTIFQRIGFTEEDIYPQLTDDNIIGSGGSGLVYRVTLKSGQTLAVKKLWGGPGQKPESESVFRSEVEILGRVRHGNIVKLLMCCSGEEFRFLVYEYMENGSLGDVLHSEKEHRAVSPLDWTTRFSIALGAAQGLAYLHHDSVPPIVHRDVKSNNILLDHEMKPRVADFGLAKPLRREVNNGVSDVSPMSCVAGSYGYIAQYGYTSRVNEKSDVYSFGVVLLELITGKRPNDSSFGENKDIVKFAMESALSYSSPSPEDKAMTQDSPGNCRDLSKLVDPKMELSRGEYEEVDKVFEIALLCTSSFPISRPTMRKVVELLKEKKPLE

SEQ ID NO: 10 - DNA, 3176 bp, Brassica napus
  source 1..3176: mol_type = genomic DNA; note = BnaCHSL2 gene
atgaccaccgttactacttctctcttcttgttacttgttctttcatgtgttctccaagtttcttccaacggagacgcagagatactgagtcgagtcaaaaccagcagactttctgacccagatggaaagttaaatgattgggtcataaccggagataaccggaatccctgtaactggactggaatcacatgcgactccaaaaacggcgccgtcacggcaatagatctctccgactacggagtctccggtgggttcccgtacgggttgtgccgtatacgtaccctcgtcaacatcactctttctaaaaacaatctcaacggtacgatagattcatctcctctctcactatgctctcgaatccacgtcctgatcctcacagaaaacagcttctccgggaacttaccggagttttcgccggagtttcgtaacctacgcgttctcgaactggaatccaactttttctccggcgagattcccgagagctacggaaagcttacatctctgcaagttctgaatctcaacggcaactcgctcgccggaatcgctccggcgtttttgggtaacctgaccgagttgactcgtctcgagctcgcgtacgttcagttcgagcctggtccgatcccgtcggcgttcgggagcttggcgaagctgagtatcctccggctaacgaactcgaacttcgtcggagagattcctgattcgatcgggaatctagtctcgctagtgaatctcgacttagctgagaacggtctctccggagagatgccggagagcatcggaaagctaaaatcgatttatcagctggtgcttttcgagaataacctatcgggaaaattgccggagggtatcggaaacttaacggctatgaggaatttcgacgtttcgcagaatagtctctccggtgagttacctgagacgatcgctgctctccagctcgtttctttccatctcaacgataacttcttcaccggtgaattgccacgtggcattgctttgaatcctaatctcgttgatttcaagatcttcaacaacagtttcacggggagtttacctaggagtttcggtaaattctccggtttaactgagttcgacgtctcgacgaataatttctccggtgagttgccgccgtatctatgctatgggaagaaacttgaaaagctaattagtttcagtaatcagttaagcggcgagatcccggaaacttacggtgagtgcgactcgcttaattatattagaatggcggataacaaactctccggcgaagttccggttaggttttcggagcttcctcttactcgtctcgagctatccaacaaccgattacaaggttcgattcctccgtcgatatccaaagctcgtcagctctctcagctcgaaatctccggaaacaagttctccggtgcgattccggtgagaatctgtgatctcggaggactcagggtcgtcgatctcagccgtaaccgtttctcaggatctatcccgtcttgcattaacagattgaagaatctggagagattggagatgcaggagaacatgctcgacggcgagatcccgagttcggtgagttcatgcgccaagttaaccgagttgaatctctccgataaccgcctccgcggcggaatcccaccggagctcggggaattaccggttttgaactatctggatctctccaacaaccaactcaccggcgaggttccggcggagttgttgaagctgaagcttaatcagttcaacgtctccgataacaaactctccggcaagatcccttctggttttcaacaagatatttttctaccgagttttttgggtaacccggatctctgcgccccggatatggatccgattcgaccttgccgatccaaacccgagccccggtttatcctttttatctccgtcgtctgcatcgttgcactaatcggagctttggtttggctttttatcaaaaccaaaccgttattccagagaaaaccgaaccggaccgataaagtaaccattttccagcggatcgggttcacagaggaagacatatacccgcaattaacagaagataacataatcgggtcgggcgggtcgggtttggtttacagagtgacactaaagtcaggccaaacgctcgcggtgaagaaactttggggtggaccgggtcataaacccgaatccgaatccgttttcagatccgaggtagagattctgggtcgggtcagacatggtaacatagtgaagcttctcatgtgctgcagcggcgaagagtttcggtttctagtgtacgagcatatggaaaacggcagcttgggcgacgttttgcattcagagaaagaacatcgcgccgtttctccgctggattggacgacaaggttttcgatcgcgcttggtgcagctcaaggacttgcttatctacatcatgactctgttccggcgattgttcaccgtgacgtcaaaagcaataatatattgttggaccatgagatgaagccacgtgttgctgatttcggtttggctaaaccgttgaaaagagaagacaataatggtgtctttgatgtttctcctatgtcttgtgttgctggatcctacggctacattgctccaggttcgaattctaaatgctaaaccgaaataacaaatattttttactgttattttttattctgttgttgtgattgcacatgcagaatatggttatacgtcgagagtgaatgagaagagcgatgtgtatagtttcggtgtcgtgttactcgaattgattacgggtaaaaggccaaatgattcttcgtttggggagaataaagacattgttaagtttgcaatggagtcagctttgtcttactcttctccatcacctgaagacaaagctatgaatcaagattcacttggaaattttcgagatcttcgcaaggttgtggatccaaagatggaactttctacgagggaatatgaagaggtagagagagttctcgagattgcattactttgcacgtcttcgtttcctattagtaggccgaccatgaggaaagtagtagagctgctcaaagagaagaagccacttgagtgagacggtgctcgtttcttcatctggttttaatactaagattgttacttaatcataggtgggctactggaaaactaattttaaaaacttaaatagctagttatctttttttttgttaatttaaagatta

SEQ ID NO: 11 - DNA, 2953 bp, Brassica napus
  source 1..2953: mol_type = other DNA; note = BnaCHSL2 cDNA
atgaccaccgttactacttctctcttcttgttacttgttctttcatgtgttctccaagtttcttccaacggagacgcagagatactgagtcgagtcaaaaccagcagactttctgacccagatggaaagttaaatgattgggtcataaccggagataaccggaatccctgtaactggactggaatcacatgcgactccaaaaacggcgccgtcacggcaatagatctctccgactacggagtctccggtgggttcccgtacgggttgtgccgtatacgtaccctcgtcaacatcactctttctaaaaacaatctcaacggtacgatagattcatctcctctctcactatgctctcgaatccacgtcctgatcctcacagaaaacagcttctccgggaacttaccggagttttcgccggagtttcgtaacctacgcgttctcgaactggaatccaactttttctccggcgagattcccgagagctacggaaagcttacatctctgcaagttctgaatctcaacggcaactcgctcgccggaatcgctccggcgtttttgggtaacctgaccgagttgactcgtctcgagctcgcgtacgttcagttcgagcctggtccgatcccgtcggcgttcgggagcttggcgaagctgagtatcctccggctaacgaactcgaacttcgtcggagagattcctgattcgatcgggaatctagtctcgctagtgaatctcgacttagctgagaacggtctctccggagagatgccggagagcatcggaaagctaaaatcgatttatcagctggtgcttttcgagaataacctatcgggaaaattgccggagggtatcggaaacttaacggctatgaggaatttcgacgtttcgcagaatagtctctccggtgagttacctgagacgatcgctgctctccagctcgtttctttccatctcaacgataacttcttcaccggtgaattgccacgtggcattgctttgaatcctaatctcgttgatttcaagatcttcaacaacagtttcacggggagtttacctaggagtttcggtaaattctccggtttaactgagttcgacgtctcgacgaataatttctccggtgagttgccgccgtatctatgctatgggaagaaacttgaaaagctaattagtttcagtaatcagttaagcggcgagatcccggaaacttacggtgagtgcgactcgcttaattatattagaatggcggataacaaactctccggcgaagttccggttaggttttcggagcttcctcttactcgtctcgagctatccaacaaccgattacaaggttcgattcctccgtcgatatccaaagctcgtcagctctctcagctcgaaatctccggaaacaagttctccggtgcgattccggtgagaatctgtgatctcggaggactcagggtcgtcgatctcagccgtaaccgtttctcaggatctatcccgtcttgcattaacagattgaagaatctggagagattggagatgcaggagaacatgctcgacggcgagatcccgagttcggtgagttcatgcgccaagttaaccgagttgaatctctccgataaccgcctccgcggcggaatcccaccggagctcggggaattaccggttttgaactatctggatctctccaacaaccaactcaccggcgaggttccggcggagttgttgaagctgaagcttaatcagttcaacgtctccgataacaaactctccggcaagatcccttctggttttcaacaagatatttttctaccgagttttttgggtaacccggatctctgcgccccggatatggatccgattcgaccttgccgatccaaacccgagccccggtttatcctttttatctccgtcgtctgcatcgttgcactaatcggagctttggtttggctttttatcaaaaccaaaccgttattccagagaaaaccgaaccggaccgataaagtaaccattttccagcggatcgggttcacagaggaagacatatacccgcaattaacagaagataacataatcgggtcgggcgggtcgggtttggtttacagagtgacactaaagtcaggccaaacgctcgcggtgaagaaactttggggtggaccgggtcataaacccgaatccgaatccgttttcagatccgaggtagagattctgggtcgggtcagacatggtaacatagtgaagcttctcatgtgctgcagcggcgaagagtttcggtttctagtgtacgagcatatggaaaacggcagcttgggcgacgttttgcattcagagaaagaacatcgcgccgtttctccgctggattggacgacaaggttttcgatcgcgcttggtgcagctcaaggacttgcttatctacatcatgactctgttccggcgattgttcaccgtgacgtcaaaagcaataatatattgttggaccatgagatgaagccacgtgttgctgatttcggtttggctaaaccgttgaaaagagaagacaataatggtgtctttgatgtttctcctatgtcttgtgttgctggatcctacggctacattgctcgttatacgtcgagagtgaatgagaagagcgatgtgtatagtttcggtgtcgtgttactcgaattgattacgggtaaaaggccaaatgattcttcgtttggggagaataaagacattgttaagtttgcaatggagtcagctttgtcttactcttctccatcacctgaagacaaagctatgaatcaagattcacttggaaattttcgagatcttcgcaaggttgtggatccaaagatggaactttctacgagggaatatgaagaggtagagagagttctcgagattgcattactttgcacgtcttcgtttcctattagtaggccgaccatgaggaaagtagtagagctgctcaaagagaagaagccacttg

SEQ ID NO: 12 - protein, 985 aa, Brassica napus
  source 1..985: note = BnaCHSL2 aa
MTTVTTSLFLLLVLSCVLQVSSNGDAEILSRVKTSRLSDPDGKLNDWVITGDNRNPCNWTGITCDSKNGAVTAIDLSDYGVSGGFPYGLCRIRTLVNITLSKNNLNGTIDSSPLSLCSRIHVLILTENSFSGNLPEFSPEFRNLRVLELESNFFSGEIPESYGKLTSLQVLNLNGNSLAGIAPAFLGNLTELTRLELAYVQFEPGPIPSAFGSLAKLSILRLTNSNFVGEIPDSIGNLVSLVNLDLAENGLSGEMPESIGKLKSIYQLVLFENNLSGKLPEGIGNLTAMRNFDVSQNSLSGELPETIAALQLVSFHLNDNFFTGELPRGIALNPNLVDFKIFNNSFTGSLPRSFGKFSGLTEFDVSTNNFSGELPPYLCYGKKLEKLISFSNQLSGEIPETYGECDSLNYIRMADNKLSGEVPVRFSELPLTRLELSNNRLQGSIPPSISKARQLSQLEISGNKFSGAIPVRICDLGGLRVVDLSRNRFSGSIPSCINRLKNLERLEMQENMLDGEIPSSVSSCAKLTELNLSDNRLRGGIPPELGELPVLNYLDLSNNQLTGEVPAELLKLKLNQFNVSDNKLSGKIPSGFQQDIFLPSFLGNPDLCAPDMDPIRPCRSKPEPRFILFISVVCIVALIGALVWLFIKTKPLFQRKPNRTDKVTIFQRIGFTEEDIYPQLTEDNIIGSGGSGLVYRVTLKSGQTLAVKKLWGGPGHKPESESVFRSEVEILGRVRHGNIVKLLMCCSGEEFRFLVYEHMENGSLGDVLHSEKEHRAVSPLDWTTRFSIALGAAQGLAYLHHDSVPAIVHRDVKSNNILLDHEMKPRVADFGLAKPLKREDNNGVFDVSPMSCVAGSYGYIARYTSRVNEKSDVYSFGVVLLELITGKRPNDSSFGENKDIVKFAMESALSYSSPSPEDKAMNQDSLGNFRDLRKVVDPKMELSTREYEEVERVLEIALLCTSSFPISRPTMRKVVELLKEKKPLE

SEQ ID NO: 13 - DNA, 3351 bp, Brassica rapa
  source 1..3351: mol_type = genomic DNA; note = BraAHAE gene
atgctttattgtcttattctcctcctcctcctatgcctctcttcaacgtatctatgcctctcactaaaccacgatgccaccatcctccggcaagccaaactgggcttatctgacccggcccagtcactatcttcctggtccgaaaacgacgtcactccgtgccactggcgcggcatcacctgcgacgccacgtcagctgtcttctccgttaatctctccagcttcatgctcgtcggtcctttcccctccgtcctctgccgtcttccttcgctgtctttcctctctctctacaacaactccatcaacggttcgctctccggcgatgagtttacatcgtgtcggaatctcagccaactcgatttgtcggagaatctcttggttggttccatcccggagtcacttcctttcacgctgccgaacctccgacacttcgaagtttccgggaacaacttatcagatacgattccagcgagcttcggagagttccgcaagctcgagaagttaaacctcgccggaaacctccctccggtaccattcccgcctctctcggaaacgtatccaccctgagagaactcaaactcgcttacaatctattttcaccgggtagaatcccgagtcaactcggtaatctaacggagcttcgtgttctctggctcgccggctgcaacctcgctggtccggtgcctcaagctctgtcccggttgactcacttggtcagcttggatttgacgatgaatcaactcaccgggtctgtcccgagttggatcacggagctgaaaagcatcgagcaaatcgagatattcaacaactcgttctcaggcgtgttaccggaggcgatgggtaacatgacgatgctaaagagtttcgacgcgtcgatgaacaagctgacggggaagattcccgacggcttgactcggttaaacctcgagtcgctcaacctcttcgagaacatgctcgaagggcccttgccggagagcattactcgctccaaaacgttaacggaactaaaactgttcaacaacaaactcacaggggagataccgagtcaactcggggcgagctccccattgcagttcgtggacttttcgtataatcagttttccggcgagatacctgcgaatatatgcggaggagggaagctagagtttctcatgcttatagggaactctttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcgatctcagctctaatcagctctccggcgagattccatgtggaattcgtggttggaagaatctgaacgagctcaatttagctaataaccatctctccggcgaagtccctagagagctcggagacttgccggttcttaactacctcgatctctcaaacaatcaattttccggtgagattccaccggagctccagaatctgaagctcaacgttataaacctatcgtataatcatctctccgggagagtgcctcctctctacgctaacaaaatctacgattctagctttgtcggaaaccatgacttgtgcgtcgatgatcacgacagtcgttgtcggaagaagagcacacggtctcaaaacatcggttatgtctggattctcctatcgattttcatactcgcctgtatggttttcgtcgttggggttgttatgtttatcgccaactgcaagaagatgagagcctccaagagcgctagattctccgcatctaagtggagatccttccacaagcttcatttcagcgagcatgagatcgttgattgccttgacgaacggaacgtgatcggatctggctcctccggtaaagtctatagagtggagcttagtggcggagaagtagtagtagctgtgaagaaactaaacaaaacggctaaaggaggggaagacgattcgttgaacagagacgttttcgctgcagaggtggaaacgcttggaacgattaggcataagagtattgtgcgcttgtggtgctgctgcagctccggggactgcaagttgttggtatacgagtacatgcctaatgggagcttagctgatgtgttacacagccactgcaaaggcggggtgttgttgggctggcctgaacggttaaggatcgctttggacgcagctgagggtttgtcatacttacaccacgattgtgttcctccgatcgtgcaccgtgacgtgaagtctagtaacatattgctagatgcagattatggagccaaagtagctgactttggcattgcaacaatcggtcaaatgagcggtactaaatcgccagaagctatgtctggaatcgctggttcttgcggttacattgcaccaggtatgataacataacaataaaatgataaaaccatacatttacataatgtttcaaatgatattggtgcgttaatagtttttttctttctaatgtttctgcagaatacgtatatacactcagggtgaatgaaaagagtgatatctacagttttggtatcgtgcttttggagttggtaacggggaagcaaccaacagatctagaacttggagataaagatatggtgaagtgggtgtgcactacacttgaccaaagcggtttagaatcggtgatcgatcccaaacttgatctgttcaaagaagagattagcaaactcattcacattggtcttctctgtacgagtcctctccctctaaaccgaccttccatgagaaaagtcgtgatcatgcttcaagaagtctctagtgctgtttctagtagcggtccaaatgcgtccaaacgctctaagagcagtgggaaactctcgccttactatgtggaagacgtaaacagcgtttgagcattcaaatatgggccggtatgagttgcgctgggccagaaatagttatttggtttgtattaggttaggtgaattgtaagaaatcagaaacagaaaaatggagctttctgattaggcaatagcttttaatgctatgactagattttgacccgcgcaggcgcgcgggtgtatattttgaaaaatatgttgatatttgtttttcatgtaattattaagatttggaaaaatgaatccgaggaacataaccgataccgatc

SEQ ID NO: 14 - DNA, 2951 bp, Brassica rapa
  source 1..2951: mol_type = other DNA; note = BraAHAE cDNA
atgctttattgtcttattctcctcctcctcctatgcctctcttcaacgtatctatgcctctcactaaaccacgatgccaccatcctccggcaagccaaactgggcttatctgacccggcccagtcactatcttcctggtccgaaaacgacgtcactccgtgccactggcgcggcatcacctgcgacgccacgtcagctgtcttctccgttaatctctccagcttcatgctcgtcggtcctttcccctccgtcctctgccgtcttccttcgctgtctttcctctctctctacaacaactccatcaacggttcgctctccggcgatgagtttacatcgtgtcggaatctcagccaactcgatttgtcggagaatctcttggttggttccatcccggagtcacttcctttcacgctgccgaacctccgacacttcgaagtttccgggaacaacttatcagatacgattccagcgagcttcggagagttccgcaagctcgagaagttaaacctcgccggaaacctcctctccggtaccattcccgcctctctcggaaacgtatccaccctgagagaactcaaactcgcttacaatctattttcaccgggtagaatcccgagtcaactcggtaatctaacggagcttcgtgttctctggctcgccggctgcaacctcgctggtccggtgcctcaagctctgtcccggttgactcacttggtcagcttggatttgacgatgaatcaactcaccgggtctgtcccgagttggatcacggagctgaaaagcatcgagcaaatcgagatattcaacaactcgttctcaggcgtgttaccggaggcgatgggtaacatgacgatgctaaagagtttcgacgcgtcgatgaacaagctgacggggaagattcccgacggcttgactcggttaaacctcgagtcgctcaacctcttcgagaacatgctcgaagggcccttgccggagagcattactcgctccaaaacgttaacggaactaaaactgttcaacaacaaactcacaggggagataccgagtcaactcggggcgagctccccattgcagttcgtggacttttcgtataatcagttttccggcgagatacctgcgaatatatgcggaggagggaagctagagtttctcatgcttatagggaactctttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcatctcagctctaatcagctctccggcgagattccatgtggaattcgtggttggaagaatctgaacgagctcaatttagctaataaccatctctccggcgaagtccctagagagctcggagacttgccggttcttaactacctcgatctctcaaacaatcaattttccggtgagattccaccggagctccagaatctgaagctcaacgttataaacctatcgtataatcatctctccgggagagtgcctcctctctacgctaacaaaatctacgattctagctttgtcggaaaccatgacttgtgcgtcgatgatcacgacagtcgttgtcggaagaagagcacacggtctcaaaacatcggttatgtctggattctcctatcgattttcatactcgcctgtatggttttcgtcgttggggttgttatgtttatcgccaactgcaagaagatgagagcctccaagagcgctagattctccgcatctaagtggagatccttccacaagcttcatttcagcgagcatgagatcgttgattgccttgacgaacggaacgtgatcggatctggctcctccggtaaagtctatagagtggagcttagtggcggagaagtagtagtagctgtgaagaaactaaacaaaacggctaaaggaggggaagacgattcgttgaacagagacgttttcgctgcagaggtggaaacgcttggaacgattaggcataagagtattgtgcgcttgtggtgctgctgcagctccggggactgcaagttgttggtatacgagtacatgcctaatgggagcttagctgatgtgttacacagccactgcaaaggcggggtgttgttgggctggcctgaacggttaaggatcgctttggacgcagctgagggtttgtcatacttacaccacgattgtgttcctccgatcgtgcaccgtgacgtgaagtctagtaacatattgctagatgcagattatggagccaaagtagctgactttggcattgcaacaatcggtcaaatgagcggtactaaatcgccagaagctatgtctggaatcgctggttcttgcggttacattgcaccagaatacgtatatacactcagggtgaatgaaaagagtgatatctacagttttggtatcgtgcttttggagttggtaacggggaagcaaccaacagatctagaacttggagataaagatatggtgaagtgggtgtgcactacacttgaccaaagcggtttagaatcggtgatcgatcccaaacttgatctgttcaaagaagagattagcaaactcattcacattggtcttctctgtacgagtcctctccctctaaaccgaccttccatgagaaaagtcgtgatcatgcttcaagaagtctctagtgctgtttctagtagcggtccaaatgcgtccaaacgctctaagagcagtggg

SEQ ID NO: 15 - protein, 997 aa, Brassica rapa
  source 1..997: note = BraAHAE aa
MLYCLILLLLLCLSSTYLCLSLNHDATILRQAKLGLSDPAQSLSSWSENDVTPCHWRGITCDATSAVFSVNLSSFMLVGPFPSVLCRLPSLSFLSLYNNSINGSLSGDEFTSCRNLSQLDLSENLLVGSIPESLPFTLPNLRHFEVSGNNLSDTIPASFGEFRKLEKLNLAGNLLSGTIPASLGNVSTLRELKLAYNLFSPGRIPSQLGNLTELRVLWLAGCNLAGPVPQALSRLTHLVSLDLTMNQLTGSVPSWITELKSIEQIEIFNNSFSGVLPEAMGNMTMLKSFDASMNKLTGKIPDGLTRLNLESLNLFENMLEGPLPESITRSKTLTELKLFNNKLTGEIPSQLGASSPLQFVDFSYNQFSGEIPANICGGGKLEFLMLIGNSFSGEIPINLGKCRSLTRVRLNNNKLSGHVPEEFWGLPRMSLLELSENSFTGRISESIAGAKNLSNLRISKNQFSGSIPGEIGSLNGLIEITGDENSFSGEVPSTLVKLKQLSRLDLSSNQLSGEIPCGIRGWKNLNELNLANNHLSGEVPRELGDLPVLNYLDLSNNQFSGEIPPELQNLKLNVINLSYNHLSGRVPPLYANKIYDSSFVGNHDLCVDDHDSRCRKKSTRSQNIGYVWILLSIFILACMVFVVGVVMFIANCKKMRASKSARFSASKWRSFHKLHFSEHEIVDCLDERNVIGSGSSGKVYRVELSGGEVVVAVKKLNKTAKGGEDDSLNRDVFAAEVETLGTIRHKSIVRLWCCCSSGDCKLLVYEYMPNGSLADVLHSHCKGGVLLGWPERLRIALDAAEGLSYLHHDCVPPIVHRDVKSSNILLDADYGAKVADFGIATIGQMSGTKSPEAMSGIAGSCGYIAPEYVYTLRVNEKSDIYSFGIVLLELVTGKQPTDLELGDKDMVKWVCTTLDQSGLESVIDPKLDLFKEEISKLIHIGLLCTSPLPLNRPSMRKVVIMLQEVSSAVSSSGPNASKRSKSSGKLSPYYVEDVNSV

SEQ ID NO: 16 - DNA, 2970 bp, Brassica rapa
  source 1..2970: mol_type = genomic DNA; note = BraAHSL2 gene
atgaccaccaccattactactacttctctctttttgttacttgttctctcatgtgttctccaagcttcttccaacggagacgctgagatactgagcagagtcaaaaccagcagactttctgacccggatggaaagttaaatgattgggtcataaccggagataaccggagtccctgtaactggactggaatcacatgcgactccaaaaacggcgccgtcacggcaatagatctctccgactacggaatctccggtgggttcccgtacgggttctgccgtatacgtaccctcgtcaacatcactctctctaaaaacaatctcaacggtacgatagattcagctcctctcacactatgctctcgaatccacgtcctgatcctcacagaaaacagcttctccgggaagctaccggagttttcgccggagtttcgcaacctacgcgttctcgaactggagtccaactttttctccggcgagatccccgaaagctacggaaagcttccatctctgcaagttctgaatctcaacggcaactcgctcggcggaatcgttccggcgtttttgggtaacctgaccgagttgactcgcctcgagctcgcgtacgttcagttcgagcctggtccgattccgtcgacgtttgggaacttgacgaagatgacttatctccggctaactaactcgaacatcgtcggagagattcctgattcgatcgggaatctggtttccctggtgaatctcgatttagctcagaacggtctctccggagagataccggagagcatcggaaagctaaaatcgatttatcaaatggtgctttacatcaatcagctatcgggaaaattgccggagagtatcggaaacttaacggctatgaggaatttcgacgtttcgcagaataatctctccggtgatttgcctgagacgatcgccgctctccaggtcgtttctttccatctcaatgataatttattcaccggtgaattgccacgtggcattgctttgaatcctaatctcgttgatttcaagatctttaacaacagtttcaccgggagtttacctacgagtttcggtaaattctccggtttaactgagttcgacgtctcgactaacagattctccggtgagttgcctccgtatctatgctacgggaagaaacttgaaaagctaatcattttcagcaatcagttaagcggcgagattccggaaacgtacggtgagtgcgacacgcttaattatattagaatggcggataacaaactctccggcgaagttccggttaagttttgggagcttcctctcactcgtctcgagctatctaacaaccgattagaaggttcgattcctccgtcgatatccaaagctcgtcagctctctcagctcgagatctccggaaacaagttgtccggtgcgattccggcgagaatctgtgatctcgaaggactcagggacgtcgatctcagccgtaaccgtttctcaggatctattccctcttgcattaaccgattgaagaatctggagagagtggagatgcaggagaacatgctcgacggcgagatcccgagttcggtgagttcatgcgccaagttaacggagttgaatctctccgataaccgtctccgcggcgaaattccaccggagctcggtgagttaccggttttgaactatctggatctctccaacaaccaactctccggtgagatcccggcggagttgttgaagctgaagctgaatctgttcaacgtctccgataacaaactctccgggaagataccttctggttttcaacaagatgtttttctaccgagttttttgggtaacccgggactctgtgctccggatatggatccgattcgaccttgccgatccaaacccgagccccggtttatcctggttatctccgtagtctgcatcgttgtactaatcggagctttggtttggctttttattaaaaccaaaccgttattccagagaaaaccgaaccggaccgataaagtaaccattttccagcggatcgggttcactgaggaagacatatacccgcaattaacagatgataacataatcgggtcgggcgggtcgggtttggtttacagagtgacactaaagtcaggccaaacgctcgcggtgaagaaactttggggtggaccgggtcaaaaacccgaatccgaatctgttttcagatctgaggtagagattctgggtcgggtcagacatggtaacatagtgaagcttctcatgtgttgcagcggcgaggagtttcggtttttagtgtacgagtatatggaaaacggcagcttgggcgacgttttgcattcggagaaagaacatcgtgccgtttctccgcttgattggacgacacggttttcgatcgcgctgggtgcagctcaaggacttgcttacctacatcatgactctgttccgccgattgttcaccgcgacgtcaaaagcaataatatattgttggaccatgagatgaagccacgtgttgctgatttcggtttagctaaaccgttgagaagagaagtcaataatggtgtctcggatgtttctcctatgtcttgtgttgctggatcctacggctacattgctccagaatatggttatacgtcgagagtgaatgagaagagcgatgtgtatagttttggtgtcgtattactcgagttgattacgggtaaaagaccaaatgattcgtcgtttggggagaataaagacattgttaagtttgcaatggagtcagctttgtcttactcttctccatcacctgaagacaaagctatgactcaagattcacctggaaattgtcgagatcttagtaagcttgtggatccaaagatggaactttctaggggggagtatgaagaggtagataaagtttttgagattgcgttgctttgcacgtcttcgtttccgattagtaggccgaccatgaggaaagtagtagagctgctcaaagagaagaagccacttgag

SEQ ID NO: 17 - DNA, 2968 bp, Brassica rapa
  source 1..2968: mol_type = other DNA; note = BraAHSL2 cDNA
atgaccaccaccattactacttctctcttcttgttacttgttctttcatgcgttctccaagcttcttccaacggagacgctgagatactgagcagagtcaaaaccagcagactttctgacccggaaggaaagttaaatgattgggtcataaccggagataaccggaatccctgtaactggactggaatcacatgcgactccaaaaacggccgtcacggcaatagatctctccgactacggaatctccggtgggttcccgtacgggttctgccgtatacgtacgctcatcaacatcactctttctaaaaacaatctcaacggtacgatagattcatctcctctctcactatgctctcgaatccacgtcctgatcctcacagaaaacagcttctccgggaacttaccggagttttcgccggagtttcgtaacctacgcgttctcgaactggagtccaactttttctccggcgagatccccgaaagctacggaaagtttgcatctctgcaagttctgaatctcaacggcaactcgctcggcggaatcgttccggcattcttgggtaacctgaccgagttgactcgcctcgagctcgcgtacgttcagttcgagcctggtccgattccgtcgacgtttgggaacttgacgaagatgacttatctccggctaactaactcgaacatcgtcggagagattcctgattcgatcgggaatctggtttccctggtgaatctcgatttagctcagaacggtctctccggagagataccggagagcatcggaaagctaaaatcgatttatcaaatggtgctttacatcaatcagctatcgggaaaattgccggagagtatcggaaacttaacggctatgaggaatttcgacgtttcgcagaataatctctccggtgatttgcctgagacgatcgccgctctccaggtcgtttctttccatctcaatgataatttattcaccggtgaattgccacgtggcattgctttgaatcctaatctcgttgatttcaagatctttaacaacagtttcaccgggagtttacctacgagtttcggtaaattctccggtttaactgagttcgacgtctcgactaacagattctccggtgagttgcctccgtatctatgctacgggaagaaacttgaaaagctaatcattttcagcaatcagttaagcggcgagattccggaaacgtacggtgagtgcgacacgcttaattatattagaatggcggataacaaactctccggcgaagttccggttaagttttgggagcttcctctcactcgtctcgagctatctaacaaccgattagaaggttcgattcctccgtcgatatccaaagctcgtcagctctctcagctcgagatctccggaaacaagttgtccggtgcgattccggcgagaatctgtgatctcgaaggactcagggacgtcgatctcagccgtaaccgtttctcaggatctattccctcttgcattaaccgattgaagaatctggagagagtggagatgcaggagaacatgctcgacggcgagatcccgagttcggtgagttcatgcgccaagttaacggagttgaatctctccgataaccgtctccgcggcgaaattccaccggagctcggtgagttaccggttttgaactatctggatctctccaacaaccaactctccggtgagatcccggcggagttgttgaagctgaagctgaatctgttcaacgtctccgataacaaactctccgggaagataccttctggttttcaacaagatgtttttctaccgagttttttgggtaacccgggactctgtgctccggatatggatccgattcgaccttgccgatccaaacccgagccccggtttatcctggttatctccgtagtctgcatcgttgtactaatcggagctttggtttggctttttattaaaaccaaaccgttattccagagaaaaccgaaccggaccgataaagtaaccattttccagcggatcgggttcactgaggaagacatatacccgcaattaacagatgataacataatcgggtcgggcgggtcgggtttggtttacagagtgacactaaagtcaggccaaacgctcgcggtgaagaaactttggggtggaccgggtcaaaaacccgaatccgaatctgttttcagatctgaggtagagattctgggtcgggtcagacatggtaacatagtgaagcttctcatgtgttgcagcggcgaggagtttcggtttttagtgtacgagtatatggaaaacggcagcttgggcgacgttttgcattcggagaaagaacatcgtgccgtttctccgcttgattggacgacacggttttcgatcgcgctgggtgcagctcaaggacttgcttacctacatcatgactctgttccgccgatttttcaccgcgacgtcaaaagcaataatatattgttggaccatgagatgaagccacgtgttgctgatttcggtttagctaaaccgttgagaagagaagtcaataatggtgtctcggatgtttctcctatgtcttgtgttgctggatcctacggctacattgctccagaatatggttatacgtcgagagtgaatgagaagagcgatgtgtatagttttggtgtcgtattactcgagttgattacgggtaaaagaccaaatgattcgtcgtttggggagaataaagacattgttaagtttgcaatggagtcagctttgtcttactcttctccatcacctgaagacaaagctatgactcaagattcacctggaaattgtcgagatcttagtaagcttgtggatccaaagatggaactttctaggggggagtatgaagaggtagataaagtttttgagattgcgttgctttgcacgtcttcgtttccgattagtaggccgaccatgaggaaagtagtagagctgctcaaagagaagaagccacttgagtga

SEQ ID NO: 18 - protein, 987 aa, Brassica rapa
  source 1..987: note = BraAHSL2 aa
MTTTITTFLFLLVLSCVLQASSNGDAEILSRVKTSRLSDPDGKLNDWVITGDNRSPCNWTGITCDSRNAVTAIDLSEYGISGGFPYGFCRIRTLVNITLSKNNLNGTIDSAPLSLCSRIHVLILTENSFSGNLPEFSPEFRNLRVLELESNFFSGEIPESYGKLTSLQALNLNGNSLGGIVPAFLGNLTELTRLELAYVQFEPGPIPSTFGNLTKMTYLRLTNSNIVGEIPDSIGNLVSLVNLDLAQNGLSGEIPESIGKLKSIYQMVLYINQLSGKLPESIGNLTAMRNFDVSQNNLSGELPETIAALQVVSFHLNDNLFTGELPRGIALNPNLVDFKIFNNSFTGSLPTSFGKFSGLTEFDVSTNRFSGELPPYLCYGKKLEKLIIFSNQLSGEIPETYGECDTLNYIRMADNKLSGEVPVKFWELPLTRLELSNNQLEGSIPPSISKARRLSQLEISGNKLSGAIPARICDLGGLRDVDLSRNRFSGSIPSCINRLKNLERVEMQENMLDGEIPSSVSSCAKLTELNLSDNRLRGEIPPELGELPVLNYLDLSNNQLSGEIPAELLKLKLNLFNVSDNKLSGKIPSGFQQDVFLPSFLGNPGLCAPDMDPIRPCRSKPEPRFILVISVVCIVALIGALVWLFIKTKPLFQRKPNRTDKVTIFQRIGFTEEDIYPQLTEDNIIGSGGSGLVYRVTLKSGQTLAVKKLWGGPGQKPESESVFRSEVEILGRVRHGNIVKLLMCCSGEEFRFLVYEYMENGSLGDVLHSEKEHRAVSPLDWTTRFSIALGAAQGLAYLHHDSVPAIVHRDVKSNNILLDHEMKPRVADFGLAKPLRREVNNGVSDVSPMSCVAGSYGYIAPEYGYTSRVNEKSDVYSFGVVLLELITSKRPNDSSFGENKDIVKFAMESALSYSSPSPEDKAMNQDSPGNCRDLSKLVDPKMELSRGEYGEVEKVFEIALLCTSSFPISRPTMRKVVELLKEKKPLE

SEQ ID NO: 19 - DNA, 3077 bp, Brassica juncea
  source 1..3077: mol_type = genomic DNA; note = BjuAHAE gene
atgctttattatcttattctcctcctcctatgcctctcttctacgtacctatgcctctcaccaaaccacgatgccaccatcctccggcaagccaaactgggcttatccgacccggcccagtcactatcttcctggtccgaaaacgacgtcactccgtgccactggcgcggcatcacctgcgacgccacgtcagctgtcttctccgttaatctctccagcttcatgctcctcggtcctttcccctccgtcctctgccgtcttccttcgctgtctttcctctctctctacaacaactccatcaacggttccctctccggcgatgagtttacatcgtgtcggaatctcagccaactcgatttgtcggagaatctcttggttggttccatcccggagtcacttcctttcacgctgccgaacctccgacacttcgaagtttccgggaacaacttatcagatacgattccagcgagcttcggagagttccgcaagctcgagaagttaaacctcgccggaaacctcctctccggtaccattcccgcctctctcggaaacctatccactctgagagaactcaaactcgcttacaatctgttttcaccgggtcgaatcccgagtcaactcggtaatctaacggagcttcgtgttctctggctcgctggctgcaacctcgctggtccggtgcctcaagctctgtcccggttgactcacttggtcagcttggatttgacgatgaatcaactcaccgggtctgtcctgagttggatcacggagctgaaaagcatcgagcaaatcgagatattcaacaactcgttctcaggcgagttaccggaggcgatgggtaacatgacgatgctaaagaatttcgacgcgtcgacgaacaagctgacggggaagatccccgagggcttggctcggttaaacctcgagtctctcaacctcttcgagaacatgctcgaagggcccttgccggagagcataactcactccaaaacgttaacggaactaaaactgttcaacaacaaactcaccggggagataccgagtcaactcggcgcgagctccccgttgcagttcgtggacttttcgtataatcagttttccggcgagataccggcgaatatatgcggaggagggaagctagagtttctcatgcttatagggaactctttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcgatctcagctctaatcagctctccggcgagattccacgtggaattcgtggttggaagaatctgaacgagctcaatttagctaataaccatctctccggcgaagtctctagagagctcggagacttgccggttcttaactacctcgatctctcaaacaatcaattttccggtgagattccaccggagctccagaatctgaagtcaacgttataaacctatcgtataatcatctctccgggagagtgcctcctctctacgctaacaaaatctacgattctagctttgtcggaaaccatgacttgtgcgtcgatgatcacgacagtctttgtcggaagaagagcacacggtctcaaaacatcggttatgtctggattctcctatcgattttcatactcacctgtatggtttttgttgttggggttgttatgtttatcgccaactgcaagaagatgagagcatccaagagcgctagattctccgcgtccaagtggagatccttccacaagcttcatttcagcgagcatgagatcgttgattgccttgacgaacggaacgtgatcggatctggttcctccggtaaagtctatagagtggagcttagtggcggagaagtagtagtagctgtgaagaaactaaacaaaacggctaaaggaggtgaagacgattcgttgaacagagacgttttcgctgtggaggttgaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttcctccgatcgtgcaccgtgacgtgaagtctagtaacatattgctagatgcagattatggagccaaagtagctgactttggcattgctaaaatcggtcaaatgagcggtgctaaatcgccagaagctatgtctggaatcgccggttcttgcggttacattgcaccaggtataaccaaatcaaatgataaaaagataacttatatttcaaatgttatttgtgctttatagctcttttttatgctgttttgtagaatacgtatatacacttagggtgaatgaaaagagtgatatctacagttttggtatcgtgcttttggagttggtaacggggaagcaaccaacagatctagaacttggagataaagatatggtgaagtgggtgtgctctacccttgaccaatgcggtttagaatcggtgatcgatcccaaacttgatctggggttcaaagaagagattagcaaagtcattcacattggtcttctttgtacgagtcctctccctctaaaccgaccttccatgagaaaagttgtgatcatgcttcaagaagtctctggtgctgtttctagtagcagaccaaatgcgtccaaacgctctaagagcagtgggaaactctcgccttactatgtggaagacgtaaacagcgtttg

SEQ ID NO: 20 - DNA, 2994 bp, Brassica juncea
  source 1..2994: mol_type = other DNA; note = BjuAHAE cDNA
atgctttattatcttattctcctcctcctatgcctctcttctacgtacctatgcctctcaccaaaccacgatgccaccatcctccggcaagccaaactgggcttatccgacccggcccagtcactatcttcctggtccgaaaacgacgtcactccgtgccactggcgcggcatcacctgcgacgccacgtcagctgtcttctccgttaatctctccagcttcatgctcctcggtcctttcccctccgtcctctgccgtcttccttcgctgtctttcctctctctctacaacaactccatcaacggttccctctccggcgatgagtttacatcgtgtcggaatctcagccaactcgatttgtcggagaatctcttggttggttccatcccggagtcacttcctttcacgctgccgaacctccgacacttcgaagtttccgggaacaacttatcagatacgattccagcgagcttcggagagttccgcaagctcgagaagttaaacctcgccggaaacctcctctccggtaccattcccgcctctctcggaaacctatccactctgagagaactcaaactcgcttacaatctgttttcaccgggtcgaatcccgagtcaactcggtaatctaacggagcttcgtgttctctggctcgctggctgcaacctcgctggtccggtgcctcaagctctgtcccggttgactcacttggtcagcttggatttgacgatgaatcaactcaccgggtctgtcctgagttggatcacggagctgaaaagcatcgagcaaatcgagatattcaacaactcgttctcaggcgagttaccggaggcgatgggtaacatgacgatgctaaagaatttcgacgcgtcgacgaacaagctgacggggaagatccccgagggcttggctcggttaaacctcgagtctctcaacctcttcgagaacatgctcgaagggcccttgccggagagcataactcactccaaaacgttaacggaactaaaactgttcaacaacaaactcaccggggagataccgagtcaactcggcgcgagctccccgttgcagttcgtggacttttcgtataatcagttttccggcgagataccggcgaatatatgcggaggagggaagctagagtttctcatgcttatagggaactctttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcgatctcagctctaatcagctctccggcgagattccacgtggaattcgtggttggaagaatctgaacgagctcaatttagctaataaccatctctccggcgaagtctctagagagctcggagacttgccggttcttaactacctcgatctctcaaacaatcaattttccggtgagattccaccggagctccagaatctgaagctcaacgttataaacctatcgtataatcatctctccgggagagtgcctcctctctacgctaacaaaatctacgattctagctttgtcggaaaccatgacttgtgcgtcgatgatcacgacagtctttgtcggaagaagagcacacggtctcaaaacatcggttatgtctggattctcctatcgattttcatactcacctgtatggtttttgttgttggggttgttatgtttatcgccaactgcaagaagatgagagcatccaagagcgctagattctccgcgtccaagtggagatccttccacaagcttcatttcagcgagcatgagatcgttgattgccttgacgaacggaacgtgatcggatctggttcctccggtaaagtctatagagtggagcttagtggcggagaagtagtagtagctgtgaagaaactaaacaaaacggctaaaggaggtgaagacgattcgttgaacagagacgttttcgctgtggaggttgaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttcctccgatcgtgcaccgtgacgtgaagtctagtaacatattgctagatgcagattatggagccaaagtagctgactttggcattgctaaaatcggtcaaatgagcggtgctaaatcgccagaagctatgtctggaatcgccggttcttgcggttacattgcaccagaatacgtatatacacttagggtgaatgaaaagagtgatatctacagttttggtatcgtgcttttggagttggtaacggggaagcaaccaacagatctagaacttggagataaagatatggtgaagtgggtgtgctctacccttgaccaatgcggtttagaatcggtgatcgatcccaaacttgatctggggttcaaagaagagattagcaaagtcattcacattggtcttctttgtacgagtcctctccctctaaaccgaccttccatgagaaaagttgtgatcatgcttcaagaagtctctggtgctgtttctagtagcagaccaaatgcgtccaaacgctctaagagcagtgggaaactctcgccttactatgtggaagacgtaaacagcgtttga

SEQ ID NO: 21 - protein, 997 aa, Brassica juncea
  source 1..997: note = BjuAHAE aa
MLYYLILLLLCLSSTYLCLSPNHDATILRQAKLGLSDPAQSLSSWSENDVTPCHWRGITCDATSAVFSVNLSSFMLLGPFPSVLCRLPSLSFLSLYNNSINGSLSGDEFTSCRNLSQLDLSENLLVGSIPESLPFTLPNLRHFEVSGNNLSDTIPASFGEFRKLEKLNLAGNLLSGTIPASLGNLSTLRELKLAYNLFSPGRIPSQLGNLTELRVLWLAGCNLAGPVPQALSRLTHLVSLDLTMNQLTGSVLSWITELKSIEQIEIFNNSFSGELPEAMGNMTMLKNFDASTNKLTGKIPEGLARLNLESLNLFENMLEGPLPESITHSKTLTELKLFNNKLTGEIPSQLGASSPLQFVDFSYNQFSGEIPANICGGGKLEFLMLIGNSFSGEIPINLGKCRSLTRVRLNNNKLSGHVPEEFWGLPRMSLLELSENSFTGRISESIAGAKNLSNLRISKNQFSGSIPGEIGSLNGLIEITGDENSFSGEVPSTLVKLKQLSRLDLSSNQLSGEIPRGIRGWKNLNELNLANNHLSGEVSRELGDLPVLNYLDLSNNQFSGEIPPELQNLKLNVINLSYNHLSGRVPPLYANKIYDSSFVGNHDLCVDDHDSLCRKKSTRSQNIGYVWILLSIFILTCMVFVVGVVMFIANCKKMRASKSARFSASKWRSFHKLHFSEHEIVDCLDERNVIGSGSSGKVYRVELSGGEVVVAVKKLNKTAKGGEDDSLNRDVFAVEVETLGTIRHKSIVRLWCCCSSGDCKLLVYEYMPNGSLADVLHSHCKGGVLLGWPERLRIGLDAAEGLSYLHHDCVPPIVHRDVKSSNILLDADYGAKVADFGIAKIGQMSGAKSPEAMSGIAGSCGYIAPEYVYTLRVNEKSDIYSFGIVLLELVTGKQPTDLELGDKDMVKWVCSTLDQCGLESVIDPKLDLGFKEEISKVIHIGLLCTSPLPLNRPSMRKVVIMLQEVSGAVSSSRPNASKRSKSSGKLSPYYVEDVNSV

SEQ ID NO: 22 - DNA, 3079 bp, Brassica juncea
  source 1..3079: mol_type = other DNA; note = BjuBHAE gene
atgctttattgtcttattctcctcctcctcctatgcctctcttccacgtatctatgtctctctctaaaccacgatgccaccattctccggcaagctaaactgggcttatccgacccggcccagtcactatcttcgtggtccgaaaacgacgtcacaccgtgccactggcgtggcatcacctgcgacgccacgtcagctgtcttctccgtcgatctctccagcttcacgctcctcggtcctttcccctccgtcctctgccgtcttccttctctctctttcctctctctctataacaactccatcaacggttctctctccggagacgagtttaaatcgtgtcggaatctcagccatctcgatctgtcggagaatctcttcgtcgggtccatcccggagtcactcccttccacgcttccgaacctccgacgcttcgaaatctccgggaacaacttatcagacacgatcccggcgagcttcggagagttccgcaagctcgagtcgttgaatctcgccgggaatctcctctccggcaccatccccgcctctctcggcaacgtatccactctgagagaactcagactcgcttacaatccgttctctccgagtcgaatcccgagtcagctcggtaatctaacggagctctgtgtcctctggctcgccggctgcaacctcgtgggaccggtgcctcaaactctctccggtttgactcggttggttagtttggatttgacaacgaatcgactcaccgggtccgtcccgagctggatcacggagttgagaagcgtcgagcagatcgagatattcaacaactcgttctcgggggagttgccggaggcgatgggtaacatgacgatgctgaagagcttcgacgcgtccacgaacaggctgacggggaagattcccgacggcttggctcggttaaacctcgagtcgctgaacctcttcgagaacatgctcgaaggggccttgccggagagcatcacttgctccaaaacgttaaccgaactcaagctgttcaacaacaaactcaccggaacgataccgagtcagctcggcgcggactccatgttacagtacgtggacttttcgtataatcagttttccggcgagataccggcgaatctatgcggaggagggaaactagagtatctcatgcttatagacaactcgttttccggtgaaataccgaataatctgggaaagtgcaggagcttgacacgtgtccggttaagtaaaaacaaactttccggtcatgttccggaagagttctggggactgcctcgcttgtcgctgctcgaactctcggaaaattcgttcaccggaaaaatctctgaggccatcgccggtgcaaagaatctctcgaacctgaggatctcgaagaatcagttctcaggttccatccccggcgacatcggatcacttaacggactgatcgagatcgccggaggttctaacaacttctccggcgatattcctagcggtttggtgaaattgaagcaactgagcagactcagtctcagctataatcaactctccggcgagattccacgtggaatccgtgcttggaagaatctgaatgagctcaatttagctaataatcatctctccggcaaagtccctagagaactcggagacttgcctgttcttaactacctcgatctctcaaacaatctattttccggtgagatcccaccggagctccagactctgaagctcaacgttataaatttgtcgtataatcatctctccgggagagtacctcctctctacgctaacaaaaactacgcgtccagctttgtagggaatcatgacttgtgcttcgatgatcacgacagtgtttgtcggaagatcacacggtctaaaaacatcggttacgtctggattctcctatcgatattcatactcgcctgtcttgttttcgtcgttggggttgttatgtttatcgccaactgcaagaagatgagagcctccaagagcgctagcgcgtctaagtggagatccttccacaagcttcatttcagcgagcatgagatcgttgattgtcttgacgaacggaacgttattggttccggttcctccggtaaggtctacagagcggagcttagtggtggagaagtcgtagccgtgaagaaactaaacaaaacggctaaaggaggtgaagacgaatacagcgtttcgctgaatagagacgttttcgctgcggaggttgaaacgcttggaacgattaggcataagaatattgtgcgtttgtggtgttgctgcagctccggggactgcaagttgttggtatacgagtacatgcctaatgggagcttagctgatgcgttgcacagccaccgcaaaggccgggtggtattgggttggcccgaaaggttaaagatcgctttagacgcagctgagggtttgtcatacttacaccacgattgtgttcctcccatcgtgcaccgtgacgtgaagtctagtaacatattgctagatggagattatggagccaaagtagctgactttggcatcgctaaaatcggtcaaataagcggtgctaaatcgccagaggctatgtctgggatcgctggttcttgcggttacattgcaccaggtataaccaattcaaatgacaaaaccataagagttacataatgttttatgttattggtgcgttaattgttttttctaatgtttttgcagaatacgtatatacacttaaggtgaatgaaaagagcgatatctatagttttggtgtagtgattttggagttggtaacggggaagcaaccaacagatccagaacttggagataaagatatggtgaaatgggtgtgcactacgcttgaccaatgcggtttagaatcggtgatcgatcccaaacttgatctggggttcgaagaagagattagcaaagtcattcacattggtcttctttgtacgagtcctctccctctaaaccgaccttcgatgagaaaagttgtgattatgcttcaagaagtctctggtggtgtttctagtagcggtccaaatgcgtctaaacgctctaagagcagtgggaaactctcgccttactacgtggaagacgtaaacagcgt

SEQ ID NO: 23 - DNA, 2991 bp, Brassica juncea
  source 1..2991: mol_type = other DNA; note = BjuBHAE cDNA
atgctttattgtcttattctcctcctcctcctatgcctctcttccacgtatctatgtctctctctaaaccacgatgccaccattctccggcaagctaaactgggcttatccgacccggcccagtcactatcttcgtggtccgaaaacgacgtcacaccgtgccactggcgtggcatcacctgcgacgccacgtcagctgtcttctccgtcgatctctccagcttcacgctcctcggtcctttcccctccgtcctctgccgtcttccttctctctctttcctctctctctataacaactccatcaacggttctctctccggagacgagtttaaatcgtgtcggaatctcagccatctcgatctgtcggagaatctcttcgtcgggtccatcccggagtcactcccttccacgcttccgaacctccgacgcttcgaaatctccgggaacaacttatcagacacgatcccggcgagcttcggagagttccgcaagctcgagtcgttgaatctcgccgggaatctcctctccggcaccatccccgcctctctcggcaacgtatccactctgagagaactcagactcgcttacaatccgttctctccgagtcgaatcccgagtcagctcggtaatctaacggagctctgtgtcctctggctcgccggctgcaacctcgtgggaccggtgcctcaaactctctccggtttgactcggttggttagtttggatttgacaacgaatcgactcaccgggtccgtcccgagctggatcacggagttgagaagcgtcgagcagatcgagatattcaacaactcgttctcgggggagttgccggaggcgatgggtaacatgacgatgctgaagagcttcgacgcgtccacgaacaggctgacggggaagattcccgacggcttggctcggttaaacctcgagtcgctgaacctcttcgagaacatgctcgaaggggccttgccggagagcatcacttgctccaaaacgttaaccgaactcaagctgttcaacaacaaactcaccggaacgataccgagtcagctcggcgcggactccatgttacagtacgtggacttttcgtataatcagttttccggcgagataccggcgaatctatgcggaggagggaaactagagtatctcatgcttatagacaactcgttttccggtgaaataccgaataatctgggaaagtgcaggagcttgacacgtgtccggttaagtaaaaacaaactttccggtcatgttccggaagagttctggggactgcctcgcttgtcgctgctcgaactctcggaaaattcgttcaccggaaaaatctctgaggccatcgccggtgcaaagaatctctcgaacctgaggatctcgaagaatcagttctcaggttccatccccggcgacatcggatcacttaacggactgatcgagatcgccggaggttctaacaacttctccggcgatattcctagcggtttggtgaaattgaagcaactgagcagactcagtctcagctataatcaactctccggcgagattccacgtggaatccgtgcttggaagaatctgaatgagctcaatttagctaataatcatctctccggcaaagtccctagagaactcggagacttgcctgttcttaactacctcgatctctcaaacaatctattttccggtgagatcccaccggagctccagactctgaagctcaacgttataaatttgtcgtataatcatctctccgggagagtacctcctctctacgctaacaaaaactacgcgtccagctttgtagggaatcatgacttgtgcttcgatgatcacgacagtgtttgtcggaagatcacacggtctaaaaacatcggttacgtctggattctcctatcgatattcatactcgcctgtcttgttttcgtcgttggggttgttatgtttatcgccaactgcaagaagatgagagcctccaagagcgctagcgcgtctaagtggagatccttccacaagcttcatttcagcgagcatgagatcgttgattgtcttgacgaacggaacgttattggttccggttcctccggtaaggtctacagagcggagcttagtggtggagaagtcgtagccgtgaagaaactaaacaaaacggctaaaggaggtgaagacgaatacagcgtttcgctgaatagagacgttttcgctgcggaggttgaaacgcttggaacgattaggcataagaatattgtgcgtttgtggtgttgctgcagctccggggactgcaagttgttggtatacgagtacatgcctaatgggagcttagctgatgcgttgcacagccaccgcaaaggccgggtggtattgggttggcccgaaaggttaaagatcgctttagacgcagctgagggtttgtcatacttacaccacgattgtgttcctcccatcgtgcaccgtgacgtgaagtctagtaacatattgctagatggagattatggagccaaagtagctgactttggcatcgctaaaatcggtcaaataagcggtgctaaatcgccagaggctatgtctgggatcgctggttcttgcggttacattgcaccagaatacgtatatacacttaaggtgaatgaaaagagcgatatctatagttttggtgtagtgattttggagttggtaacggggaagcaaccaacagatccagaacttggagataaagatatggtgaaatgggtgtgcactacgcttgaccaatgcggtttagaatcggtgatcgatcccaaacttgatctggggttcgaagaagagattagcaaagtcattcacattggtcttctttgtacgagtcctctccctctaaaccgaccttcgatgagaaaagttgtgattatgcttcaagaagtctctggtggtgtttctagtagcggtccaaatgcgtctaaacgctctaagagcagtgggaaactctcgccttactacgtggaagacgtaaacagcgtt

SEQ ID NO: 24 - protein, 997 aa, Brassica juncea
  source 1..997: note = BjuBHAE aa
MLYCLILLLLLCLSSTYLCLSLNHDATILRQAKLGLSDPAQSLSSWSENDVTPCHWRGITCDATSAVFSVDLSSFTLLGPFPSVLCRLPSLSFLSLYNNSINGSLSGDEFKSCRNLSHLDLSENLFVGSIPESLPSTLPNLRRFEISGNNLSDTIPASFGEFRKLESLNLAGNLLSGTIPASLGNVSTLRELRLAYNPFSPSRIPSQLGNLTELCVLWLAGCNLVGPVPQTLSGLTRLVSLDLTTNRLTGSVPSWITELRSVEQIEIFNNSFSGELPEAMGNMTMLKSFDASTNRLTGKIPDGLARLNLESLNLFENMLEGALPESITCSKTLTELKLFNNKLTGTIPSQLGADSMLQYVDFSYNQFSGEIPANLCGGGKLEYLMLIDNSFSGEIPNNLGKCRSLTRVRLSKNKLSGHVPEEFWGLPRLSLLELSENSFTGKISEAIAGAKNLSNLRISKNQFSGSIPGDIGSLNGLIEIAGGSNNFSGDIPSGLVKLKQLSRLSLSYNQLSGEIPRGIRAWKNLNELNLANNHLSGKVPRELGDLPVLNYLDLSNNLFSGEIPPELQTLKLNVINLSYNHLSGRVPPLYANKNYASSFVGNHDLCFDDHDSVCRKITRSKNIGYVWILLSIFILACLVFVVGVVMFIANCKKMRASKSASASKWRSFHKLHFSEHEIVDCLDERNVIGSGSSGKVYRAELSGGEVVAVKKLNKTAKGGEDEYSVSLNRDVFAAEVETLGTIRHKNIVRLWCCCSSGDCKLLVYEYMPNGSLADALHSHRKGRVVLGWPERLKIALDAAEGLSYLHHDCVPPIVHRDVKSSNILLDGDYGAKVADFGIAKIGQISGAKSPEAMSGIAGSCGYIAPEYVYTLKVNEKSDIYSFGVVILELVTGKQPTDPELGDKDMVKWVCTTLDQCGLESVIDPKLDLGFEEEISKVIHIGLLCTSPLPLNRPSMRKVVIMLQEVSGGVSSSGPNASKRSKSSGKLSPYYVEDVNSV

SEQ ID NO: 25 - DNA, 3260 bp, Brassica juncea
  source 1..3260: mol_type = genomic DNA; note = BjuAHSL2 gene
atgctttattatcttattctcctcctcctatgcctctcttctacgtacctatgcctctcaccaaaccacgatgccaccatcctccggcaagccaaactgggcttatccgacccggcccagtcactatcttcctggtccgaaaacgacgtcactccgtgccactggcgcggcatcacctgcgacgccacgtcagctgtcttctccgttaatctctccagcttcatgctcctcggtcctttcccctccgtcctctgccgtcttccttcgctgtctttcctctctctctacaacaactccatcaacggttccctctccggcgatgagtttacatcgtgtcggaatctcagccaactcgatttgtcggagaatctcttggttggttccatcccggagtcacttcctttcacgctgccgaacctccgacacttcgaagtttccgggaacaacttatcagatacgattccagcgagcttcggagagttccgcaagctcgagaagttaaacctcgccggaaacctcctctccggtaccattcccgcctctctcggaaacctatccactctgagagaactcaaactcgcttacaatctgttttcaccgggtcgaatcccgagtcaactcggtaatctaacggagcttcgtgttctctggctcgctggctgcaacctcgctggtccggtgcctcaagctctgtcccggttgactcacttggtcagcttggatttgacgatgaatcaactcaccgggtctgtcctgagttggatcacggagctgaaaagcatcgagcaaatcgagatattcaacaactcgttctcaggcgagttaccggaggcgatgggtaacatgacgatgctaaagaatttcgacgcgtcgacgaacaagctgacggggaagatccccgagggcttggctcggttaaacctcgagtctctcaacctcttcgagaacatgctcgaagggcccttgccggagagcataactcactccaaaacgttaacggaactaaaactgttcaacaacaaactcaccggggagataccgagtcaactcggcgcgagctccccgttgcagttcgtggacttttcgtataatcagttttccggcgagataccggcgaatatatgcggaggagggaagctagagtttctcatgcttatagggaactctttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcgatctcagctctaatcagctctccggcgagattccacgtggaattcgtggttggaagaatctgaacgagctcaatttagctaataaccatctctccggcgaagtctctagagagctcggagacttgccggttcttaactacctcgatctctcaaacaatcaattttccggtgagattccaccggagctccagaatctgaagctcaacgttataaacctatcgtataatcatctctccgggagagtgcctcctctctacgctaacaaaatctacgattctagctttgtcggaaaccatgacttgtgcgtcgatgatcacgacagtctttgtcggaagaagagcacacggtctcaaaacatcggttatgtctggattctcctatcgattttcatactcacctgtatggtttttgttgttggggttgttatgtttatcgccaactgcaagaagatgagagcatccaagagcgctagattctccgcgtccaagtggagatccttccacaagcttcatttcagcgagcatgagatcgttgattgccttgacgaacggaacgtgatcggatctggttcctccggtaaagtctatagagtggagcttagtggcggagaagtagtagtagctgtgaagaaactaaacaaaacggctaaaggaggtgaagacgattcgttgaacagagacgttttcgctgtggaggttgaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttcctccgatcgtgcaccgtgacgtgaagtctagtaacatattgctagatgcagattatggagccaaagtagctgactttggcattgctaaaatcggtcaaatgagcggtgctaaatcgccagaagctatgtctggaatcgccggttcttgcggttacattgcaccaggtataaccaaatcaaatgataaaaagataacttatatttcaaatgttatttgtgctttatagctcttttttatgctgttttgtagaatacgtatatacacttagggtgaatgaaaagagtgatatctacagttttggtatcgtgcttttggagttggtaacggggaagcaaccaacagatctagaacttggagataaagatatggtgaagtgggtgtgctctacccttgaccaatgcggtttagaatcggtgatcgatcccaaacttgatctggggttcaaagaagagattagcaaagtcattcacattggtcttctttgtacgagtcctctccctctaaaccgaccttccatgagaaaagttgtgatcatgcttcaagaagtctctggtgctgtttctagtagcagaccaaatgcgtccaaacgctctaagagcagtgggaaactctcgccttactatgtggaagacgtaaacagcgtttgagcattcaaatatgggccggtatgggttgggctgggccatatgtattaattgggttgtatagttaggtgaattgtaagaaatcagaaacagaaaagtggacctttctgattaggtaatggcttttaatgctatgatgtttggatattaaaaatatttttaattaacgcgatgattgagaggg

SEQ ID NO: 26 - DNA, 2994 bp, Brassica juncea; identical to SEQ ID NO: 20
  source 1..2994: mol_type = other DNA; note = BjuAHSL2 cDNA

SEQ ID NO: 27 - protein, 988 aa, Brassica juncea
  source 1..988: note = BjuAHSL2 aa
MTTITTSLFLSLVLSCVLQVSSNGDAEILSRVKRSRLSDPDGKLNDWIITGDNRNPCNWTGIKCDSKNRSVTAIDLSGYGLSGGFPYEFCRVRTLVNITLSENNLNGTIDSAPLSLCSRIHVLILNENGFSGKLPEFSPEFRSLRVIELESNLFTGEIPESYGKLTSLRVLNLNGNSLGGIVPAFLGNLTELTRLELAYVGFEPGPIPSTFGSLTKLSNLRLTNSNLVGEIPDSIGNLVSLENLDLAMNGLSGEIPESIGKLKSVYQLELYDNNLSGKLPESIGNLTAMRNFDVSQNNLTGELPEKIASLQLVSFNLNDNFFNGELPRVVALNPNLVEFKIFNNSFTGTLPRSFGKFSGVTEFDVSTNRFSGELPPYLCYMKKLERLISFGNRISGEIPESYGECGSLNYIRTADNKLSGEVPVKLWELPLTRLELSNNQLQGSIPPSISNARNLSQLEISGNNFSGAIPGRICDLGELRVVDLSRNRFSGSLPSCINRLKNLERVEMQENMLDGEIPSSLSSCSKLTELKLSDNRLRGGIPPELGELPVLNYLDLSNNQLSGEIPPDLLKLKLNQFDVSDNRLSGKIPSGFQQDIFLRSFLGNPDLCAPDMDPIRPCRSKPDPRYILVISVVCIVVLIGALVWLFIKTKPLFQRKPNRTDKVTIFQRVGFTEEDIYPQLTEDNIIGSGGSGLVYRVTLKSGETLAVKKLWGGPGRKPESETVFRSEVEILGRVRHGNIVKLLMCCSGEEFRFLVYEFMENGSLGDVLHSEKEHRAVSPLDWTTRFSIALGAAQGLAYLHHDSVPAIVHRDVKSNNILLDHEMKPRVADFGLAKPLKREDNNSVSDVSPMSCVAGSYGYIAPEYGYTLRVNEKSDVYSFGVVLLELITGKRPNDSSFGENKDIVKYAMESALCYSSPSPGDKAMNQDSPGNFRDLSKLVDPKMELSRGEYEEVEKVLEIALLCTSSFPISRPTMRKVVELLKEKKPFE

SEQ ID NO: 28 - DNA, 3320 bp, Brassica juncea
  source 1..3320: mol_type = genomic DNA; note = BjuBHSL2 gene
atgctttattgtcttattctcctcctcctcctatgcctctcttccacgtatctatgtctctctctaaaccacgatgccaccattctccggcaagctaaactgggcttatccgacccggcccagtcactatcttcgtggtccgaaaacgacgtcacaccgtgccactggcgtggcatcacctgcgacgccacgtcagctgtcttctccgtcgatctctccagcttcacgctcctcggtcctttcccctccgtcctctgccgtcttccttctctctctttcctctctctctataacaactccatcaacggttctctctccggagacgagtttaaatcgtgtcggaatctcagccatctcgatctgtcggagaatctcttcgtcgggtccatcccggagtcactcccttccacgcttccgaacctccgacgcttcgaaatctccgggaacaacttatcagacacgatcccggcgagcttcggagagttccgcaagctcgagtcgttgaatctcgccgggaatctcctctccggcaccatccccgcctctctcggcaacgtatccactctgagagaactcagactcgcttacaatccgttctctccgagtcgaatcccgagtcagctcggtaatctaacggagctctgtgtcctctggctcgccggctgcaacctcgtgggaccggtgcctcaaactctctccggtttgactcggttggttagtttggatttgacaacgaatcgactcaccgggtccgtcccgagctggatcacggagttgagaagcgtcgagcagatcgagatattcaacaactcgttctcgggggagttgccggaggcgatgggtaacatgacgatgctgaagagcttcgacgcgtccacgaacaggctgacggggaagattcccgacggcttggctcggttaaacctcgagtcgctgaacctcttcgagaacatgctcgaaggggccttgccggagagcatcacttgctccaaaacgttaaccgaactcaagctgttcaacaacaaactcaccggaacgataccgagtcagctcggcgcggactccatgttacagtacgtggacttttcgtataatcagttttccggcgagataccggcgaatctatgcggaggagggaaactagagtatctcatgcttatagacaactcgttttccggtgaaataccgaataatctgggaaagtgcaggagcttgacacgtgtccggttaagtaaaaacaaactttccggtcatgttccggaagagttctggggactgcctcgcttgtcgctgctcgaactctcggaaaattcgttcaccggaaaaatctctgaggccatcgccggtgcaaagaatctctcgaacctgaggatctcgaagaatcagttctcaggttccatccccggcgacatcggatcacttaacggactgatcgagatcgccggaggttctaacaacttctccggcgatattcctagcggtttggtgaaattgaagcaactgagcagactcagtctcagctataatcaactctccggcgagattccacgtggaatccgtgcttggaagaatctgaatgagctcaatttagctaataatcatctctccggcaaagtccctagagaactcggagacttgcctgttcttaactacctcgatctctcaaacaatctattttccggtgagatcccaccggagctccagactctgaagctcaacgttataaatttgtcgtataatcatctctccgggagagtacctcctctctacgctaacaaaaactacgcgtccagctttgtagggaatcatgacttgtgcttcgatgatcacgacagtgtttgtcggaagatcacacggtctaaaaacatcggttacgtctggattctcctatcgatattcatactcgcctgtcttgttttcgtcgttggggttgttatgtttatcgccaactgcaagaagatgagagcctccaagagcgctagcgcgtctaagtggagatccttccacaagcttcatttcagcgagcatgagatcgttgattgtcttgacgaacggaacgttattggttccggttcctccggtaaggtctacagagcggagcttagtggtggagaagtcgtagccgtgaagaaactaaacaaaacggctaaaggaggtgaagacgaatacagcgtttcgctgaatagagacgttttcgctgcggaggttgaaacgcttggaacgattaggcataagaatattgtgcgtttgtggtgttgctgcagctccggggactgcaagttgttggtatacgagtacatgcctaatgggagcttagctgatgcgttgcacagccaccgcaaaggccgggtggtattgggttggcccgaaaggttaaagatcgctttagacgcagctgagggtttgtcatacttacaccacgattgtgttcctcccatcgtgcaccgtgacgtgaagtctagtaacatattgctagatggagattatggagccaaagtagctgactttggcatcgctaaaatcggtcaaataagcggtgctaaatcgccagaggctatgtctgggatcgctggttcttgcggttacattgcaccaggtataaccaattcaaatgacaaaaccataagagttacataatgttttatgttattggtgcgttaattgttttttctaatgtttttgcagaatacgtatatacacttaaggtgaatgaaaagagcgatatctatagttttggtgtagtgattttggagttggtaacggggaagcaaccaacagatccagaacttggagataaagatatggtgaaatgggtgtgcactacgcttgaccaatgcggtttagaatcggtgatcgatcccaaacttgatctggggttcgaagaagagattagcaaagtcattcacattggtcttctttgtacgagtcctctccctctaaaccgaccttcgatgagaaaagttgtgattatgcttcaagaagtctctggtggtgtttctagtagcggtccaaatgcgtctaaacgctctaagagcagtgggaaactctcgccttactacgtggaagacgtaaacagcgtttgagtattcaaatatgggccggtatgggttgggttgggccagaagtagtgatttggtttgtgctaagttagatgaattgtaagaagtcagaaacagaaaagtggagctttctgattaggtagctcttaatgttaagaagtttggatattaaaatattttgaattaacgcaatgattgtaacatttgatggttggtccacactatagccaacctttatttgtaaacgaatttgcattataa

SEQ ID NO: 29 - DNA, 2596 bp, Brassica juncea
  source 1..2596: mol_type = other DNA; note = BjuBHSL2 cDNA
atgctttattatcttattctcctcctcctatgcctctcttctacgtacctatgcctctcaccaaaccacgatgccaccatcctccggcaagccaaactgggcttatccgacccggcccagtcactatcttcctggtccgaaaacgacgtcactccgtgccactggcgcggcatcacctgcgacgccacgtcagctgtcttctccgttaatctctccagcttcatgctcctcggtcctttcccctccgtcctctgccgtcttccttcgctgtctttcctctctctctacaacaactccatcaacggttccctctccggcgatgagtttacatcgtgtcggaatctcagccaactcgatttgtcggagaatctcttggttggttccatcccggagtcacttcctttcacgctgccgaacctccgacacttcgaagtttccgggaacaacttatcagatacgattccagcgagcttcggagagttccgcaagctcgagaagttaaacctcgccggaaacctcctctccggtaccattcccgcctctctcggaaacctatccactctgagagaactcaaactcgcttacaatctgttttcaccgggtcgaatcccgagtcaactcggtaatctaacggagcttcgtgttctctggctcgctggctgcaacctcgctggtccggtgcctcaagctctgtcccggttgactcacttggtcagcttggatttgacgatgaatcaactcaccgggtctgtcctgagttggatcacggagctgaaaagcatcgagcaaatcgagatattcaacaactcgttctcaggcgagttaccggaggcgatgggtaacatgacgatgctaaagaatttcgacgcgtcgacgaacaagctgacggggaagatccccgagggcttggctcggttaaacctcgagtctctcaacctcttcgagaacatgctcgaagggcccttgccggagagcataactcactccaaaacgttaacggaactaaaactgttcaacaacaaactcaccggggagataccgagtcaactcggcgcgagctccccgttgcagttcgtggacttttcgtataatcagttttccggcgagataccggcgaatatatgcggaggagggaagctagagtttctcatgcttatagggaactctttttccggtgaaataccgataaatcttggaaagtgcaggagcttgacacgtgtcaggctgaataataacaagctttctggtcacgttccggaagagttctgggggttgcctcgtatgtcgctgctcgaactctcggagaactcgttcaccggaagaatctctgagtccatcgccggtgcaaagaatttatctaacctgagaatctcaaagaatcagttctcaggttcgattcccggcgaaatcggatcgcttaacggactcatcgagatcaccggagatgaaaatagcttctccggcgaggttcctagtactttggtgaagcttaagcaactgagcaggctcgatctcagctctaatcagctctccggcgagattccacgtggaattcgtggttggaagaatctgaacgagctcaatttagctaataaccatctctccggcgaagtctctagagagctcggagacttgccggttcttaactacctcgatctctcaaacaatcaattttccggtgagattccaccggagctccagaatctgaagctcaacgttataaacctatcgtataatcatctctccgggagagtgcctcctctctacgctaacaaaatctacgattctagctttgtcggaaaccatgacttgtgcgtcgatgatcacgacagtctttgtcggaagaagagcacacggtctcaaaacatcggttatgtctggattctcctatcgattttcatactcacctgtatggtttttgttgttggggttgttatgtttatcgccaactgcaagaagatgagagcatccaagagcgctagattctccgcgtccaagtggagatccttccacaagcttcatttcagcgagcatgagatcgttgattgccttgacgaacggaacgtgatcggatctggttcctccggtaaagtctatagagtggagcttagtggcggagaagtagtagtagctgtgaagaaactaaacaaaacggctaaaggaggtgaagacgattcgttgaacagagacgttttcgctgtggaggttgaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttcctccgatcgtgcaccgtgacgtgaagtctagtaacatattgctagatgcagattatggagccaaagtagctgactttggcattgctaaaatcggtcaaatgagcggtgctaaatcgccagaagctatgtctggaatcgccggttcttgcggttacattgcaccag

SEQ ID NO: 30 - protein, 988 aa, Brassica juncea; identical to SEQ ID NO: 27
  source 1..988: note = BjuBHSL2 aa

SEQ ID NO: 31 - DNA, 3085 bp, Brassica nigra
  source 1..3085: mol_type = genomic DNA; note = BniBHAE gene
atgctttattgtcttattctcctcctcctatgcctctcttccacgtacccatgtctctctctaaaccacgatgccaccatcctccggcaagctaaactgggcttatccgacccggcccagtcactatcttcctggtccgaaaacgacgtcacaccgtgccactggcgaggcatcacctgcgacgccacgtcagctgtcatctccgtcgatctctccagcttcacgctcgtcggtcctttcccctccgtcctctgccgtcttccttctctctctttcctctctctctataacaactccatcaacggttctctctccggagacgagtttaaatcgtgtcggaacctcagccatctcgatctgtcggagaatctcttcgtcgggtccatcccggagtcactccctctcacccttccgaacctccggcgcttcgaaatctccgggaacaacttatcagacacgatcccggcgagcttcggagagttccgcaagctcgagtcgttgaatctcgccgggaatctcctctccggtaccatccccgcctctctcggcaacgtatccactctgagagagctcagactcgcttacaatccgttctctccgagtcgaatcccgagtcagctcggtaatctaacggagctccgtgtcctctggctcgccggctgcaacctcgtgggaccggtgcctcaaactctctccggtttgactcggttggttagtttggatttgacaacgaatcgactcaccgggtccgtcccgagctggatcacggagctgaaaagcgtcgagcagatcgagatattcaacaactcgttctcgggggagttaccggatgcgatgggtaacatgacgatgctaaagagcttcgacgcgtcgacgaacaagctgacggggaggatccccgacggcttggctcggttaaacctcgagtcgctgaacctcttcgagaacatgctcgaagggcccttgccggagagcatcactcgctccaaaacgttaacggaactcaagctgttcaacaacagactcaccggagagataccgagtcaactcggagcgaactccatgttacagtacgtggacttttcgtataatcagttttccggcgagataccggcgaatctatgcggaggagggaagctagagtatctcatgcttatagacaactcgttttccggtgaaataccgaataatctgggaaagtgcaggagcttgacacgtgtccggttaagtaaaaacaagctttccggtcatgttccggaagagttctggggactgcctcgcttgtcgctgctcgaactctcggaaaatttgttcagtggaacgatctctgaggccatcgccggtgcaaagaatctctcaaacctgaggatctcgaagaatcagttctcaggttccatccccgtcgaaatcggatcacttaacggactgatcgagattgccggaggttctaacaacttctccggcgatattcctagtggtttggtgaagcttaagcaactgagcagactcagtctcagctataatcaactctccggcgagattccacgtggaatccgtgcttggaagaatctgaacgagcttaatttagctagtaatcatctctcaggcgaagtccctagagaactcggagacttgcctgttcttaactacctcgacctctcaaacaatcaattttccggtgagattccaccggagctgcagagtctgaagctcaacgtcataaatctgtcttataatcatctctccgggagagtacctcctctctacgctaacgaaaactacgcgtccagctttctcgggaatcatgacttgtgcttcgatgatcacgacagtgtttgtcggaagatcacacggtctaaaaacatcggttacgtctggattctcctatcgattttcatactcgcctgtcttgtttttcttgttggggttgttatgtttatcgccaactgcaagaagatgagagcctccaagagcgctagactctccgcgtctaagtggagatccttccacaagcttcatttcagcgagcatgagatcgttgattgtcttgacgaacggaacgttattggttccggttcctccggtaaggtctacagagcggagcttagtggtggagaagtcgtggcggtgaagaaactcaacaaaacggctaaaggaggtgaagacgaatacagcgtttcgttgaatagagacgtcttcgctgcggaggttgaaacgcttggaacgattaggcacaagagtattgtgcgtttgtggtgttgctgcagctccggggactgcaagttgttggtatatgagtacatgcctaatgggagcttagctgatgcgttgcacagccaccgcaaaggtggagtgttgttgggttggcctgaaaggttaaggatcgctttagacgcagctgagggtttgtcatacttacaccacgattgtgttcctccgatcgtgcaccgtgacgtgaagtctagtaacatattgctagatggagattatggagccaaagtagctgactttggtatcgctaaagtcggtcaaataagcggtgctaaatcgcaagaggctatgtctgggatcgctggttcttgcggttacattgcaccaggtataaccaattcaaatgataaagccataagagttacataatgtttaatgtaattggtgcgttaattgttttttctaatgtttttgcagaatacgtatacacacttaaggtgaatgaaaagagcgatatctacagttttggtgtagtgattttggagttggtaacggggaagcaaccaacagatcaagaactcggagataaagatattgtgaagtgggtgtgtactacgcttgaccaatgcggtttagaattggtgatcgatcccaaacttgatatgggtttcaaagaagagattagcaaagtcattcacattggtcttctctgtacgagtcctctccctctaaaccgaccttccatgagaaaagttgtgatcatgcttcaagaagtctctggtggtgtttctagtagcggtccaaatgcgtctaaacgctctaagagcagtgggaaactttcgccttactacgtggaagacgtaaacagcgtttg

SEQ ID NO: 32 - DNA, 2996 bp, Brassica nigra
  source 1..2996: mol_type = other DNA; note = BniBHAE cDNA
atgctttattgtcttattctcctcctcctatgcctctcttccacgtacccatgtctctctctaaaccacgatgccaccatcctccggcaagctaaactgggcttatccgacccggcccagtcactatcttcctggtccgaaaacgacgtcacaccgtgccactggcgaggcatcacctgcgacgccacgtcagctgtcatctccgtcgatctctccagcttcacgctcgtcggtcctttcccctccgtcctctgccgtcttccttctctctctttcctctctctctataacaactccatcaacggttctctctccggagacgagtttaaatcgtgtcggaacctcagccatctcgatctgtcggagaatctttcgtcgggtccatcccggagtcactccctctcacccttccgaacctccggcgcttcgaaatctccgggaacaacttatcagacacgatcccggcgagcttcggagagttccgcaagctcgagtcgttgaatctcgccgggaatctcctctccggtaccatccccgcctctctcggcaacgtatccactctgagagagctcagactcgcttacaatccgttctctccgagtcgaatcccgagtcagctcggtaatctaacggagctccgtgtcctctggctcgccggctgcaacctcgtgggaccggtgcctcaaactctctccggtttgactcggttggttagtttggatttgacaacgaatcgactcaccgggtccgtcccgagctggatcacggagctgaaaagcgtcgagcagatcgagatattcaacaactcgttctcgggggagttaccggatgcgatgggtaacatgacgatgctaaagagcttcgacgcgtcgacgaacaagctgacggggaggatccccgacggcttggctcggttaaacctcgagtcgctgaacctcttcgagaacatgctcgaagggcccttgccggagagcatcactcgctccaaaacgttaacggaactcaagctgttcaacaacagactcaccggagagataccgagtcaactcggagcgaactccatgttacagtacgtggacttttcgtataatcagttttccggcgagataccggcgaatctatgcggaggagggaagctagagtatctcatgcttatagacaactcgttttccggtgaaataccgaataatctgggaaagtgcaggagcttgacacgtgtccggttaagtaaaaacaagctttccggtcatgttccggaagagttctggggactgcctcgcttgtcgctgctcgaactctcggaaaatttgttcagtggaacgatctctgaggccatcgccggtgcaaagaatctctcaaacctgaggatctcgaagaatcagttctcaggttccatccccgtcgaaatcggatcacttaacggactgatcgagattgccggaggttctaacaacttctccggcgatattcctagtggtttggtgaagcttaagcaactgagcagactcagtctcagctataatcaactctccggcgagattccacgtggaatccgtgcttggaagaatctgaacgagcttaatttagctagtaatcatctctcaggcgaagtccctagagaactcggagacttgcctgttcttaactacctcgacctctcaaacaatcaattttccggtgagattccaccggagctgcagagtctgaagctcaacgtcataaatctgtcttataatcatctctccgggagagtacctcctctctacgctaacgaaaactacgcgtccagctttctcgggaatcatgacttgtgcttcgatgatcacgacagtgtttgtcggaagatcacacggtctaaaaacatcggttacgtctggattctcctatcgattttcatactcgcctgtcttgtttttcttgttggggttgttatgtttatcgccaactgcaagaagatgagagcctccaagagcgctagactctccgcgtctaagtggagatccttccacaagcttcatttcagcgagcatgagatcgttgattgtcttgacgaacggaacgttattggttccggttcctccggtaaggtctacagagcggagcttagtggtggagaagtcgtggcggtgaagaaactcaacaaaacggctaaaggaggtgaagacgaatacagcgtttcgttgaatagagacgtcttcgctgcggaggttgaaacgcttggaacgattaggcacaagagtattgtgcgtttgtggtgttgctgcagctccggggactgcaagttgttggtatatgagtacatgcctaatgggagcttagctgatgcgttgcacagccaccgcaaaggtggagtgttgttgggttggcctgaaaggttaaggatcgctttagacgcagctgagggtttgtcatacttacaccacgattgtgttcctccgatcgtgcaccgtgacgtgaagtctagtaacatattgctagatggagattatggagccaaagtagctgactttggtatcgctaaagtcggtcaaataagcggtgctaaatcgcaagaggctatgtctgggatcgctggttcttgcggttacattgcaccagaatacgtatacacacttaaggtgaatgaaaagagcgatatctacagttttggtgtagtgattttggagttggtaacggggaagcaaccaacagatcaagaactcggagataaagatattgtgaagtgggtgtgtactacgcttgaccaatgcggtttagaattggtgatcgatcccaaacttgatatgggtttcaaagaagagattagcaaagtcattcacattggtcttctctgtacgagtcctctccctctaaaccgaccttccatgagaaaagttgtgatcatgcttcaagaagtctctggtggtgtttctagtagcggtccaaatgcgtctaaacgctctaagagcagtgggaaactttcgccttactacgtggaagacgtaaacagcgtttga

SEQ ID NO: 33 - protein, 997 aa, Brassica nigra
  source 1..997: note = BniBHAEaa
MLYCLILLLLCLSSTYPCLSLNHDATILRQAKLGLSDPAQSLSSWSEDVTPCHWRGITCDATSAVISVDLSSFTLVGPFPSVLCRLPSLSFLSLYNNSINGSLSGDEFKSCRNLSHLDLSENLFVGSIPESLPLTLPNLRRFEISGNNLSDTIPASFGEFRKLESLNLAGNLLSGTIPASLGNVSTLRELRLAYNPFSPSRIPSQLGNLTELRVLWLAGCNLVGPVPQTLSGLTRLVSLDLTTNRLTGSVPSWITELKSVEQIEIFNNSFSGELPDAMGNMTMLKSFDASTNKLTGRIPDGLARLNLESLNLFENMLEGPLPESITRSKTLTELKLFNNRLTGEIPSQLGANSMLQYVDFSYNQFSGEIPANLCGGGKLEYLMLIDNSFSGEIPNNLGKCRSLTRVRLSKNKLSGHVPEEFWGLPRLSLLELSENLFSGTISEAIAGAKNLSNLRISKNQFSGSIPVEIGSLNGLIEIAGGSNNFSGDIPSGLVKLKQLSRLSLSYNQLSGEIPRGIRAWKNLNELNLASNHLSGEVPRELGDLPVLNYLDLSNNQFSGEIPPELQSLKLNVINLSYNHLSGRVPPLYANENYASSFLGNHDLCFDDHDSVCRKITRSKNIGYVWILLSIFILACLVFLVGVVMFIANCKKMRASKSARLSASKWRSFHKLHFSEHEIVDCLDERNVIGSGSSGKVYRAELSGGEVVAVKKLNKTAKGGEDEYSVSLNRDVFAAEVETLGTIRHKSIVRLWCCCSSGDCKLLVYEYMPNGSLADALHSHRKGGVLLGWPERLRIALDAAEGLSYLHHDCVPPIVHRDVKSSNILLDGDYGAKVADFGIAKVGQISGAKSQEAMSGIAGSCGYIAPEYVYTLKVNEKSDIYSFGVVILELVTGKQPTDQELGDKDIVKWVCTTLDQCGLELVIDPKLDMGFKEEISKVIHIGLLCTSPLPLNRPSMRKVVIMLQEVSGGVSSSGPNASKRSKSSGKLSPYYVEDVNSV

SEQ ID NO: 34 - DNA, 3064 bp, Brassica nigra
  source 1..3064: mol_type = genomic DNA; note = BniBHSL2 gene
atgaccaccaccattactactacttctctctttttgttacttgttctctcatgtgttctccaagcttcttccaacggagacgctgagatactgagcagagtcaaaaccagcagactttctgacccggatggaaagttaaatgattgggtcataaccggagataaccggagtccctgtaactggactggaatcacatgcgactccaaaaacggcgccgtcacggcaatagatctctccgactacggaatctccggtgggttcccgtacgggttctgccgtatacgtaccctcgtcaacatcactctctctaaaaacaatctcaacggtacgatagattcagctcctctcacactatgctctcgaatccacgtcctgatcctcacagaaaacagcttctccgggaagctaccggagttttcgccggagtttcgcaacctacgcgttctcgaactggagtccaactttttctccggcgagatccccgaaagctacggaaagcttccatctctgcaagttctgaatctcaacggcaactcgctcggcggaatcgttccggcgtttttgggtaacctgaccgagttgactcgcctcgagctcgcatacgttcagttcgagcctggtccgattccgtcgacgtttgggaacttgacgaagatgacttatctccggctaactaactcgaacatcgtcggagagattcctgattcgatcgggaatctggtttccctggtgaatctcgatttagctcagaacggtctctccggagagataccggagagcatcggaaagctaaaatcgatttatcaaatggtgctttacatcaatcagctatcgggaaaattgccggagagtatcggaaacttaacggctatgaggaatttcgacgtttcgcagaataatctctccggtgatttgcctgagacgatcgccgctctccaggtcgtttctttccatctcaatgataatttattcaccggtgaattgccacgtggcattgctttgaatcctaatctcgttgatttcaagatctttaacaacagtttcaccgggagtttacctacgagtttcggtaaattctccggtttaactgagttcgacgtctcgactaacagattctccggtgagttgcctccgtatctatgctacgggaagaaacttgaaaagctaatcattttcagcaatcagttaagcggcgagattccggaaacgtacggtgagtgcgacacgcttaattatattagaatggcggataacaaactctccggcgaagttccggttaagttttgggagcttcctctcactcgtctcgagctatctaacaaccgattagaaggttcgattcctccgtcgatatccaaagctcgtcagctctctcagctcgagatctccggaaacaagttgtccggtgcgattccggcgagaatctgtgatctcgaaggactcagggacgtcgatctcagccgtaaccgtttctcaggatctattccctcttgcattaaccgattgaagaatctggagagagtggagatgcaggagaacatgctcgacggcgagatcccgagttcggtgagttcatgcgccaagttaacggagttgaatctctccgataaccgtctccgcggcgaaattccaccggagctcggtgagttaccggttttgaactatctggatctctccaacaaccaactctccggtgagatcccggcggagttgttgaagctgaagctgaatctgttcaacgtctccgataacaaactctccgggaagataccttctggttttcaacaagatgtttttctaccgagttttttgggtaacccgggactctgtgctccggatatggatccgattcgaccttgccgatccaaacccgagccccggtttatcctggttatctccgtagtctgcatcgttgtactaatcggagctttggtttggctttttattaaaaccaaaccgttattccagagaaaaccgaaccggaccgataaagtaaccattttccagcggatcgggttcacagaggaagacatatacccgcaattaacagaagataacataatcgggtcgggcgggtcgggtttggtttacagagtgacactaaagtcaggccaaacgctcgcggtgaagaaactttggggtggaccgggtcaaaaacccgaatccgaatcggttttcagatccgaggtagagattctgggtcgggtcagacatggtaacatagtgaagcttctcatgtgttgcagcggcgaggagtttcggtttttagtgtacgagtatatggaaaacggcagcttgggcgacgttttgcattcagagaaagaacatcgtgccgtttctccgcttgattggacgacacggttttcgatcgcgcttggtgcagctcaaggacttgcttatctacatcatgactctgttccggcgattgttcaccgtgacgtcaaaagcaataatatattgttggaccatgagatgaagccacgtgttgctgatttcggtttagctaaaccgttgagaagagaagtcaataatggtgtctcggatgtttctcctatgtcttgtgttgctggatcctacggctacattgctccaggttcgaattctaaatgctaaaccgaaataaaccggaaaaccaaatatttattaatgttatttttattctgttgttgtgattgcacatgcagaatatggttatacgtcgagagtgaatgagaagagcgatgtgtatagttttggtgtcgtgttacttgagttgattacgagtaaaagaccaaatgattcgtcgtttggggagaataaagacattgttaagtttgcaatggagtcagctttgtcttactcttctccatcacctgaagacaaagctatgaatcaagattcacctggaaattgtcgagatcttagtaagcttgtggatccaaagatggaactttctaggggggagtatggagaggtagagaaagtttttgagattgcgctgctttgcacgtcgtcgtttccgataagtaggccgactatgaggaaagtagtagagctgctcaaagagaagaagccacttgagtga

SEQ ID NO: 35 - DNA, 2967 bp, Brassica nigra
  source 1..2967: mol_type = other DNA; note = BniBHSL2 cDNA
atgaccaccattactacttctctcttcttgtcacttgttctttcttgtgttctccaagtttcttccaatggagacgccgagatactgagccgagtcaaaaggagcaggctctctgacccggacggaaagttaaatgattgggtcataaccggagataaccggaatccctgtaactggacgggaattaaatgcgactccaaaaacggttccgtcacggcaattgatctctccggctacggactctccggtgggttcccatacgggttctgccgtatacgtaccctcgtcaacatcactctctccgaaaacaatctcaacggtacgatagattcagctcctctctccctatgttctcgaatccacgtcctgatcctcaacgaaaacggcttctccgggaagttaccggaattttcgccggcgtttcgtaacctccgcgtcatcgaactggaatccaacttgttcaccggcgagattcccgaaagctacggaaagcttacatctctccgagttctgaatctcaacggcaactcgctcggcggaatcgttccggcgtttttgggtaacctgaccgagttgactcgtctcgagctcgcgtacgtccgattcgagcccggtccgattccgtcgacgttcgggagcttgacgaagctgagtaacctccggctgactaagtcgaacctcgtcggcgagattcctgattcgatcgggaatctggtttcgttagagaatctcgatttagctatgaacggtctctccggagagataccggagagcatcggaaacctcaaatcggtttatcagttggagctttacgacaataatctatctggaaaattgccggagagtatcggaaacttaacggctatgaggaatttcgacgtttcgcagaataatctcaacggtgagttacctgagaaggtcgcttctctccagctcgtgtcgtttaatctcaacgataacttcttcaacggtgagttgccacgtgtcgtggcttcgaatcctaatctcgtcgagttcaagatcttcaacaacagcttcacagggacgttgcctaggagtttcggcaaattctcaggtgtaaccgagttcgacgtctcgacgaatagattctccggtgagttaccgccgtatctttgttacatgaagaaactcgagaggctaatcagtttcggcaatcggattagcggcgagattcctgaatcttacggcgagtgtgactcgcttaattatattcgaacggcggataacaaactctccggcgaagttccggttaaggtttgggagcttcctctcactcgtctcgagctatctaacaatcaattacaaggttcgattcctccgtcgatatccaaagctcgtaatctatctcagttggagatctccgggaacaacttctccggcgagattccggggagaatatgcgacctcggagaactcagggtcgtcgatctcagccgtaaccgtttctcaggatctcttccgtcttgcattaaccgattgaagaatctggagagagtggagatgcaagagaacatgctcgacggcgagatcccgagttcactgagttcgtgcgccaagttaaccgagttgaaactctccgataaccgtcttcgcggcggaattccaccggagctcggggaattaccggttttgaattatctggatctctctagcaaccaactctccggtgagattccgccggagttgttgaagctgaagctgaatcagttcaacgtctccgataacagactctccggtaagatcccttctggttttcaacaagatatttttgtattgagtttcttgggtaacccgaaactctgtgccccggatatggatccgattcgaccttgccgatccaaacccgagcctcggttcgtcctggtgatctccgtcgtctgcatcgttgcactaatcggagctttggtttggctctttattaaaaccaaaccgttattccagagaaaaccgaaccggaccgataaagtaaccattttccagcgggtcgagttcactgaggaagacatatacccgcaattaacagaagacaacataatcgggtcgggtgggtcgggtttggtttatagagtgatactcaaatcaggcgaaacgctcgcggtgaagaaactttggggaggaccgggtcgtaaacccgaatccgaatctgttttcagatccgaggtagagattctgggtcgggttagacatggtaacatagtgaagcttctcatgtgttgcagcggcgaagagtttcgttttttagtgtacgagttcatggaaaacggcagcttgggtgacgttttgcattcggagaaagaacatcgtgccgtttctacgctggactggacgacacggttttcgatcgcccttggtgcagctcaaggacttgcttatctacatcatgactctgttccggcgattgttcaccgtgacgtcaaaagcaataatatattgttggaccatgagatgaagccacgtgttgctgatttcggtttagctaaaccgttgaaaagagaagacaataatagtgtctcacatgtttctcctatgtcttgtgttgctggatcctacggctacattgctccagaatatggttatacgatgagagtgaatgagaagagcgatgtgtatagttttggcgtcgtgttactcgagttgattactggtaaaagaccaaatgattcgtcgtttggggagaataaagacattgttaagtatgcaatggaatcagctttgtgttactcttctccatcacctggagacaaagctatgaatcaggattcacctggaaactttcgagatcttagtaagcttgtggatccaaagatggaactttctaggggggagtatgaagaggtagagaaagttcttgagattgcgttgctttgcacgtcgtcgtttccgattagtaggccgaccatgaggaaagttgtggagttgctcaaagagaagaagccacttgagtga

SEQ ID NO: 36 - protein, 988 aa, Brassica nigra; identical to SEQ ID NO: 27
  source 1..988: note = BniBHSL2 aa

SEQ ID NO: 37 - DNA, 2950 bp, Brassica carinata
  source 1..2950: mol_type = genomic DNA; note = BcaCHAE gene
atgctttattgtcttattctcctcctcctcctatgcctctcttccacgtatctatgtctctcactaaaccacgatgccaccatcctccggcaagccaaactgggcttatctgacccggcccagtcactatcttcctggtccgaaaacgacgtcactccgtgccactggcgcggcatcacctgcgatgccacgtcagctgtcttctccgttaatctctccagcttcatgctcgtcggtcctttcccttccatcctctgccgtcttccttctctgtcattcctctctctctataacaactccatcaacggttcctctccggcgatgattttgcatcgtgtcggaatctcatccatctcaatttgtcggagaatctcttggttggttccatcccggagtcactccctttcaagctgccgaacctccgccgcttcggaatatccgggaacaacttatcggacacgattccggcgagcttcggagagttccggaagctcgagaagttaaaccttgccggaaacctcctctccggcaccattcccgcctcgctcggcaacgtatccactctgagagaactcagactcgcttacaatatattttctcctggtcgaatcccgagtcaactcggtaatctaacggagcttcgtgttctctggctcgccggctgcaacctcgctggttcggtgcctcaaactctgtcccggttgactcacttggtcagcttggatttgaccatgaatcaactcaccgggtctgtcccgagttggatcacggagctgagaagcatcgagcaaatcgagatattcaacaactcgttctcaggcgagttaccggaggcgatgggtaacatgacgatgctaaagagtttcgacgcgtcgacgaacaagctgacggggaagatccccgacggcttggctcggttaaacctcgagtcgctcaacctcttcgagaacatgctcgaagggcccttgccggagagcataactcgctccaaaacgttaacggaactaaaactgttcaacaacaaactcaccggggagataccgagtcaactcggcgcgaactctccgttacagtacgtggacttttcgtataatcagttttccggcgagataccggcgaatatatgcggaggagggaagctagagtttttcatgcttatagggaactctttttccggtgaaataccgacaaatcttgaaagtgcaggagcttgacacgtgtcaggctaaataacaacaagctttccggtcacgttccggaagagttctggggtttgcctcgtatgtcgctgctcgaactctcggagaactcgttcaccggaagaatctctgagtccatcgccggtgcaaagaatctatcaaacctgagaatctcaaagaatcagttctcaggttcgattcccggcgaaatcggatcgcttaacggactcatcgagatcgccggatatgaaaatagcttctccggcgaggttactagtactttggtgaagcttaagcaactgagcaggctcgatctcagctctaatcaactctccggcgagattccacgtggaatccgtggctggaagaatctgaacgagctcaatttagctaataaccatctctccggtgaagtccctagagagctcggagacttgtcggttcttaactacctcgatctctcgaacaatcaattttccggtgagattccaccggagctccagaatctgaagctgaacattataaacctatcgtataatcatctcaccgggagagtaccgcctctctacgctaacaaaatctacgcttctagctttgtcggcaaccatgacttgtgcgtagatgatcacgacagtcgttggcggaagatcgcacggtctaaaaacatcggttatgtctggattctcctatctattttcatactcgcctgtatggtttcgtcgttggggttgttatgtttatcgccaactgcaagaagatgagagcatccaagagcggtagattctctgcgtccaagtggagaagcttccacaagcttcatttcagcgagcatgagatcgttgattgccttgacgaacggaacgtgatcggttctggttcctccggtaaagtctacagagtggagcttagtggcggagaagtagtagtagctgtgaagaaactaaacaaaacggctaaaggggtgaagacgattcgttgaacagagacgttttcgctgcggaggtggaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttccaccgattgtccaccgtgacgtgaagtctagtaacatattgctagatgcagattatggcgccaaagtagccgactttggtattgccaaaatcggtcaaatgaccggtactaaatcgccagaagctatgtctggaatcgctggttcttgcggttacattgcaccaggtatgataacataaccaaataaaatgataaaaccatacaagttacataatgtttcaaatgatattggtgcgttaatagtttttctttctaatgtttctgcagaatacgtatatacactcagggtgaatgaaaagagtgatatctacagttttggtatcgtgcttttggagttggtaacggggaagcaaccaacagatctagaacttggagataaagatatggtgaagtgggtgtgcactacccttgaccaaagcggtttagaaacggtgattgatcccaaacttgatcttgggttcaaagaagagattagcaaagtcattcacattggtcttctctgtacgagtcctctccctctaaa

SEQ ID NO: 38 - DNA, 2745 bp, Brassica carinata
  source 1..2745: mol_type = other DNA; note = BcaCHAE cDNA
atgctttattgtcttattctcctcctcctcctatgcctctcttccacgtatctatgtctctcactaaaccacgatgccaccatcctccggcaagccaaactgggcttatctgacccggcccagtcactatcttcctggtccgaaaacgacgtcactccgtgccactggcgcggcatcacctgcgatgccacgtcagctgtcttctccgttaatctctccagcttcatgctcgtcggtcctttcccttccatcctctgccgtcttccttctctgtcattcctctctctctataacaactccatcaacggttcctctccggcgatgattttgcatcgtgtcggaatctcatccatctcaatttgtcggagaatctcttggttgctgccgaacctccgccgcttcggaatatccgggaacaacttatcggacacgattccggcgagcttcggagagttccggaagctcgagaagttaaaccttgccggaaacctcctctccggcaccattcccgcctcgctcggcaacgtatccactctgagagaactcagactcgcttacaatatattttctcctggtcgaatcccgagtcaactcggtaatctaacggagcttcgtgttctctggctcgccggctgcaacctcgctggttcggtgcctcaaactctgtcccggttgactcacttggtcagcttggatttgaccatgaatcaactcaccgggtctgtcccgagttggatcacggagctgagaagcatcgagcaaatcgagatattcaacaactcgttctcaggcgagttaccggaggcgatgggtaacatgacgatgctaaagagtttcgacgcgtcgacgaacaagctgacggggaagatccccgacggcttggctcggttaaacctcgagtcgctcaacctcttcgagaacatgctcgaagggcccttgccggagagcataactcgctccaaaacgttaacggaactaaaactgttcaacaacaaactcaccggggagataccgagtcaactcggcgcgaactctccgttacagagcttgacacgtgtcaggctaaataacaacaagctttccggtcacgttccggaagagttctggggtttgcctcgtatgtcgctgctcgaactctcggagaactcgttcaccggaagaatctctgagtccatcgccggtgcaaagaatctatcaaacctgagaatctcaaagaatcagttctcaggttcgattcccggcgaaatcggatcgcttaacggactcatcgagatcgccggatatgaaaatagcttctccggcgaggttactagtactttggtgaagcttaagcaactgagcaggctcgatctcagctctaatcaactctccggcgagattccacgtggaatccgtggctggaagaatctgaacgagctcaatttagctaataaccatctctccggtgaagtccctagagagctcggagacttgtcggttcttaactacctcgatctctcgaacaatcaattttccggtgagattccaccggagctccagaatctgaagctgaacattataaacctatcgtataatcatctcaccgggagagtaccgcctctctacgctaacaaaatctacgcttctagctttgtcggcaaccatgacttgtgcgtagatgatcacgacagtcgttggcggaagatcgcacggtctaaaaacatcggttatgtctggattctcctatctattttcatactcgcctgtatgaagatgagagcatccaagagcggtagattctctgcgtccaagtggagaagcttccacaagcttcatttcagcgagcatgagatcgttgattgccttgacgaacggaacgtgatcggttctggttcctccggtaaagtctacagagtggagcttagtggcggagaagtagtagtagctgtgaagaaactaaacaaaacggctaaaggggtggaaacgcttggaacgatcaggcataagagtattgtgcgcttgtggtgttgctgcagctccggggactgcaagttgttggtatacgagtacatgcctaatgggagcttagctgatgtgttacacagccactgcaaaggcggggtgttgttgggctggcctgaacggttaaggatcggtttagacgcagccgagggtctatcatacttacaccacgattgtgttccaccgattgtccaccgtgacgtgaagtctagtaacatattgctagatgcagattatggcgccaaagtagccgactttggtattgccaaaatcggtcaaatgaccggtactaaatcgccagaagctatgtctggaatcgctggttcttgcggttacattgcaccagaatacgtatatacactcagggtgaatgaaaagagtgatatctacagttttggtatcgtgcttttggagttggtaacggggaagcaaccaacagatctagaacttggagataaagatatggtgaagtgggtgtgcactacccttgaccaaagcggtttagaaacggtgattgatcccaaacttgatcttgggttcaaagaagagattagcaaagtcattcacattggtcttctctgtacgagtcctctccctctaaaccgaccttccatgagaaaagttgtgatcatgcttcaagaagtctctggtgctgtttctattagcggtccaaatgcgtccaaacgctctaagagcagtgggaaactctcgccttactatgtggaagacgtaaacagcgtttga

SEQ ID NO: 39 - protein, 914 aa, Brassica carinata
  source 1..914: note = BcaCHAE aa
MLYCLILLLLLCLSSTYLCLSLNHDATILRQAKLGLSDPAQSLSSWSENDVTPCHWRGITCDATSAVFSVNLSSFMLVGPFPSILCRLPSLSFLSLYNNSINGSSPAMILHRVGISSISICRRISWLLPNLRRFGISGNNLSDTIPASFGEFRKLEKLNLAGNLLSGTIPASLGNVSTLRELRLAYNIFSPGRIPSQLGNLTELRVLWLAGCNLAGSVPQTLSRLTHLVSLDLTMNQLTGSVPSWITELRSIEQIEIFNNSFSGELPEAMGNMTMLKSFDASTNKLTGKIPDGLARLNLESLNLFENMLEGPLPESITRSKTLTELKLFNNKLTGEIPSQLGANSPLQSLTRVRLNNNKLSGHVPEEFWGLPRMSLLELSENSFTGRISESIAGAKNLSNLRISKNQFSGSIPGEIGSLNGLIEIAGYENSFSGEVTSTLVKLKQLSRLDLSSNQLSGEIPRGIRGWKNLNELNLANNHLSGEVPRELGDLSVLNYLDLSNNQFSGEIPPELQNLKLNIINLSYNHLTGRVPPLYANKIYASSFVGNHDLCVDDHDSRWRKIARSKNIGYVWILLSIFILACMKMRASKSGRFSASKWRSFHKLHFSEHEIVDCLDERNVIGSGSSGKVYRVELSGGEVVVAVKKLNKTAKGVETLGTIRHKSIVRLWCCCSSGDCKLLVYEYMPNGSLADVLHSHCKGGVLLGWPERLRIGLDAAEGLSYLHHDCVPPIVHRDVKSSNILLDADYGAKVADFGIAKIGQMTGTKSPEAMSGIAGSCGYIAPEYVYTLRVNEKSDIYSFGIVLLELVTGKQPTDLELGDKDMVKWVCTTLDQSGLETVIDPKLDLGFKEEISKVIHIGLLCTSPLPLNRPSMRKVVIMLQEVSGAVSISGPNASKRSKSSGKLSPYYVEDVNSV

SEQ ID NO: 40 - DNA, 3895 bp, Brassica carinata
  source 1..3895: mol_type = genomic DNA; note = BcaBHSL2 gene
atgaccaccattactacttctctcttcttgtcacttgttctttcttgtgttctccaagtttcttccaatggagacgccgagatactgagccgagtcaaaaggagcaggctctctgacccggacggaaagttaaatgattggatcataaccggagataaccggaatccctgtaactggacgggaattaaatgcgactccaaaaaccgttccgtcacggcaattgatctctccggctacggactctccggtgggttcccttacgagttctgccgtgtacgtaccctcgtcaacatcactctctccgaaaacaacctcaacggtacgatagattcagctcctctctccctatgttctcgaatccacgtcctgatcctcaacgaaaacggcttctccgggaagttaccggaattttcgccggagtttcgtagcctacgcgtcatcgaactggaatccaacttgttcaccggcgagattcccgaaagctacggaaagcttacatctctccgagttctgaatctcaacggcaactcgctcggcggaatcgttccggcgtttttgggtaacctgaccgagctgactcgtctcgagctcgcgtacgtccgattcgagcccggtccgattccgtcgacgttcgggagcttgacgaagctgagttacctccggctaactaactcgaacctcgtcggagagattcctgattcgatcgggaatctggtttcgatttagctacgaacggtctctccggagagataccggagagcatcggaaagctaaaatcggtttatcagttggagctttacgacaataatctatctggaaaactgccggagagcatcgggaacttaacggctatgaggaatttcgacgtttcgcagaataatctcaccggtgagttacctgagaagatcgcttctctccagctcgtttcatttaatctcaatgataacttcttcaacggtgagttgccacgtgtcgtggctttgaatcctaatctcgtcgagttcaagatcttcaacaacagcttcacagggacgttgcctaggagtttcggcaaattctcaggtgtaaccgagttcgacgtctcgacgaatagattctctggtgaattgccgccttatctctgctacatgaagaaactcgagaggctaatcagtttcggcaatcggattaaaggcgagattccggaatcttacggcgagtgtgactcgcttaattacattcgaacggcggataacaaactctccggcgaagttccggttaaggtttgggagcttcctcttactcgtctcgagctatctaacaatcaattacaaggttcgattcctccgtcgatatccaatgctcgtaatctatctcagctcgaaatctccgggaacaacttctccggcgagattccggggagaatctgcgacctcggagaactcagggtcgtcgatctcagccgtaaccgtttctccggatctcttccgtcttgcattaaccgattgaaaaatctggagagagtggagatgcaagagaacatgctcgacggcgagattccgagttcactgagttcgtgctccaagttaaccgagttgaaactctccgataaccgtctccgcggcggaattccaccgaagctcggggaattaccggttttgaattacctggatctttcgaacaaccaactctccggtgagattccgccggagttgttgaagctgaagctgaaccagttcgacgtctccgataacagactctccggtaagatcccttctggctttcaacaagatatttttctacggagtttctttggtaacccggatctctgtgccccggatatggatccgattcgaccttgccgatccaaacccgagcctcggttcgtcctggtgatctccgtcgtctgcatcgttgcactaatcggagctttggtttggctctttattaaaaccaaaccgttattccagagaaaaccgaagcggaccgataaagtaaccattttccagcgggtcgggttcactgaggaagacgtatacccgcaattaggcctgggcaaaataaccggaaccgaaaaaccgaaccggaaccgaaccgaaatacccgaaaccggaaccagactaatactctcaaatacccgagcggttcctatattttatatcgaaataaccgaaccgaaccgaacgaaccgaaccgaaccgaaccgaatgggtaccgaatatataaaaatattaattatatatacatataacatcactaaatatatatttttaatttaaaattctattaaaagtatcgaaaatagttgaggataactaaattattataaagtatccaaactaccgaaagtatcgaaactatcggatagttttatccgaaatatccaaagtaatcgaaatatccaaatttttatctaaatcatcctaattatttgatattttaccctaaataaccgatattttatccaaattatccgaactaccgaactatcgaaccgaaccggatccaaatgagaacgaacttttcggatattttcggttcctacatttactatcgaacgaaccgaaccgaaactatcgaaccgaaccgaaccgaaaataggtcaaatactaaatggatcctctagccctatccgaactacccaaaaccgaaatacccgaaccgaaccgaaccgatacccgaaatgcccaggcctacccgcaattaacagaagacaacataatcggttcgggcgggtcgggtttggtttacagagtgacactcaaatcaggcgaaacgctcgcggtgaagaaactttggggaggaccgggtcgtaaacccgaatccgaatctgttttcagatccgaggtagagattctgggtcgggttagacatggtaacatagtgaagcttctcatgtgttgcagcggcgaagagtttcgttttttagtgtacgagttcatggaaaacggcagcttgggtgacgttttgcattcggagaaagaacatcgtgccgtttctacgctggactggacgacacggttttcgatcgcccttggtgcagctcaaggacttgcttatctacatcatgactctgttccggcgattgttcaccgtgacgtcaaaagcaataatatattgttggaccatgagatgaagccacgtgttgctgatttcggtttagctaaaccgttgaaaagagaagacaataatagtgtctcacatgtttctcctatgtcttgtgttgctggatcctacggctacattgctccaggttcgaattcttaaccgaaataaaccgaaacactactagtatagaccagaaaaccaaatactatacttctattccacaaagataaacattttagaaagtgtgtttcacaaagattatatatttgatataataaaattaaagtttattgacttaatatttgatgtaatttactgaaaattgataaacatacatattttaatatacttgaataaatgaaaaataagtttttgtggaacagaaaacttaccgttgttttgttcttttgttgtgattgcacatgcagaatatggttatacgatgagagtgaatgagaagagcgatgtgtatagttttggcgtcgtgttactcgagttgattactggtaaaagaccaaatgattcgtcgtttggggagaataaagacattgttaagtatgcaatggaatcagctttgtgttactcttctccatcacctggagacaaagctatgaatcaggattcacctggaaactttcgagatcttagtaagcttgtggatccaaagatggaactttctaggggggagtatgaagaggtagagaaagttcttgagattgcgttgctttgcacgtcgtcgtttccgattagtaggccgaccatgaggaaagttgtggagttgctcaaagagaagaagccacttgagtga

SEQ ID NO: 41 - DNA, 2918 bp, Brassica carinata
  source 1..2918: mol_type = other DNA; note = BcaBHSL2 cDNA
atgaccaccattactacttctctcttcttgtcacttgttctttcttgtgttctccaagtttcttccaatggagacgccgagatactgagccgagtcaaaaggagcaggctctctgacccggacggaaagttaatgattggatcataaccggagataaccggaatccctgtaactggacgggaattaaatgcgactccaaaaaccgttccgtcacggcaattgatctctccggctacggactctccggtgggttcccttacgagttctgccgtgtacgtaccctcgtcaacatcactctctccgaaaacaacctcaacggtacgatagattcagctcctctctccctatgttctcgaatccacgtcctgatcctcaacgaaaacggcttctccgggaagttaccggaattttcgccggagtttcgtagcctacgcgtcatcgaactggaatccaacttgttcaccggcgagattcccgaaagctacggaaagcttacatctctccgagttctgaatctcaacggcaactcgctcggcggaatcgttccggcgtttttgggtaacctgaccgagctgactcgtctcgagctcgcgtacgtccgattcgagcccggtccgattccgtcgacgttcgggagcttgacgaagctgagttacctccggctaactaactcgaacctcgtcggagagattcctgattcgatcgggaatctgataccggagagcatcggaaagctaaaatcggtttatcagttggagctttacgacaataatctatctggaaaactgccggagagcatcgggaacttaacggctatgaggaatttcgacgtttcgcagaataatctcaccggtgagttacctgagaagatcgcttctctccagctcgtttcatttaatctcaatgataacttcttcaacggtgagttgccacgtgtcgtggctttgaatcctaatctcgtcgagttcaagatcttcaacaacagcttcacagggacgttgcctaggagtttcggcaaattctcaggtgtaaccgagttcgacgtctcgacgaatagattctctggtgaattgccgccttatctctgctacatgaagaaactcgagaggctaatcagtttcggcaatcggattaaaggcgagattccggaatcttacggcgagtgtgactcgcttaattacattcgaacggcggataacaaactctccggcgaagttccggttaaggtttgggagcttcctcttactcgtctcgagctatctaacaatcaattacaaggttcgattcctccgtcgatatccaatgctcgtaatctatctcagctcgaaatctccgggaacaacttctccggcgagattccggggagaatctgcgacctcggagaactcagggtcgtcgatctcagccgtaaccgtttctccggatctcttccgtcttgcattaaccgattgaaaaatctggagagagtggagatgcaagagaacatgctcgacggcgagattccgagttcactgagttcgtgctccaagttaaccgagttgaaactctccgataaccgtctccgcggcggaattccaccgaagctcggggaattaccggttttgaattacctggatctttcgaacaaccaactctccggtgagattccgccggagttgttgaagctgaagctgaaccagttcgacgtctccgataacagactctccggtaagatcccttctggctttcaacaagatatttttctacggagtttctttggtaacccggatctctgtgccccggatatggatccgattcgaccttgccgatccaaacccgagcctcggttcgtcctggtgatctccgtcgtctgcatcgttgcactaatcggagctttggtttggctctttattaaaaccaaaccgttattccagagaaaaccgaagcggaccgataaagtaaccattttccagcgggtcgggttcactgaggaagacgcctacccgcaattaacagaagacaacataatcggttcgggcgggtcgggtttggtttacagagtgacactcaaatcaggcgaaacgctcgcggtgaagaaactttggggaggaccgggtcgtaaacccgaatccgaatctgttttcagatccgaggtagagattctgggtcgggttagacatggtaacatagtgaagcttctcatgtgttgcagcggcgaagagtttcgttttttagtgtacgagttcatggaaaacggcagcttgggtgacgttttgcattcggagaaagaacatcgtgccgtttctacgctggactggacgacacggttttcgatcgcccttggtgcagctcaaggacttgcttatctacatcatgactctgttccggcgattgttcaccgtgacgtcaaaagcaataatatattgttggaccatgagatgaagccacgtgttgctgatttcggtttagctaaaccgttgaaaagagaagacaataatagtgtctcacatgtttctcctatgtcttgtgttgctggatcctacggctacattgctccagaatatggttatacgatgagagtgaatgagaagagcgatgtgtatagttttggcgtcgtgttactcgagttgattactggtaaaagaccaaatgattcgtcgtttggggagaataaagacattgttaagtatgcaatggaatcagctttgtgttactcttctccatcacctggagacaaagctatgaatcaggattcacctggaaactttcgagatcttagtaagcttgtggatccaaagatggaactttctaggggggagtatgaagaggtagagaaagttcttgagattgcgttgctttgcacgtcgtcgtttccgattagtaggccgaccatgaggaaagttgtggagttgctcaaagagaagaagccacttgagtga

SEQ ID NO: 42 - protein, 972 aa, Brassica carinata
  source 1..972: note = BcaBHSL2 aa
MTTITTSLFLSLVLSCVLQVSSNGDAEILSRVKRSRLSDPDGKLNDWIITGDNRNPCNWTGIKCDSKNRSVTAIDLSGYGLSGGFPYEFCRVRTLVNITLSENNLNGTIDSAPLSLCSRIHVLILNENGFSGKLPEFSPEFRSLRVIELESNLFTGEIPESYGKLTSLRVLNLNGNSLGGIVPAFLGNLTELTRLELAYVRFEPGPIPSTFGSLTKLSYLRLTNSNLVGEIPDSIGNLIPESIGKLKSVYQLELYDNNLSGKLPESIGNLTAMRNFDVSQNNLTGELPEKIASLQLVSFNLNDNFFNGELPRVVALNPNLVEFKIFNNSFTGTLPRSFGKFSGVTEFDVSTNRFSGELPPYLCYMKKLERLISFGNRIKGEIPESYGECDSLNYIRTADNKLSGEVPVKVWELPLTRLELSNNQLQGSIPPSISNARNLSQLEISGNNFSGEIPGRICDLGELRVVDLSRNRFSGSLPSCINRLKNLERVEMQENMLDGEIPSSLSSCSKLTELKLSDNRLRGGIPPKLGELPVLNYLDLSNNQLSGEIPPELLKLKLNQFDVSDNRLSGKIPSGFQQDIFLRSFFGNPDLCAPDMDPIRPCRSKPEPRFVLVISVVCIVALIGALVWLFIKTKPLFQRKPKRTDKVTIFQRVGFTEEDAYPQLTEDNIIGSGGSGLVYRVTLKSGETLAVKKLWGGPGRKPESESVFRSEVEILGRVRHGNIVKLLMCCSGEEFRFLVYEFMENGSLGDVLHSEKEHRAVSTLDWTTRFSIALGAAQGLAYLHHDSVPAIVHRDVKSNNILLDHEMKPRVADFGLAKPLKREDNNSVSHVSPMSCVAGSYGYIAPEYGYTMRVNEKSDVYSFGVVLLELITGKRPNDSSFGENKDIVKYAMESALCYSSPSPGDKAMNQDSPGNFRDLSKLVDPKMELSRGEYEEVEKVLEIALLCTSSFPISRPTMRKVVELLKEKKPLE

SEQ ID NO: 43 - DNA, 23 bp, synthetic construct
  source 1..23: mol_type = other DNA; note = gRNA1_ pGGG-Oslo 1
aagagaggcataggaggaggagg

SEQ ID NO: 44 - DNA, 23 bp, synthetic construct
  source 1..23: mol_type = other DNA; note = gRNA2_ pGGG-Oslo 1
agcccagtttggcttgccggagg

SEQ ID NO: 45 - DNA, 23 bp, synthetic construct
  source 1..23: mol_type = other DNA; note = gRNA3_ pGGG-Oslo 2
ggaccaggaagatagtgactggg

SEQ ID NO: 46 - DNA, 23 bp, synthetic construct
  source 1..23: mol_type = other DNA; note = gRNA4_ pGGG-Oslo 2
gtcactccgtgccactggcgcgg

SEQ ID NO: 47 - DNA, 23 bp, synthetic construct
  source 1..23: mol_type = other DNA; note = gRNA1_ pGGG-Oslo 2
gggtcataaccggagataaccgg

SEQ ID NO: 48 - DNA, 23 bp, synthetic construct
  source 1..23: mol_type = other DNA; note = gRNA2_ pGGG-Oslo 2
gtgattccagtccagttacaggg

SEQ ID NO: 49 - DNA, 23 bp, synthetic construct
  source 1..23: mol_type = other DNA; note = gRNA3_ pGGG-Oslo 1
gcaatagatctctccgactacgg

SEQ ID NO: 50 - DNA, 23 bp, synthetic construct
  source 1..23: mol_type = other DNA
tctccggtgggttcccgtacggg

(19) United States
(12) Patent Application Publication
(10) Pub. No.: US 2026/0265770 A1
(43) Pub. Date: Sep. 10, 2026

(54) BRASSICA PLANTS WITH IMPROVED SEED RETENTION

(71) Applicant: UNIVERSITETET I OSLO, Oslo (NO)

(72) Inventors: Reidunn AALEN, Oslo (NO); Melinka BUTENKO, Bekkestua (NO)

(73) Assignee: UNIVERSITETET I OSLO, Oslo (NO)

(21) Appl. No.: 19/160,918

(22) PCT Filed: Mar. 1, 2024
(86) PCT No.: PCT/EP2024/055439

§ 371 Date: Aug. 29, 2025

(30) Foreign Application Priority Data
Mar. 2, 2023 (GB) 2303077.8

(51) Int. Cl. C12N 15/82; C12N 9/22; C12N 15/11

(52) U.S. Cl. CPC C12N 15/8267; C12N 9/222; C12N 15/111; C12N 15/8201; C12N 2310/20

(57) ABSTRACT

The present invention relates to Brassica oilseed plants with improved agronomic traits, such as seed retention. More particularly, the invention provides a mutant or modified Brassica oilseed plant or part thereof comprising a heritable mutation in a HAESA (HAE) gene and in a HAESA-LIKE2 (HSL2) gene, wherein the mutant or modified plant is deficient in seed abscission in comparison to a corresponding unmutated or unmodified plant.

### FIELD OF THE INVENTION

The present invention relates generally to the field of Brassica plants, particularly Brassica oilseed plants with improved agronomic traits, such as seed retention. More particularly, the invention provides mutant or modified Brassica oilseed plants having one or more heritable mutations in genes that encode proteins involved in the control of cell separation during abscission (e.g. abscission of floral organs). The invention further provides seeds obtained from the mutant or modified Brassica oilseed plants, methods of producing the plants and seeds, and uses of the plants and seeds, e.g. to obtain oil (e.g. edible oil), oil products and by-products of the extraction and processing procedure.

### BACKGROUND TO THE INVENTION

Brassica oilseed plants represent some of the most economically important crops worldwide. In particular, oilseed rape (OSR; Brassica napus L.) is key source of plant-derived oils and plays a crucial role in the food and animal feed industries. OSR is the third largest source of plant-derived oil in the world, with a global production of nearly 27.3 million metric tons annually. The oil derived from OSR is highly valued because of its nutritional composition and is considered one of the healthiest vegetable oils for human consumption. In the EU about 20 million metric tons of oilseed is produced each year and is currently valued at about 370 € per ton. However, due to pre-harvest shedding and harvesting methods it is estimated that about 15-25% of the seed is lost. Thus, even a small reduction in the amount of seed lost each year will have a significant economic impact.

In Brassica oilseed plants the seeds develop inside a pod which has valves that open via a process called dehiscence when the seeds are mature. Mutant Brassica oilseed plants have been developed in which the dehiscence process has been delayed or seed pods are more resistant to shattering, but these are not optimal solutions because the seeds detach within the pod, meaning that harvest delays and robust harvesting methods still result in significant seed losses.

Accordingly, there remains a need for methods and systems for minimising seed loss (i.e. maximising seed yield) in Brassica oilseed plants.

### SUMMARY OF THE INVENTION

The shedding of leaves, flowers, floral organs, seeds and fruit, are natural events in the life of plants, assisting reproduction and securing adaptation to seasonal and other changing environmental conditions. Organs are abscised from the plant at specialized abscission zones (AZs) comprising specialised cells (AZ cells) where cell wall remodelling factors and degradation enzymes disassemble the pectineus middle lamella between adjacent AZ cell files, ultimately leading to cell separation. Floral organ abscission in Arabidopsis thaliana was established as a model system for organ shedding about 20 years ago. When pollination has taken place the sepals, petals and stamens have served their purpose. Thus, after pollination the AZ cells at the base of the organs to be shed gain competence to respond to regulatory signals that trigger expression of cell wall remodelling proteins, leading to a gradual loosening of cell wall rigidity followed by expansion of the AZ cells on the plant body side. Upon a final degradation of the matrix between adjacent AZ cell files, the organs detach and the AZ scar is covered by a protective layer.

In Arabidopsis the small secreted peptide INFLORESCENCE DEFICIENT in ABSCISSION (IDA) controls the final cell separation step by binding to, and activating, the AZ cell-expressed membrane-bound leucine-rich repeat (LRR) receptor kinases (RKs) HAESA (HAE) and HAE-LIKE2 (HSL2) and co receptors belonging to the SOMATIC EMBRYOGENESIS RECEPTOR KINASE (SERK) family. The genes encoding the HAE and HSL2 receptors are redundant in function, in that the single mutants, hae and hsl2, look like wild-type plants, while double mutant haehsl2 plants retain their floral organs indefinitely similar to ida-1 mutant plants.

Phylogenetic analyses have revealed conservation of IDA and HSL2 in all orders of flowering plants. Abscission-associated expression patterns of presumptive IDA and HSL orthologues have been observed in AZs of fruits or leaves of oil palm, litchi, yellow lupin, citrus, poplar, soybean and tomato. Furthermore, it has been demonstrated that overexpression of IDA orthologues can rescue the ida mutant phenotype in Arabidopsis, which lends support to the hypothesis that IDA-HAE/HSL2 signalling may control cell separation events in flowering plants.

However, prior to the present invention, it had not been demonstrated that the HAE/HSL2 receptors are necessary for organ abscission in species other than A. thaliana.

In work leading to the present invention, the inventors identified orthologues of the IDA/IDA-LIKE peptide family and the HAE and HSL2 receptors of B. rapa. B. rapa sheds floral organs like Arabidopsis, and is a close relative of the important crop B. napus (canola, oil seed rape). Interestingly and unexpectedly, analysis of the abscission phenotypes of B. rapa haehsl2 double mutants revealed that the double mutants are not only deficient in floral organ abscission, like their Arabidopsis counterparts, but also fail to abscise their seeds. This surprising finding led the inventors to propose a new solution to the problem of seed loss in Brassica oilseed plants, namely reduction in pre-harvest seed detachment in Brassica oil seed crops by the mutation or modification of genes encoding the receptors HAE and HSL2.

Accordingly, in one aspect the invention provides a mutant or modified Brassica oilseed plant or part thereof comprising a heritable mutation in a HAE gene and in a HSL2 gene, wherein the mutant or modified plant is deficient in seed abscission in comparison to a corresponding unmutated or unmodified plant.

In another aspect the invention provides seed of the mutant or modified Brassica oilseed plant disclosed herein, wherein the seed carries the heritable mutations disclosed herein.

In a further aspect, the invention provides a method for producing a mutant or modified Brassica oilseed plant that is deficient in seed abscission in comparison to a corresponding unmutated or unmodified plant, the method comprising modifying a Brassica oilseed plant or cell to decrease the expression of a HAE gene and an HSL2 gene and/or to decrease the activity of the proteins encoded by said genes in comparison to the expression and/or activity in a corresponding unmutated or unmodified plant. It will be understood that when the method involves modifying a Brassica oilseed plant cell, it may further comprise a step of generating a plant from said cell comprising the mutation/modification.

In yet another aspect the invention provides a modified Brassica oilseed plant obtainable from the method disclosed herein, wherein the plant comprises a genetic modification (e.g. transgene or edited gene) that results in decreased expression of one or more (preferably all) HAE genes and one or more (preferably all) HSL2 genes and/or decreased activity of the proteins encoded by said genes in comparison to the expression and/or activity in a corresponding unmodified plant.

In still another embodiment the invention provides seed obtainable from the modified Brassica oilseed plant disclosed herein, wherein the seed comprises a genetic modification (e.g. transgene or edited gene) that results in decreased expression of one or more (preferably all) HAE genes and one or more (preferably all) HSL2 genes and/or decreased activity of the proteins encoded by said genes in a plant grown from the seed in comparison to the expression and/or activity in a corresponding plant grown from unmodified seed.

In a further aspect, the invention provides the use of:
(i) a plant or part thereof as disclosed herein;
(ii) seed as disclosed herein; or
(iii) a plant produced by the method disclosed herein to:
(a) breed a plant;
(b) grow a crop; or
(c) produce seed.

In another aspect, the invention provides the use of:
(i) a plant or part thereof as disclosed herein;
(ii) seed as disclosed herein; or
(iii) a plant produced by the method disclosed herein,
to obtain oil (e.g. edible oil) and/or one or more by-products from the oil extraction and/or processing procedure.

In yet another aspect, the invention provides a method of obtaining the seed as disclosed herein comprising:
(a) providing:
(i) a plant or part thereof (e.g. seed pod) as disclosed herein; or
(ii) a plant produced by the method disclosed herein; and
(b) harvesting and/or separating seed from the plant or part thereof of (a).

In a still further aspect, the invention provides a method of obtaining oil comprising:
(a) providing seed as disclosed herein (e.g. seed obtained from the method disclosed herein); and
(b) extracting oil from the seed of (a).

### DETAILED DESCRIPTION

A “HAESA (HAE) gene” as used herein refers to a gene which encodes a cell-expressed membrane-bound leucine-rich repeat (LRR) receptor kinase (RK) that functions as a receptor for an IDA (INFLORESCENCE DEFICIENT IN ABSCISSION) peptide ligand. In particular, a HAE gene refers to a gene that encodes an LRR-RK that functions in the signalling module that controls the separation (detachment) of one or more floral organs. The IDA-HAE/HSL2 signalling module principally has been characterised in the model species Arabidopsis thaliana, whereas the present invention is concerned with Brassica oilseed plants. Thus, unless specified otherwise, the terms HAESA gene and HAE gene refer to an orthologue of the Arabidopsis HAESA gene (At4g28490). As shown herein, an orthologue of the Arabidopsis HAESA gene according to the invention functions in the signalling module that controls seed detachment. Accordingly, a HAE gene refers to a gene that encodes an LRR-RK that functions as a receptor for an IDA peptide ligand encoded by an orthologue of the Arabidopsis IDA gene. In particular, the HAE LRR-RK functions as a receptor for an IDA peptide ligand encoded by an orthologue of the Arabidopsis IDA gene from the same species as the HAE LRR-RK. As some species may contain more than one orthologue of the Arabidopsis IDA gene, the HAE LRR-RK may function as a receptor for more than one IDA peptide ligand, each encoded by a separate orthologue of the Arabidopsis IDA gene. Moreover, due to homology between HAE and IDA genes from different species, the LRR-RK protein encoded by a HAE gene as disclosed herein may also function as a receptor for the IDA peptide ligand encoded by the Arabidopsis IDA gene.

As noted above, the receptors encoded by the HAE and HSL2 genes are redundant in function. Accordingly, a “HAE-LIKE2 (HSL2) gene” as used herein refers to a gene which encodes a cell-expressed membrane-bound LRR-RK that functions as a receptor for an IDA peptide ligand. In particular, a HSL2 gene refers to a gene that encodes an LRR-RK that functions in the signalling module that controls the separation (detachment) of one or more floral organs. Unless specified otherwise, the terms HAE-LIKE2 gene and HSL2 gene refer to an orthologue of the Arabidopsis HAE-LIKE2 (HSL2) gene (At5g65710). As shown herein, an orthologue of the Arabidopsis HSL2 gene according to the invention functions in the signalling module that controls seed detachment. Accordingly, a HSL2 gene refers to a gene that encodes an LRR-RK that functions as a receptor for an IDA peptide ligand encoded by an orthologue of the Arabidopsis IDA gene. In particular, the HSL2 LRR-RK functions as a receptor for an IDA peptide ligand encoded by an orthologue of the Arabidopsis IDA gene from the same species as the HSL2 LRR-RK. As some species may contain more than one orthologue of the Arabidopsis IDA gene, the HSL2 LRR-RK may function as a receptor for more than one IDA peptide ligand, each encoded by a separate orthologue of the Arabidopsis IDA gene. Moreover, due to homology between HSL2 and IDA genes from different species, the LRR-RK protein encoded by an HSL2 gene as disclosed herein may also function as a receptor for the IDA peptide ligand encoded by the Arabidopsis IDA gene.

An “IDA gene” (also known as an “IDL gene”) as used herein refers to a gene which encodes a peptide ligand that functions as a ligand for an LRR-RK, particularly a HAE and/or HSL2 LRR-RK. In particular, an IDA gene refers to a gene that encodes a peptide ligand that functions in the signalling module that controls the separation (detachment) of one or more floral organs. Unless specified otherwise, the term IDA gene refers to an orthologue of the Arabidopsis IDA gene (At1g68765). Accordingly, an IDA gene refers to a gene that encodes a peptide ligand that functions as a ligand for a HAE and/or HSL2 receptor encoded by an orthologue of the Arabidopsis HAE and/or HSL2 genes. In particular, the IDA peptide functions as a ligand for a HAE and/or HSL2 receptor encoded by orthologues of the Arabidopsis HAE and/or HSL2 genes from the same species as the IDA peptide ligand. As some species may contain more than one orthologue of the Arabidopsis HAE and/or HSL2 genes, the IDA peptide ligand may function as a ligand for more than one HAE and/or HSL2 receptor, each encoded by a separate orthologue of the Arabidopsis HAE and/or HSL2 genes. Moreover, due to homology between HAE, HSL2 and IDA genes from different species, the IDA peptide ligand protein encoded by an IDA gene as disclosed herein may also function as a ligand for the HAE and HSL2 receptors encoded by the Arabidopsis HAE and HSL2 genes.

The term “functions as a receptor for an IDA peptide ligand” means that the LRR-RK participates in transducing the message of effective binding to IDA into the interior of a particular cell, e.g. a cell in an abscission zone (AZ), i.e. an AZ cell, via intracellular signalling domains to elicit a cell function, e.g. activation of cellular responses associated with IDA binding to the ectodomain of the LRR-RK. The intracellular signalling domains are comprised within the endodomain of the LRR-RK. The term “cell function” refers to a specialized function of a cell. Cell function of an AZ cell, for example, may involve activation of genes associated with organ abscission, e.g. genes encoding cell wall remodelling factors and degradation enzymes that function to disassemble the pectineus middle lamella between adjacent cell files. Thus, the term “functions as a receptor for an IDA peptide ligand” refers to the ability of the LRR-RK to transduce a function signal and that directs a cell to perform a specialized function. It will be appreciated that expression of the LRR-RK in some cells may not result in the induction of a cell function (e.g. because the IDA peptide ligand is not expressed). However, the LRR-RK, e.g. HAE or HSL2, should be capable of signalling once expressed within an appropriate cell type, e.g. an AZ cell.

The term “orthologue” or “ortholog” refers to genes in different species that evolved from a common ancestral gene by speciation. Thus, orthologues differ from paralogues, which are genes in the same organism that are a result gene duplication within a species. After duplication, paralogous genes/proteins experience weaker evolutionary pressure and their specificity diverges leading to new specificities and functions. In contrast, orthologous genes/proteins, typically are under similar regulation and therefore have the same or similar function and/or specificity, particularly in closely related organisms. Thus, orthologous proteins (i.e. encoded by orthologous genes) share at least one core function and may have additional functions. In the present case, the inventors have determined that HAE and HSL2 orthologues in Brassica oilseed plants have an additional function relative to the corresponding Arabidopsis genes, namely control of seed detachment or seed abscission. As noted above, there is functional redundancy between the HAE and HSL2 genes meaning that both genes must be disrupted (e.g. modified or mutated) to decrease their expression and/or the function of the encoded proteins to observe or achieve a phenotype associated with disruption of the IDA-HAE/HSL2 signalling module, e.g. reduced seed abscission.

As polyploid events (e.g. hybridization events) have occurred regularly during plant evolution, it will be understood by the skilled person that some species may have multiple orthologues of the HAE, HSL2 and/or IDA genes described herein. For instance, hybridization between the diploid Brassica rapa (with genome AA) and Brassica oleracea (with genome CC) resulted in allotetraploid Brassica napus (with AACC genome), which therefore usually has orthologues derived from both species. As orthologous genes/proteins share common functions, it is expected that there may be functional redundancy of the orthologues in species with multiple orthologues. Thus, in the context of the present invention, it may be necessary to disrupt the expression and/or function of each orthologous gene and/or protein within a species (i.e. all of the HAE genes/proteins and all of the HSL2 genes/proteins) in order to achieve the desired phenotype, e.g. a plant that is deficient in seed abscission. However, it is thought that disrupting the expression and/or function of at least one of each orthologous gene and/or protein within a species (i.e. at least one HAE gene/protein and at least one HSL2 gene/protein) may be sufficient to achieve the desired phenotype. For instance, this may result in partial disruption of the seed abscission control mechanism, thereby increasing seed retention relative to wild-type plants (i.e. unmutated or unmodified plants). In some instances, it may be advantageous to disrupt the expression and/or function of each of one orthologous gene and/or protein and at least one of the other orthologous genes and/or proteins within a species (i.e. all of the HAE genes/proteins and at least one HSL2 gene/protein). It will be understood that the invention contemplates the disruption (e.g. mutation or modification) of any combination of HAE genes and HSL2 genes insofar as it results in a plant that is deficient in seed abscission in comparison to a corresponding unmutated or unmodified plant (e.g. a wild-type plant).

Thus, the plant may comprise a heritable mutation in more than one HAE gene and/or more than one HSL2 gene. In a preferred aspect, the plant may comprise a heritable mutation in all of the HAE genes and/or all of the HSL2 genes.

As the invention provides Brassica oilseed plants that are deficient in seed abscission (e.g. in comparison to a corresponding unmutated or unmodified plant), e.g. plants with increased seed retention, the terms “HAE gene” and “HSL2 gene” as used herein refer to genes from a Brassica oilseed plant. Thus, the HAE and HSL2 genes may be from any of Brassica napus, Brassica rapa, Brassica nigra, Brassica cretica and Brassica juncea. In a preferred aspect, the HAE and HSL2 genes are from Brassica napus or Brassica rapa, particularly Brassica napus. The sequence identifiers for the HAE and HSL2 genes, cDNA, and amino acid sequences from the above-mentioned Brassica oilseed plants are listed in Table 1 below.

Accordingly, the Brassica oilseed plant may be selected from Brassica napus (genome AACC), Brassica rapa (AA), Brassica nigra (BB), Brassica cretica and Brassica juncea (AABB). In preferred embodiments, the Brassica oilseed plant may be selected from Brassica napus and Brassica rapa.

In accordance with standard notation, HAE and HSL2 genes from different species may be specified by the addition of a prefix abbreviation of the species name, e.g. Brassica rapa is abbreviated to Bra, Brassica oleracea is abbreviated to Bol, Brassica napus is abbreviated to Bna etc. Thus, HAE from Brassica rapa may be denoted as BraHAE. Moreover, for species comprising more than one orthologue, further notations may be included to differentiate between each orthologue. For instance, B. napus contains genomes derived from B. rapa and B. oleracea, which are termed genomes A and C respectively. Thus, the Brassica napus HAE gene derived from B. rapa may be denoted as BnaAHAE and the Brassica napus HSL2 gene derived from B. oleracea may be denoted as BnaCHSL2. Additionally or alternatively, the orthologues may be differentiated by reference to the chromosome on which there are encoded. For instance, the Brassica napus HSL2 gene derived from B. oleracea is encoded on chromosome 9 of B. napus and therefore may be denoted as BnaC09HSL2.

Whilst not wishing to be bound by theory, it is hypothesised that in plants with multiple orthologues of the HAE and HSL2 genes, e.g. B. napus, it may be sufficient to disrupt (e.g. mutate or modify) only one group of HAE and HSL2 genes to achieve the seed retention phenotype. Thus, in some embodiments where the Brassica oilseed plant is B. napus, only the HAE and HSL2 genes/proteins derived from B. rapa (i.e. the A genome) are disrupted (e.g. mutated or modified), i.e. BnaAHAE and BnaAHSL2. Alternatively viewed, when the Brassica oilseed plant is B. napus, at least BnaAHAE and BnaAHSL2 are disrupted (e.g. mutated or modified). Thus, in one embodiment, the invention provides a mutant or modified Brassica napus plant or part thereof comprising a heritable mutation in the BnaAHAE gene and in the BnaAHSL2 gene, wherein the mutant or modified plant is deficient in seed abscission in comparison to a corresponding unmutated or unmodified plant. However, it will be understood that in some embodiments all of the HAE and HSL2 genes are disrupted, i.e. the HAE C and HSL2 C genes are also disrupted. Thus, in one embodiment, the invention provides a mutant or modified Brassica napus plant or part thereof comprising a heritable mutation in the BnaAHAE gene, the BnaCHAE gene, the BnaCHSL2 gene and in the BnaAHSL2 gene, wherein the mutant or modified plant is deficient in seed abscission in comparison to a corresponding unmutated or unmodified plant.

TABLE 1
HAE and HSL2 sequences.

| | SEQ ID Nos (gene/coding region, |
|---|---|
| Gene name | cDNA, amino acid, respectively) |
| BnaAHAE | SEQ ID Nos. 1-3* |
| BnaCHAE | SEQ ID Nos. 4-6* |
| BnaAHSL2 | SEQ ID Nos. 7-9 |
| BnaCHSL2 | SEQ ID Nos. 10-12 |
| BraHAE | SEQ ID Nos. 13-15 |
| BraHSL2 | SEQ ID Nos. 16-18 |
| BjuAHAE | SEQ ID Nos. 19-21 |
| BjuBHAE | SEQ ID Nos. 22-24 |
| BjuAHSL2 | SEQ ID Nos. 25-27 |
| BjuBHSL2 | SEQ ID Nos. 28-30 |
| BniBHAE | SEQ ID Nos. 31-33 |
| BniBHSL2 | SEQ ID Nos. 34-36 |

*SEQ ID NOs: 1 and 4 are full gene sequences also comprising a promoter sequence

The structures of the HAE and HSL2 genes in the Brassica genus are expected to be similar in all species. In a representative example, the BnaAHAE gene (SEQ ID NO: 1) comprises: a promoter sequence (nucleotides 1-1778 of SEQ ID NO: 1); a first exon (nucleotides 1779-4374 of SEQ ID NO: 1); an intron (nucleotides 4375-4459 of SEQ ID NO: 1); a second exon (nucleotides 4460-4860 of SEQ ID NO: 1); and a 3′UTR (nucleotides 4861-5034 of SEQ ID NO: 1). Thus, the coding region of the BnaAHAE gene comprises nucleotides 1779-4861 of SEQ ID NO: 1 or nucleotides 1779-5034 of SEQ ID NO: 1 including the 3′UTR. Similarly, the BnaCHAE gene (SEQ ID NO: 4) comprises: a promoter sequence (nucleotides 1-2019 of SEQ ID NO: 4); a first exon (nucleotides 2020-4612 of SEQ ID NO: 4); an intron (nucleotides 4613-4715 of SEQ ID NO: 4); a second exon (nucleotides 4716-5116 of SEQ ID NO: 4); and a 3′UTR (nucleotides 5117-5292 of SEQ ID NO: 4). Thus, the coding region of the BnaCHAE gene comprises nucleotides 2020-5116 of SEQ ID NO: 4 or nucleotides 2020-5292 of SEQ ID NO: 4 including the 3′UTR.

The BnaAHSL2 coding region (SEQ ID NO: 7) comprises: a first exon (nucleotides 1-2590 of SEQ ID NO: 7); an intron (nucleotides 2591-2684 of SEQ ID NO: 7); a second exon (nucleotides 2685-3065 of SEQ ID NO: 7); and a 3′UTR (nucleotides 3066-3198 of SEQ ID NO: 7). Similarly, the BnaCHSL2 coding region (SEQ ID NO: 10) comprises: a first exon (nucleotides 1-2584 of SEQ ID NO: 10); an intron (nucleotides 2585-2675 of SEQ ID NO: 10); a second exon (nucleotides 2676-3049 of SEQ ID NO: 10); and a 3′UTR (nucleotides 3050-3176 of SEQ ID NO: 10). Thus, the BnaAHSL2 coding region (SEQ ID NO: 7) comprises nucleotides 1-3065 of SEQ ID NO: 7 or nucleotides 1-3198 of SEQ ID NO: 7 including the 3′UTR. Similarly, the BnaCHSL2 coding region (SEQ ID NO: 10) comprises nucleotides 1-3049 of SEQ ID NO: 10 or nucleotides 1-3176 of SEQ ID NO: 10 including the 3′UTR.

It will be evident that where only the coding region is provided, the gene comprises an upstream (5′) region comprising a promoter sequence and a 5′UTR. Thus, the term “gene” as used herein refers to the genomic sequence comprising the regulatory sequences (e.g. promoter) and the transcribed sequence (i.e. the coding region). The terms “coding region” and “transcribed sequence” are used interchangeably herein and typically refer to the genomic sequence comprising the exons and introns and optionally the 5′UTR and 3′UTR. The coding region without the 5′UTR and 3′UTR sequences may be viewed as the “core” transcribed sequence. The terms “cDNA” and “coding sequence” refer to the transcribed sequence following the excision of introns.

Thus, the HAE gene as defined herein may encode a HAE polypeptide comprising an amino acid sequence selected from any one of SEQ ID NOs: 3, 6, 15, 21, 24, or 33. It is well established that plants (e.g. varieties) within a species may contain sequence variations, i.e. natural variations. Accordingly, the HAE gene as defined herein may encode a HAE polypeptide comprising an amino acid sequence with at least about 90% (e.g. at least about 95%, 96%, 97%, 98% or 99%) sequence identity to an amino acid sequence selected from any one of SEQ ID NOs: 3, 6, 15, 21, 24, or 33.

Similarly, the HSL2 gene as defined herein may encode a HSL2 polypeptide comprising an amino acid sequence selected from any one of SEQ ID NOs: 9, 12, 18, 27, 30, or 36 or a HSL2 polypeptide comprising an amino acid sequence with at least about 90% (e.g. at least about 95%, 96%, 97%, 98% or 99%) sequence identity to an amino acid sequence selected from any one of SEQ ID NOs: 9, 12, 18, 27, 30, or 36.

Thus, in a representative example, the invention provides a mutant or modified Brassica napus plant or part thereof comprising a heritable mutation in a HAE gene and in a HSL2 gene, wherein the mutant or modified plant is deficient in seed abscission in comparison to a corresponding unmutated or unmodified plant and wherein the unmodified or unmutated HAE gene encodes a HAE polypeptide comprising an amino acid sequence of SEQ ID NO: 3 or a HAE polypeptide comprising an amino acid sequence with at least about 90% (e.g. at least about 95%, 96%, 97%, 98% or 99%) sequence identity to an amino acid sequence of SEQ ID NO: 3 and the unmodified or unmutated HSL2 gene encodes a HSL2 polypeptide comprising an amino acid sequence SEQ ID NO: 9 or a HSL2 polypeptide comprising an amino acid sequence with at least about 90% (e.g. at least about 95%, 96%, 97%, 98% or 99%) sequence identity to an amino acid sequence of SEQ ID NO: 9.

In a further representative embodiment, the invention provides a mutant or modified Brassica napus plant or part thereof comprising a heritable mutation in both HAE genes and in both HSL2 genes, wherein the mutant or modified plant is deficient in seed abscission in comparison to a corresponding unmutated or unmodified plant and wherein the unmodified or unmutated HAE genes encode HAE polypeptides comprising amino acid sequences of SEQ ID NOs: 3 and 6 or amino acid sequences with at least about 90% (e.g. at least about 95%, 96%, 97%, 98% or 99%) sequence identity to amino acid sequences of SEQ ID NOs: 3 and 6 and the unmodified or unmutated HSL2 genes encode HSL2 polypeptides comprising amino acid sequences of SEQ ID NOs: 9 and 12 or amino acid sequences with at least about 90% (e.g. at least about 95%, 96%, 97%, 98% or 99%) sequence identity to amino acid sequences of SEQ ID NO: 9 and 12.

In some embodiments, the HAE gene as defined herein may comprise a nucleotide sequence selected from any one of SEQ ID NOs: 1, 4, 13, 19, 22, or 31 or a nucleotide sequence with at least about 90% (e.g. at least about 95%, 96%, 97%, 98% or 99%) sequence identity to a nucleotide sequence selected from any one of SEQ ID NOs: 1, 4, 13, 19, 22, or 31.

Similarly, the HSL2 gene as defined herein may comprise a nucleotide sequence selected from any one of SEQ ID NOs: 7, 10, 16, 25, 28, or 34 or a nucleotide sequence with at least about 90% (e.g. at least about 95%, 96%, 97%, 98% or 99%) sequence identity to a nucleotide sequence selected from any one of SEQ ID NOs: 7, 10, 16, 25, 28, or 34.

It will be understood that the natural nucleotide sequence variants defined above (i.e. the unmodified or unmutated sequences) encode the corresponding amino acid sequences defined above.

Thus, in a representative example, the invention provides a mutant or modified Brassica napus plant or part thereof comprising a heritable mutation in a HAE gene and in a HSL2 gene, wherein the mutant or modified plant is deficient in seed abscission in comparison to a corresponding unmutated or unmodified plant and wherein the unmodified or unmutated HAE gene comprises a nucleotide sequence of SEQ ID NO: 1 (particularly nucleotides 1779-4861 of SEQ ID NO: 1) or nucleotide sequence with at least about 90% (e.g. at least about 95%, 96%, 97%, 98% or 99%) sequence identity to a nucleotide sequence of SEQ ID NO: 1 (particularly nucleotides 1779-4861 of SEQ ID NO: 1) and the unmodified or unmutated HSL2 gene comprises a nucleotide sequence SEQ ID NO: 7 (particularly nucleotides 1-3065 of SEQ ID NO: 7) or a nucleotide sequence with at least about 90% (e.g. at least about 95%, 96%, 97%, 98% or 99%) sequence identity to a nucleotide sequence of SEQ ID NO: 7 (particularly nucleotides 1-3065 of SEQ ID NO: 7).

In a further representative embodiment, the invention provides a mutant or modified Brassica napus plant or part thereof comprising a heritable mutation in both HAE genes and in both HSL2 genes, wherein the mutant or modified plant is deficient in seed abscission in comparison to a corresponding unmutated or unmodified plant and wherein the unmodified or unmutated HAE genes comprise nucleotide sequences of SEQ ID NOs: 1 and 4 (particularly nucleotides 1779-4861 of SEQ ID NO: 1 and nucleotides 2020-5116 of SEQ ID NO: 4) or nucleotide sequences with at least about 90% (e.g. at least about 95%, 96%, 97%, 98% or 99%) sequence identity to nucleotide sequences of SEQ ID NOs: 1 and 4 (particularly nucleotides 1779-4861 of SEQ ID NO: 1 and nucleotides 2020-5116 of SEQ ID NO: 4) and the unmodified or unmutated HSL2 genes comprise nucleotide sequences of SEQ ID NOs: 7 and 10 (particularly nucleotides 1-3065 of SEQ ID NO: 7 and nucleotides 1-3049 of SEQ ID NO: 10) or nucleotide sequences with at least about 90% (e.g. at least about 95%, 96%, 97%, 98% or 99%) sequence identity to nucleotide sequences of SEQ ID NOs: 7 and 10 (particularly nucleotides 1-3065 of SEQ ID NO: 7 and nucleotides 1-3049 of SEQ ID NO: 10).

It will be understood that equivalent statements corresponding to the other species and sequences set out in Table 1 form embodiments of the invention and are not set out explicitly for conciseness.

As used herein, the term “mutation” encompasses a natural genetic variant or an engineered variant which is transmissible from parent to offspring. Thus, a mutation may be viewed as a heritable mutation and the terms “mutation” and “heritable mutation” are used interchangeably herein. It is preferred that the mutation is an engineered variant introduced by modifying a plant as defined below, i.e. the plant may be a modified plant comprising a mutation. In particular, the term “mutation” refers to a variation in a nucleotide sequence, i.e. a variation in comparison to a wild-type or native nucleotide sequence. In the context of the present invention, the term mutation refers to a variation in a HAE or HSL2 gene sequence. In particular, the heritable mutations described herein result in decreased expression of the HAE gene(s) and the HSL2 gene(s) in comparison to the expression in a corresponding unmutated or unmodified plant or result in decreased activity of the proteins encoded by the HAE gene(s) and the HSL2 gene(s) in comparison to the activity in a corresponding unmutated or unmodified plant.

Thus, a mutation may be in any part of the gene that results in the decreased expression or activity mentioned above. The mutation may be in the regulatory region (e.g. promoter) or coding region (e.g. coding sequence). In particular, the mutation may be in the 5′UTR, an exon, an intron or the 3′UTR. In particular, the mutation may be in an exon, particularly in the first exon. However, the mutation may be in the second exon.

It will be appreciated that the plant may comprise more than one mutation (i.e. a plurality of mutations, e.g. 3, 4, 5 or more) in a gene. Moreover, it will be understood that the mutations may be in any of the regions of the gene specified above. For instance, the plant may comprise a heritable mutation in the promoter and in the first exon of a gene (e.g. a HAE gene). Any combination of mutations that result in decreased expression or activity mentioned above are contemplated herein.

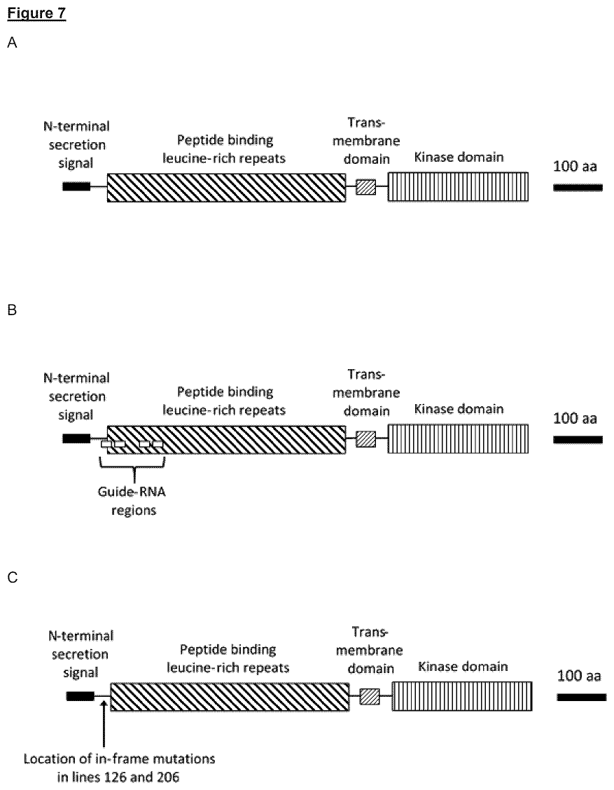

As discussed above, the proteins encoded by the HAE and HSL2 genes are membrane-bound leucine-rich repeat (LRR) receptor kinases (RKs). These LRR-RKs share a common structure comprising four principle domains or regions: 1) an N-terminal secretion signal domain, which functions to position the receptor in the cell membrane; 2) a leucine-rich repeat extracellular region which interacts with signalling peptides; 3) a transmembrane region, which anchors the receptor in the cell membrane; and 4) an intracellular kinase domain, which functions to initiate an intracellular signalling cascade when the LRR extracellular region interacts with its cognate signalling peptide(s). These domains are set out schematically in FIG. 7A. It will be appreciated that a mutation that disrupts (e.g. eliminates or abolishes) the function of any one of these domains or regions may facilitate the production of a mutant or modified plant as defined herein.

Thus, a mutation may be in any region or part of the HAE or HSL2 genes encoding one of the regions or domains of the receptor proteins as defined above. Accordingly, a mutation may be in a part of the gene encoding the N-terminal secretion signal domain, the leucine-rich repeat extracellular region, the transmembrane region or the intracellular kinase domain. As noted above, the plant may comprise a plurality of mutations and it will be understood that the mutations may be in any of the regions or parts of the gene encoding the regions or domains specified above. For instance, the plant may comprise a heritable mutation in a sequence encoding the N-terminal secretion signal domain and in a sequence encoding the leucine-rich repeat extracellular region. Alternatively, the plant may comprise a heritable mutation in a sequence encoding the intracellular kinase domain and in a sequence encoding the leucine-rich repeat extracellular region. In a further example, the plant may comprise a plurality of heritable mutations (e.g. 2, 3, 4 or more) in a sequence encoding a single region or domain, e.g. the leucine-rich repeat extracellular region or intracellular kinase domain, i.e. the plant may comprise a plurality of heritable mutations (e.g. 2, 3, 4 or more) in a sequence encoding the leucine-rich repeat extracellular region. A plant may comprise a plurality of heritable mutations (e.g. 2, 3, 4 or more) in a sequence encoding a plurality of regions or domain, e.g. a plurality of mutations in the leucine-rich repeat extracellular region and a plurality of mutations in the intracellular kinase domain. It will be understood that any combination of mutations that disrupt (e.g. eliminate or abolish) the function of the encoded protein or result in decreased activity of the encoded protein are contemplated herein.

As shown in the Examples, mutations may be targeted to any part of the genes described herein, particularly parts encoding a domain or region as defined above. Advantageously, a plant may contain one or more mutations in a part encoding a leucine-rich repeat extracellular region, particularly a part of the leucine-rich repeat extracellular region that is important for mediating the interaction with a cognate signalling peptide. For instance, the plant may contain one or more mutations in the part of the gene encoding the N-terminal portion of the leucine-rich repeat extracellular region.

A mutation can consist of an addition or deletion or substitution of one or more nucleotides in the gene which result in decreased expression or activity mentioned above. Addition or deletion mutations may involve the addition or deletion of one or more base pairs. Hence, 1, 2, 3, 4, 5, 6, 7, 8, 9 or 10 or more bases can be inserted or deleted. An addition mutation may involve insertion of an oligonucleotide or polynucleotide sequence, such as a T-DNA insertion. Similarly, a mutation may involve the deletion of a portion or part of a gene sequence. Thus, an addition or deletion may involve the insertion or deletion of a nucleotide sequence comprising 15, 20, 30, 40, 50, 100, 150, 200, 250 or more base pairs. In some preferred embodiments, the mutation is a deletion mutation, such as the deletion of 1-50, 1-20, 1-10, 1-5, e.g. 1, 2, 3 or 4 nucleotides/base pairs.

A mutation may be a substitution, which can occur at any position and may involve repetition (e.g. duplication) or inversion of fragments or segments of sequence.

A plurality of mutations may be a combination of substitution, addition and/or deletion mutations. Hence, a mutated gene can contain, for example, both substitution and deletion mutations. Further, a gene may contain more than one substitution and/or deletion mutation at different positions in the gene.

The number of mutations made may be in the range of 1 to 10, e.g. 2, 3, 4, 5, 6, 7, 8 or 9.

As noted above, it is preferred that the heritable mutation is effected (i.e. introduced or induced) by technical means, i.e. the heritable mutation is engineered and is not a naturally occurring mutation (i.e. variation). Any technical means may be used to induce the heritable mutation. Suitably, the heritable mutation may be effected by chemical mutagenesis, such as ethyl methanesulfonate (EMS) treatment, physical irradiation (e.g. UV mutagenesis, fast-neutron mutagenesis), insertional agents (e.g. including T-DNAs), or gene editing techniques (such as CRISPR/Cas). In some preferred embodiments, the mutation is induced or introduced using a gene editing technique, preferably a CRISPR/Cas gene editing technique.

The terms “modifying” or “modified” as used herein means a Brassica oilseed plant (e.g. a B. napus or B. rapa plant) that has been altered or changed at the genetic level. In this respect, it will be evident that the expression of a HAE gene and an HSL2 gene and the activity of their encoded proteins may be decreased indirectly, i.e. via genetic modifications (e.g. mutations) to other parts of the genome. For instance, nucleotide sequences that encode means for effecting RNA interference (RNAI), such as miRNA, siRNA, dsRNA or shRNA, may be inserted into a plant genome at a locus that is distinct from the locus of the HAE and/or HSL2 gene(s).

Thus, the present invention comprises the modification of plants using techniques for genetic modification of plants, which may be targeted (e.g. CRISPR/Cas) or non-targeted modifications (e.g. chemical mutagenesis followed by selection of mutations in the genes of interest, such as TILLING). It will be understood that mutant plants produced using chemical or irradiation mutagenesis techniques may be viewed as non-transgenic plants, insofar as the techniques do not involve the insertion of a transgene. Thus, the mutant or modified plants of the invention may be transgenic or non-transgenic plants. In some preferred embodiments, the mutant or modified plants are transgenic plants. Moreover, the non-transgenic mutant or modified plants (and related products, e.g. seeds etc.) contemplated herein are not exclusively obtained by means of an essentially biological process.

Thus, for instance, the invention provides a mutant or modified Brassica oilseed plant or part thereof comprising a heritable mutation in a HAE gene and in a HSL2 gene, wherein the mutant or modified plant is deficient in seed abscission in comparison to a corresponding unmutated or unmodified plant and wherein the plant is not exclusively obtained by means of an essentially biological process.

Accordingly, the invention also provides a transgenic Brassica oilseed plant or part thereof comprising a heritable mutation in a HAE gene and in a HSL2 gene, wherein the mutant or modified plant is deficient in seed abscission in comparison to a corresponding unmutated or unmodified plant.

The term “expression” of a gene (i.e. a HAE and/or HSL2 gene) or “gene expression” refers to the level of gene transcription (i.e. the level of mRNA encoded by the gene that is expressed/produced) and/or the level of translation, i.e. the level protein expression (of the protein encoded by the mRNA). Thus, gene expression may be decreased (or inhibited) by inhibiting transcription and/or translation. In particular, decreased expression refers to the level of transcription, i.e. the amount of mRNA produced. It will be understood that a decrease in the level of expression typically results in a decrease in translation, i.e. the level or amount of protein produced. Thus, decreased expression may refer a decrease in the level or amount of mRNA (encoded by the gene) and the level or amount of protein (encoded by the mRNA). It will be understood that a decrease in expression readily can be determined by measuring the level or amount of mRNA and/or protein in the mutant or modified plant (or a part thereof) and comparing the level(s) or amount(s) to the corresponding level(s) or amount(s) in a corresponding unmutated or unmodified plant (or part thereof), e.g. a wild-type plant. In particular, the level or amount of mRNA and/or protein may be determined in a cell (e.g. group of cells, such as abscission zone cells) or organ, e.g. flower, that normally expresses the gene(s), i.e. in the wild-type plant.

The “activity” of a protein encoded by a gene (i.e. a HAE and/or HSL2 gene) refers to its ability to function as described herein. In particular, the activity of a HAE and HSL2 protein refers to its ability to function as a receptor for an IDA peptide ligand as defined herein. It will be appreciated that protein activity in the plant or plant cells may be decreased (or inhibited) by inhibiting transcription and/or translation of the encoding gene. Alternatively or additionally, protein activity may be decreased by the introduction of a mutation that alters, abrogates or ablates the function of the encoded protein, e.g. a mutation that results in the substitution of a key amino acid in an active site or binding domain of the protein, or a mutation that results in a truncated protein. It will be understood that the decrease in protein activity can be determined using any suitable means known in the art, e.g. determining the presence and amount of a truncated protein. Conveniently, the activity may be determined by measuring the level or amount of seed abscission in the mutant or modified plant and comparing the levels or amounts to a corresponding unmutated or unmodified plant, e.g. a wild-type plant. Measuring the level or amount of seed abscission in a plant may be achieved using any suitable means, such as the shaking test described in the Examples below.

The term “abscission” refers to the shedding of a part of a plant, e.g. leaf, petal, sepal, stamen, fruit, seed etc. In Brassica oilseed plants (e.g. B. rapa), seeds are attached to the seed pod (silique) via a seed stick (funiculus) attached to the replum and detach (shed) prior to pod opening. However, as shown in the Examples, in mutant plants according to the invention (e.g. B. rapa haehsl2 double mutants and B. napus haehsl2 quadruple mutants) the seed stick and seed remains attached to the replum even after pod (silique) opening. Thus, the term “deficient in seed abscission” is used herein to mean that seeds in the mutant or modified plant do not detach (i.e. are not shed) from the seed pod. More particularly, seeds in the mutant or modified plant do not detach from the seed stick on the replum. In particular, the seeds in the mutant or modified plant do not detach (i.e. are not shed) from the seed pod prior to pod opening, e.g. the seeds in the mutant or modified plant do not detach from the seed stick on the replum prior to pod opening. Preferably, the seeds in the mutant or modified plant do not detach (i.e. are not shed) from the seed pod (e.g. replum) before or after pod opening. In this respect, it will be understood that detachment or shedding refers to the natural abscission process and does not include external means causing detachment, e.g. mechanical means, such as during seed/pod harvesting. In other words, it will be evident that the seeds in the mutant or modified plant may be detached or separated from the seed pod (e.g. replum) by the application of an external force and such external forces do not fall within the meaning of abscission, which is a natural process.

The deficiency in seed abscission does not necessarily mean that all seeds do not detach from (i.e. remain attached to) the seed pod (e.g. replum) as defined above. As used herein, the terms “seed pod” and “silique” refer to the whole pod including the seed sticks and the replum. The term “replum” refers to a thin separating wall or “dissepiment”, which separates the two valves of the seed pod (silique), and to which seeds are attached via a seed stick (funiculus). As discussed above, even a small reduction in seed loss during harvesting may have significant economic value. Accordingly, a mutant or modified plant may be viewed as being deficient in seed abscission if at least 10% of the total seeds remain attached to the seed pod (e.g. replum) after pod opening. Thus, in some embodiments, at least 15%, 20%, 30%, 40% or 50% of the total seeds remain attached to the seed pod (e.g. replum) after pod opening in the mutant or modified plant, e.g. up to 95%, 90%, 80%, 70% or 60% of the total seeds remain attached to the seed pod (e.g. replum) after pod opening in the mutant or modified plant. Conveniently, the deficiency in seed abscission may be quantified by comparing the proportion of seeds that remain attached to the seed pod (e.g. replum) after pod opening in the mutant or modified plant to the proportion of seeds that remain attached to the seed pod (e.g. replum) after pod opening in a corresponding unmutated or unmodified plant. A mutant or modified plant may be considered to be deficient in seed abscission when a greater proportion of seeds remain attached to the seed pod (e.g. replum) after pod opening in the mutant or modified plant in comparison to the corresponding unmutated or unmodified plant, e.g. the mutant or modified plant has at least 5%, 10%, 15%, 20%, 25%, 30%, 40%, 50% or more seeds attached to the seed pod (e.g. replum) after pod opening when compared to the corresponding unmutated or unmodified plant (e.g. a wild-type plant).

Thus, alternatively viewed, the mutant or modified plants have increased or improved seed retention (i.e. attachment to the seed pod (e.g. replum)), i.e. in comparison to a corresponding unmutated or unmodified plant. Suitably, a mutant or modified plant may be considered to have increased or improved seed retention when a greater proportion of seeds remain attached to the seed pod (e.g. replum) after pod opening in the mutant or modified plant in comparison to the corresponding unmutated or unmodified plant, e.g. the mutant or modified plant has at least 5%, 10%, 15%, 20%, 25%, 30%, 40%, 50% or more seeds attached to the seed pod (e.g. replum) after pod opening when compared to the corresponding unmutated or unmodified plant (e.g. a wild-type plant).

The number, proportion or amount of seeds that remain attached to the seed pod (e.g. replum) after pod opening may be measured by any convenient or suitable means. In particular, the average (mean) number, proportion or amount of seeds that remain attached to the seed pods (e.g. replums) after pod opening may be determined for a population of mutant or modified plants (e.g. a population of at least 5 plants, such as at least 10, 15 or 20 plants) and compared to the average (mean) number, proportion or amount of seeds that remain attached to the seed pods (e.g. replums) after pod opening in an equivalent population of corresponding unmutated or unmodified plants.

While the deficiency in seed abscission typically refers to the natural abscission process and does not include external means causing detachment, it may be convenient to determine or quantify the deficiency in seed abscission by assessing the level of seed retention following the application of an external force, e.g. a force sufficient to disrupt seed retention in wild-type plants. For example, the shaking test used in the Examples below represents a suitable test for assessing whether a mutant or modified plant is deficient in seed abscission, i.e. displays a seed retention phenotype, relative to an unmutated or unmodified plant. Thus, the percentages of seed retention mentioned above may apply to the shaking test, e.g. the average (mean) number, proportion or amount of seeds that remain attached to the seed pods (e.g. replums) after pod shaking as described in the Examples.

It is evident from the Examples that the mutant or modified plants may have multiple phenotypic characteristics that differ from unmutated or unmodified plants. For instance, insofar as some seeds detach from the replum in the seed pod in the mutant or modified plants, the detachment may be delayed in comparison to unmutated or unmodified plants. For instance, when comparing seed pods of the same developmental stage prior to pod opening (e.g. assessed based on the color of the seed pod) from mutant/modified plants to unmutated/unmodified, the number, proportion or amount of seeds that remain attached to the seed pods (e.g. replums) in the mutant/modified plants may be higher than the number, proportion or amount of seeds that remain attached to the seed pods (e.g. replums) in the unmutated/unmodified plants.

Furthermore, the mutant/modified plants may show reduced petal abscission, pod dehiscence and/or leaf (e.g. cauline leaf) abscission in comparison to unmutated/unmodified plants.

The effect of the mutations/modifications on pod dehiscence can be assessed using any suitable means. Conveniently, pod dehiscence can be measured using the shaking test described in the Examples. Thus, the mutant or modified plant may be deficient in seed abscission and pod dehiscence. Akin to the description of seed abscission above, a deficiency in pod dehiscence does not mean that seed pods do not open naturally. A mutant or modified plant may be viewed as being deficient in pod dehiscence if at least 5% or 10% of the total pods remain closed at maturity. Thus, in some embodiments, at least 15%, 20%, 30%, 40% or 50% of the total pods remain closed at maturity in the mutant or modified plant. Conveniently, the deficiency in pod dehiscence may be quantified by comparing the proportion of pods that remain closed at maturity in the mutant or modified plant to the proportion of pods that remain closed at maturity in a corresponding unmutated or unmodified plant. A mutant or modified plant may be considered to be deficient in pod dehiscence when a greater proportion of pods remain closed at maturity in the mutant or modified plant in comparison to the corresponding unmutated or unmodified plant, e.g. the mutant or modified plant has at least 5%, 10%, 15%, 20%, 25%, 30%, 40%, 50% or more pods that are closed at maturity when compared to the corresponding unmutated or unmodified plant (e.g. a wild-type plant).

Similarly to seed abscission, it may be convenient to determine or quantify the deficiency in pod dehiscence by assessing the level of pod opening following the application of an external force, e.g. a force sufficient to cause pod opening in wild-type plants. For example, the shaking test used in the Examples below represents a suitable test for assessing whether a mutant or modified plant is deficient in pod dehiscence, i.e. displays a reduced pod opening phenotype, relative to an unmutated or unmodified plant. Thus, the percentages of closed pods at maturity mentioned above may apply to the shaking test, e.g. the average (mean) number, proportion or amount of pods that remain closed after pod shaking as described in the Examples. For instance, at least 30% of seed pods from a mutant or modified plant may remain closed after 15 seconds of shaking, such as about 35%, 40%, 45%, 50% or more. Alternatively, the number of closed pods from a mutant or modified plant may be at least about 20%, 30%, 40%, or 50% higher than the number of closed pods from an unmutated or unmodified plant after 15 seconds of shaking, such as at least about 60%, 70%, 80% or more.

The terms “unmutated plant” and “unmodified plant” as defined herein refer to a plant (i.e. a Brassica oilseed plant) which has not been mutated or modified according to the present invention, e.g. to decrease the expression of a HAE gene and an HSL2 gene, and in which all other relevant features are the same (e.g. plant species, growing conditions, etc.). In one embodiment, an unmutated or unmodified plant is one which corresponds to the plant before it was mutated or modified. It will be appreciated therefore that the unmutated or unmodified plant may comprise mutations or modifications relative to a corresponding wild-type plant, but does not comprise mutation or modification in a HAE and HSL2 gene or that results in a decrease in the expression of a HAE and HSL2 gene or the activity of the encoded proteins. In a preferred embodiment, the unmutated or unmodified plant is a wild-type plant in which all other relevant features are the same (e.g. plant species, growing conditions, etc.).

As noted above, the heritable mutations or modifications may result in decreased expression of the HAE gene(s) and the HSL2 gene(s) in comparison to the expression in a corresponding unmutated or unmodified plant. Accordingly, the level of expression of the HAE and HSL2 genes in the mutant or modified plant may be about 80% or less of the expression in the corresponding unmutated or unmodified plant, e.g. about 70%, 60%, 50%, 40%, 30% or less. In some embodiments, the level of expression of the HAE and HSL2 genes in the mutant or modified plant may be less than about 20%, e.g. less than about 15%, 10% or 5% of the expression in the corresponding unmutated or unmodified plant.

Similarly, the heritable mutations or modifications may result in decreased activity of the proteins encoded by the HAE gene(s) and the HSL2 gene(s) in comparison to the activity in a corresponding unmutated or unmodified plant. Accordingly, the level of activity of the proteins encoded by the HAE and HSL2 genes in the mutant or modified plant may be about 80% or less of the activity in the corresponding unmutated or unmodified plant, e.g. about 70%, 60%, 50%, 40%, 30% or less. In some embodiments, the level of activity of the proteins encoded by the HAE and HSL2 genes in the mutant or modified plant may be less than about 20%, e.g. less than about 15%, 10% or 5% of the activity in the corresponding unmutated or unmodified plant. While reduced activity may be the result of reduced expression, and thus the mutant or modified plant may have reduced expression and activity, a reduction in activity can be achieved without a reduction in expression. Thus, the mutant or modified plant may express the mutated gene(s) at levels equivalent to the unmutated gene(s) in the corresponding unmutated or unmodified plant and still have decreased activity of the proteins encoded by the HAE gene(s) and the HSL2 gene(s) as defined above. Suitably, the mutant or modified plant may express the only the mutated gene(s). Thus, the mutant or modified plant may be homozygous for the mutation(s) and/or modification(s). Preferably, the mutant or modified plant may be homozygous for all of the mutations and/or modifications.

Thus, the mutant or modified plant may contain no endogenous (i.e. functional) HAE and HSL2 protein(s). Alternatively viewed, if any HAE and HSL2 proteins are present in the mutant or modified plant, it is preferred that they are in an inactive form. The inactive form may be a truncated form.

As noted above, the mutation or modification may interrupt or disrupt the nucleotide sequence that encodes a HAE or HSL2 protein as detailed herein. In particular, the mutation or modification may interrupt or disrupt an exon nucleotide sequence.

The interruption or disruption may cause the gene to not be transcribed and/or translated.

The nucleotide sequence may be interrupted or disrupted, for example, by deleting or otherwise modifying the ATG start codon of the nucleotide sequence such that translation of the protein is reduced or prevented.

The nucleotide sequence may comprise one or more nucleotide change(s) (i.e. variations, such as additions, deletions or substitutions) that reduce or prevent expression of the protein or affect protein trafficking. For example, expression of the protein may be reduced or prevented by introduction of a mutations that results in one or more premature stop codons, a frame-shift, a splice mutant or a non-tolerated amino acid substitution in the open reading frame.

A premature stop codon refers to a mutation which introduces a stop codon into the open reading frame and prevents translation of the entire amino acid sequence. The premature stop codon may be a TAG (“amber”). TAA (“ochre”), or TGA (“opal” or “umber”) codon.

A frame-shift mutation (also called a framing error or a reading frame shift) is a mutation caused by indels (insertions or deletions) of a number of nucleotides in a nucleotide sequence that is not divisible by three. Due to the triplet nature of gene expression by codons, the insertion or deletion can change the reading frame, resulting in a completely different translation product relative to the original coding sequence. A frameshift mutation will cause the reading of the codons after the mutation to code for different amino acids. The frameshift mutation will commonly result in the introduction of a premature stop codon.

A splice mutant inserts, deletes or substitutes a number of nucleotides in the specific site at which splicing takes place during the processing of precursor mRNA into mature mRNA. The deletion of the splicing site results in one or more introns remaining in the mature mRNA and may lead to the production of abnormal proteins.

A non-tolerated amino acid substitution refers to a mutation which causes a non-synonymous amino acid substitution in the protein which results in reduced or ablated function of the protein.

Any mutation that results in the expression of non-functional protein (e.g. an inactive and optionally truncated protein) or the absence of a functional protein, may be viewed as a knock-out mutation. Thus, any of the mutations or modifications described above may be a knock-out mutation.

Thus, a heritable mutation or modification as defined herein may be a frameshift mutation and/or result in a premature stop codon in the coding sequence relative to the corresponding unmutated gene. A heritable mutation or modification as defined herein may be splice mutant or a non-tolerated amino acid substitution in the open reading frame. In a preferred embodiment, a heritable mutation or modification as defined herein may be a knock-out mutation.

The term “part thereof” as used herein in the context of a Brassica oilseed plant (e.g. a B. napus or B. rapa plant) refers to a portion of the plant. For instance, a portion may refer to an organ, such as a seed, seed pod (i.e. fruit, silique), leaf, flower, stem, root etc. or a part of an organ, e.g. part of a seed pod, leaf etc. Preferred parts of the plants disclosed herein are seed pods (siliques) and seeds.

Accordingly, the invention may provide seed of the mutant or modified Brassica oilseed plant defined herein, wherein the seed carries the heritable mutations as defined herein.

As set out above, the invention also provides a method for producing a mutant or modified Brassica oilseed plant that is deficient in seed abscission in comparison to a corresponding unmutated or unmodified plant, the method comprising modifying a Brassica oilseed plant or cell to decrease the expression of a HAE gene and an HSL2 gene and/or to decrease the activity of the proteins encoded by said genes in comparison to the expression and/or activity in a corresponding unmutated or unmodified plant.

It will be understood from the discussion above that various methods of modifying plants are known in the art and any suitable method may be used to generate the mutant and modified plants disclosed herein.

For instance, the method may comprise introducing a heritable mutation in a HAE gene and in an HSL2 gene in said plant or cell, wherein the heritable mutations result in decreased expression of the HAE gene and the HSL2 gene and/or decreased activity of the proteins encoded by said genes in comparison to the expression and activity in a corresponding unmutated or unmodified plant.

Suitably, the method may comprise introducing a heritable mutation in all of the HAE genes and/or all of the HSL2 genes in said plant or cell, wherein the heritable mutations result in decreased expression of the HAE genes and the HSL2 genes and/or decreased activity of the proteins encoded by said genes in comparison to the expression and/or activity in a corresponding unmutated or unmodified plant.

Particular methods may comprise:
(i) genetically modifying a Brassica oilseed plant cell to: (1) introduce the heritable mutations defined herein; or (2) introduce a transgene that results in the decrease in expression and/or activity defined herein; and
(ii) regenerating a plant comprising the heritable mutations or transgene from the plant cell produced in (i).

As discussed in more detail below, the step of genetically modifying a Brassica oilseed plant cell may use CRISPR/Cas gene editing.

Moreover, the method may comprise a step of propagating the regenerated plant and obtaining seed comprising the heritable mutations or transgene.

Accordingly, a further aspect of the invention provides a modified Brassica oilseed plant obtainable from the method defined herein, wherein the plant comprises a genetic modification (e.g. transgene) that results in decreased expression of one or more (preferably all) HAE genes and one or more (preferably all) HSL2 genes and/or decreased activity of the proteins encoded by said genes in comparison to the expression and/or activity in a corresponding unmodified plant.

Moreover, the invention also provides seed obtainable from the modified Brassica oilseed plant defined above, wherein the seed comprises a genetic modification (e.g. transgene) that results in decreased expression of one or more (preferably all) HAE genes and one or more (preferably all) HSL2 genes and/or decreased activity of the proteins encoded by said genes in a plant grown from the seed in comparison to the expression and/or activity in a corresponding plant grown from unmodified seed.

Any method known in the art for providing a mutation or modification in a nucleic acid may be used in the present method. For example, homologous recombination may be used, in which a vector is created in which the relevant nucleotide sequences are mutated and used to transform plants or plant cells. Recombinant plants or plant cells expressing the mutated sequence may then be selected.

The nucleotide sequence may be wholly or partially deleted. The deletion may be continuous, or may comprise a plurality of sections of sequence. The deletion preferably removes a sufficient amount of nucleotide sequence such that the nucleic acid no longer encodes a functional protein. The deletion may, for example, remove 1, 2, 3, 4, 5% or more of the coding portion of the nucleotide sequence, such as at least 10, 20, 30, 40, 50, 60, 70, 80 or 90% of the coding portion of the nucleotide sequence.

The deletion may be total, in which case 100% of the coding portion of the nucleotide sequence is absent, when compared to the corresponding genome of a comparable unmutated or unmodified plant.

Methods for deletion of nucleotide sequences in plants are known in the art. For example, homologous recombination may be used, in which a vector is created in which the relevant nucleotide sequence(s) that are missing a portion are used to transform plants or plant cells. Recombinant plants or plant cells expressing the new portion of sequence may then be selected.

Suitably the method may comprise transforming a cell of a plant (e.g. a B. napus or B. rapa plant) with a genetic construct which is capable of inhibiting the expression of at least one HAE gene or at least one HSL2 gene (or a construct which is capable of inhibiting the expression of at least one HAE gene and at least one HSL2 gene in combination).

As noted above, nucleotide sequences that encode means for effecting RNA interference (RNAI), such as miRNA, SIRNA, dsRNA, shRNA or antisense RNA may be inserted into a plant genome at a locus that is distinct from the locus of the HAE and/or HSL2 gene(s). Thus, a “transgene” may be an RNAi construct or transgene, e.g. comprising a nucleotide sequence encoding a miRNA, siRNA, dsRNA, shRNA or antisense RNA, which results in a decrease in expression of the HAE and HSL2 genes defined herein. It will also be understood that a transgene may include a nucleotide sequence that is inserted in the HAE or HSL2 gene, which results in a decrease in their expression. Methods for introducing transgenes into plant cells are well-known in the art and any such method may be used in the method described herein.

Plant cells transformed with a transgene (e.g. via a vector) as described above may be grown and maintained in accordance with well-known tissue culturing methods, such as by culturing the cells (or tissue comprising the transformed cells, e.g. hypocotyl tissue) in a suitable culture medium supplied with the necessary growth factors such as amino acids, plant hormones, vitamins, etc. For instance, an efficient Agrobacterium-mediated transformation method using hypocotyl as explants for Brassica napus is disclosed in Dai et al., 2020 (Mol. Breeding, 40:96, incorporated herein by reference).

Modification of a nucleic acid sequence may be performed using targeted mutagenesis methods (also referred to as targeted nucleotide exchange (TNE) or oligo-directed mutagenesis (ODM)). Targeted mutagenesis methods include those employing zinc finger nucleases, TALENs (see WO2011/072246 and WO2010/079430), Cas9-like, Cas9/crRNA/tracrRNA or Cas9/gRNA CRISPR systems (see WO 2014/071006 and WO2014/093622), meganucleases (see WO2007/047859 and WO2009/059195), or targeted mutagenesis methods employing mutagenic oligonucleotides, possibly containing chemically modified nucleotides for enhancing mutagenesis with sequence complementarity to the gene, into plant protoplasts (e.g., KeyBase® or TALENs).

Alternatively, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate plant lines which comprise a gene encoding a protein having a mutation. TILLING uses traditional chemical mutagenesis (e.g. ethyl methanesulfonate (EMS) mutagenesis) followed by high-throughput screening for mutations. Thus, plants, seeds and tissues comprising a gene having the desired mutation(s) may be obtained.

The method may comprise the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such mutant or modified plants. Seeds may, for example, be irradiated or chemically treated and the plants may be screened for a modified phenotype, i.e. increased seed retention.

Mutant and modified plants may be distinguished from non-modified plants, i.e., wild type plants, by molecular methods, such as the mutation(s) present in the DNA, and by the modified phenotypic characteristics. The modified plants may be homozygous or heterozygous for the mutation, preferably homozygous.

Double-strand breaks (DSBs) can be induced by agents such as endonucleases that cleave the phosphodiester bond within a polynucleotide chain. These breaks can result in the induction of DNA repair mechanisms, including the non-homologous end-joining pathway, and homologous recombination. Endonucleases include a range of different enzymes, including Cas endonucleases, restriction endonucleases, meganucleases and TAL effector nucleases or TALENs. Methods of targeting DNA double-strand breaks have been described for TALENs, zinc finger nucleases and CRISPR-Cas endonucleases.

Any DSB-inducing agent may be used to introduce a heritable mutation or modification as described herein, e.g. may be used in the methods described herein. For instance, a DSB-inducing agent may be selected from a Cas endonuclease, recombinase, TALEN, zinc finger nuclease, restriction endonuclease or meganuclease. The DSB-inducing agent may be a Cas endonuclease. Alternatively viewed, the heritable mutation may be introduced following a DSB caused by a Cas endonuclease.

Thus, the heritable mutation or modification may be a targeted genomic modification or may be induced by a targeted genomic modification. In particular, the targeted genomic modification may comprise one or more (e.g. first and second) double strand breaks induced by a CRISPR-associated nuclease, e.g. Cas9. The introduction of one or more DSBs in the HAE and HSL2 genes may induce the non-homologous end-joining pathway that results in the introduction of heritable mutation or modification as described above. In particular, the heritable mutation or modification may be a deletion of one or more nucleotides (e.g. the excision of all or part of the gene, e.g. all or part of a coding sequence) that disrupts the expression and/or function of the gene and/or encoded protein as described herein.

As described above, it may be necessary to disrupt more than one HAE and HSL2 gene in some species, e.g. B. napus, in order to achieve or maximise the seed retention phenotype. As described in the Examples, the inventors have determined that it may be particularly advantageous to design guide RNAs based on regions of the genes that share high levels of sequence similarity or identity, such that a single guide RNA can target a CRISPR-associated nuclease, e.g. Cas9, to more than one HAE gene, e.g. both or all HAE genes, that are present. Similarly, a single guide RNA can target a CRISPR-associated nuclease, e.g. Cas9, to more than one HSL2 gene, e.g. both or all, HSL2 genes, that are present. It may be particularly advantageous to use more than one guide RNA for each type of gene (e.g. 2 or more), wherein each guide RNA can target a CRISPR-associated nuclease, e.g. Cas9, to a different region of the HAE or HSL2 genes. This approach may maximise the possibility of introducing DSBs and, as noted above, may result in the deletion or excision of all or part of a gene, e.g. all or part of a coding sequence.

In some embodiments, the heritable mutation or modification is induced by introduction of one or more DSBs in the HAE and HSL2 genes by a CRISPR-associated nuclease, e.g. Cas9. In particular, the CRISPR-associated nuclease, e.g. Cas9, may be targeted to the HAE and HSL2 genes using two or more guide RNAs for each gene, preferably wherein the guide RNAs are capable of targeting the CRISPR-associated nuclease, e.g. Cas9, to each copy (orthologue) of the HAE and HSL2 genes (e.g. the A and C copies the B. napus genes). In some preferred embodiments, the guide RNAs target the CRISPR-associated nuclease, e.g. Cas9, to regions in the coding sequence of the HAE and HSL2 genes (see e.g. FIG. 7B). In preferred embodiments, the guide RNAs target the CRISPR-associated nuclease, e.g. Cas9, to regions in the first exon of the HAE and HSL2 genes, such as regions within 300 or 250 nucleotides of the start codon of the genes. The guide RNAs may target the CRISPR-associated nuclease, e.g. Cas9, to regions within 200, 180, 160, 140, 120 or 100 nucleotides of the start codon of a HAE gene.

Thus, the guide RNAs may comprise a first guide RNA that targets the CRISPR-associated nuclease, e.g. Cas9, to a region within 50 nucleotides of the start codon of a HAE gene (e.g. to a region comprising nucleotides 25-35 of the coding sequence of HAE, e.g. nucleotides 1804-1814 of SEQ ID NO: 1 and nucleotides 2045-2055 of SEQ ID NO: 4) and a second guide RNA that targets the CRISPR-associated nuclease, e.g. Cas9, to a region within 100 nucleotides of the start codon of a HAE gene (e.g. to a region comprising nucleotides 90-100 of the coding sequence of HAE, e.g. nucleotides 1869-1879 of SEQ ID NO: 1 and nucleotides 2110-2120 of SEQ ID NO: 4). Thus, the guide RNAs may comprise a first guide RNA comprising a nucleotide sequence of SEQ ID NO: 43 and second guide RNA comprising a nucleotide sequence of SEQ ID NO: 44.

The guide RNAs may comprise a first guide RNA that targets the CRISPR-associated nuclease, e.g. Cas9, to a region within 140 nucleotides of the start codon of a HAE gene (e.g. to a region comprising nucleotides 125-135 of the coding sequence of HAE, e.g. nucleotides 1904-1914 of SEQ ID NO: 1 and nucleotides 2145-2155 of SEQ ID NO: 4) and a second guide RNA that targets the CRISPR-associated nuclease, e.g. Cas9, to a region within 170 nucleotides of the start codon of a HAE gene (e.g. to a region comprising nucleotides 155-165 of the coding sequence of HAE, e.g. nucleotides 1934-1944 of SEQ ID NO: 1 and nucleotides 2175-2185 of SEQ ID NO: 4). Thus, the guide RNAs may comprise a first guide RNA comprising a nucleotide sequence of SEQ ID NO: 45 and second guide RNA comprising a nucleotide sequence of SEQ ID NO: 46.

Similarly, the guide RNAs may comprise a first guide RNA that targets the CRISPR-associated nuclease, e.g. Cas9, to a region within 160 nucleotides of the start codon of a HSL2 gene (e.g. to a region comprising nucleotides 150-160 of the coding sequence of HSL2, e.g. nucleotides 150-160 of SEQ ID NO: 7 and SEQ ID NO: 10) and a second guide RNA that targets the CRISPR-associated nuclease, e.g. Cas9, to a region within 190 nucleotides of the start codon of a HSL2 gene (e.g. to a region comprising nucleotides 170-180 of the coding sequence of HSL2, e.g. nucleotides 170-180 of SEQ ID NO: 7 and SEQ ID NO: 10). Thus, the guide RNAs may comprise a first guide RNA comprising a nucleotide sequence of SEQ ID NO: 47 and second guide RNA comprising a nucleotide sequence of SEQ ID NO: 48.

The guide RNAs may comprise a first guide RNA that targets the CRISPR-associated nuclease, e.g. Cas9, to a region within 240 nucleotides of the start codon of a HSL2 gene (e.g. to a region comprising nucleotides 220-230 of the coding sequence of HSL2, e.g. nucleotides 220-230 of SEQ ID NO: 7 and SEQ ID NO: 10) and a second guide RNA that targets the CRISPR-associated nuclease, e.g. Cas9, to a region within 270 nucleotides of the start codon of a HSL2 gene (e.g. to a region comprising nucleotides 250-260 of the coding sequence of HSL2, e.g. nucleotides 250-260 of SEQ ID NO: 7 and SEQ ID NO: 10). Thus, the guide RNAs may comprise a first guide RNA comprising a nucleotide sequence of SEQ ID NO: 49 and second guide RNA comprising a nucleotide sequence of SEQ ID NO: 50.

It will be evident that any of the guide RNA sequences specified above may be used alone or in combination with other guide RNA sequences for the same gene, e.g. when more than one guide RNA is used to target a gene, e.g. a HAE gene. Thus, the heritable mutations or modifications may be induced using one guide RNA for a HAE gene and one guide RNA for a HSL2 gene, wherein the guide for the HAE gene may be selected from any one of SEQ ID NOs: 43-46 and the guide for the HSL2 gene may be selected from any one of SEQ ID NOs: 47-50. When the heritable mutations or modifications are induced using more than one guide RNA (e.g. 2 guide RNAs) for one or both genes, they may be independently selected from any of the sequences specified herein, e.g. a first HAE guide RNA comprising SEQ ID NO: 43 and a second HAE guide RNA comprising SEQ ID NO: 46, and a first HSL2 guide RNA comprising SEQ ID NO: 48 and a second HSL2 guide RNA comprising SEQ ID NO: 49. It will be appreciated that any suitable combination of guide RNAs may be used. In some embodiments, it may be advantageous to use all of the exemplified guide RNAs. In some embodiments, the guide RNAs may be at least 98% or 99% identical to the exemplified sequences, e.g. SEQ ID NOs: 43-50.

In a representative embodiment, a mutant or modified Brassica oilseed plant may be produced by introducing a CRISPR-associated nuclease, e.g. Cas9, into a Brassica oilseed plant cell (e.g. a B. napus cell) with a first guide RNA, and optionally a second guide RNA, for each target gene (i.e. HAE and HSL2 gene) as CRISPR-associated nuclease-gRNA complexes (e.g. Cas9-gRNA complexes) that recognize target sequences in the HAE and HSL2 genes and are able to induce DSBs in the genomic sequence. The DSBs may flank a portion of the coding sequences (e.g. a portion of exon 1) in the target genes allowing for the excision of at least a portion of the target genes that disrupts the expression and/or activity of the genes and/or proteins as defined herein.

A CRISPR-associated nuclease, e.g. Cas9, may be introduced into a Brassica oilseed plant cell using any suitable means. As described in the Examples, the CRISPR-associated nuclease may be introduced in the form of a vector encoding the nuclease and optionally also comprising one or more sequences encoding guide RNAs. The CRISPR-associated nuclease and guide RNAs encoded by the vector are expressed in the cell to provide the CRISPR-associated nuclease-gRNA complexes (e.g. Cas9-gRNA complexes).

“CRISPR” (Clustered Regularly Interspaced Short Palindromic Repeats) loci refers to certain genetic loci encoding components of DNA cleavage systems, for example, used by bacterial and archaeal cells to destroy foreign DNA. A CRISPR locus can consist of a CRISPR array, comprising short direct repeats (CRISPR repeats) separated by short variable DNA sequences (called spacers), which can be flanked by diverse Cas (CRISPR-associated) genes.

The terms “Cas protein” refers to a polypeptide encoded by a Cas (CRISPR-associated) gene. A Cas protein includes but is not limited to: a Cas9 protein, a Cpfl (Casl2) protein, a C2cl protein, a C2c2 protein, a C2c3 protein, Cas3, Cas3-HD, Cas5, Cas7, Cas8, CasIO, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. A Cas endonuclease described herein comprises one or more nuclease domains. The endonuclease may have one or more RuvC nuclease domains. A Cas protein may be a functional fragment or functional variant of a native Cas protein, or a protein that shares at least 70%, at least 75%, at least 80%, at least 85%, at least 90%, at least 95%, at least 96%, at least 97%, at least 98%, at least 99%, or 100% sequence identity with a native Cas protein, and retains at least partial activity of the native Cas protein.

A “Cas endonuclease” may comprise domains that enable it to function as a double-strand-break-inducing agent. A “Cas endonuclease” may also comprise one or more modifications or mutations that abolish or reduce its ability to cleave a double-strand polynucleotide (dCas). The Cas endonuclease molecule may retain the ability to nick a single-strand polynucleotide (for example, a D10A mutation in a Cas9 endonuclease molecule) (nCas9).

A “native gene” refers to a gene as found in its natural endogenous location with its own regulatory sequences.

A “mutated gene” or “modified gene” is a gene that has been altered through human intervention. Such a “mutated” or “modified” gene has a sequence that differs from the sequence of the corresponding non-mutated (i.e. native) gene by at least one nucleotide addition, deletion, or substitution as defined elsewhere herein. A mutated gene may comprise an excision or deletion of a sequence of nucleotides within that results from two double strands break which are specifically targeted to a genomic sequence by guide polynucleotide/Cas endonuclease system as disclosed herein. A “mutated” or “modified” plant is a plant comprising a mutated gene, e.g. a gene comprising a deletion.

As used herein, a “targeted mutation” is a mutation in a gene (referred to as the target gene), including a native gene, that was made by altering a target sequence within the target gene using any method known to one skilled in the art, including a method involving a guided CRISPR-associated nuclease (e.g. Cas endonuclease) system as disclosed herein. A recognition site and/or target site can be comprised within an intron, coding sequence (e.g. exon), 5′ UTR, 3′ UTR, and/or regulatory regions (e.g. promoter).

The terms “dropout”, “gene dropout”, “knockout” and “gene knockout” may be used to refer to a DNA sequence of a cell (e.g. a HAE or HSL2 gene) that has been mutated such that it does not produce a functional gene product (e.g. polypeptide). In particular, a dropout or gene dropout refers to a mutation in which part or all of a gene (e.g. a coding sequence or part thereof) is excised from the genome by targeted deletion, e.g. mediated by a CRISPR-associated nuclease.

Thus, the invention may be seen to provide a method comprising introducing a CRISPR-associated nuclease (e.g. Cas9) and one or more guide RNAs into a Brassica oilseed plant cell (e.g. B. napus or B. rapa plant cell) to generate a heritable mutation in a HAE and HSL2 gene, preferably wherein the mutation is a knockout mutation, e.g. frameshift mutation, and subsequently generating a plant from the cell comprising the heritable mutations (i.e. a mutant or modified Brassica oilseed plant as defined herein). The method may further comprise a step of harvesting or obtaining one or more seeds from the plant and optionally selecting seeds comprising the heritable mutations.

As used herein, the term “guide polynucleotide” relates to a polynucleotide sequence that can form a complex with a CRISPR-associated nuclease (e.g. Cas endonuclease) and enables the CRISPR-associated nuclease to recognize, optionally bind to, and optionally cleave a DNA target site. The guide polynucleotide sequence can be an RNA polynucleotide, a DNA polynucleotide, or a combination thereof (an RNA-DNA combination polynucleotide), e.g. a guide RNA. In some embodiments, the guide polynucleotide may be provided as part of a vector that encodes a guide RNA and optionally one or more other components required to achieve a CRISPR-associated nuclease (e.g. Cas9) mediated targeted modification, e.g. the vector may encode a CRISPR-associated nuclease (e.g. Cas9) and one or more additional guide polynucleotides (e.g. guide RNAs).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break in) the DNA target site.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system”, “Polynucleotide-guided endonuclease (PGEN)” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break in) the DNA target site. A guide polynucleotide/Cas endonuclease complex herein can comprise Cas protein(s) and suitable polynucleotide component(s) of any of the known CRISPR systems.

Any suitable methods and compositions for polynucleotide modification with a CRISPR Associated (Cas) endonuclease may be used to generate the mutant or modified plants disclosed herein. Class I Cas endonucleases comprise multi-subunit effector complexes (Types I, III, and IV), while Class 2 systems comprise single protein effectors (Types II, V, and VI). In Class 2 Type II systems, the Cas endonuclease acts in complex with a guide RNA (gRNA) that directs the Cas endonuclease to cleave the DNA target to enable target recognition, binding, and cleavage by the Cas endonuclease. The gRNA comprises a Cas endonuclease recognition (CER) domain that interacts with the Cas endonuclease, and a Variable Targeting (VT) domain that hybridizes to a nucleotide sequence in a target DNA. In some aspects, the gRNA comprises a CRISPR RNA (crRNA) and a trans-activating CRISPR RNA (tracrRNA) to guide the Cas endonuclease to its DNA target. The crRNA comprises a spacer region complementary to one strand of the double stranded DNA target and a region that base pairs with the tracrRNA, forming an RNA duplex. In many systems, the Cas endonuclease-guide polynucleotide complex recognizes a short nucleotide sequence adjacent to the target sequence (protospacer), called a “protospacer adjacent motif” (PAM).

Examples of a Cas endonuclease include but are not limited to Cas9 and Cpfl. Cas9 (formerly referred to as Cas5, Csnl, or Csxl2) is a Class 2 Type II Cas endonuclease. A Cas9-gRNA complex recognizes a 3′ PAM sequence (NGG for the S. pyogenes Cas9) at the target site, permitting the spacer of the guide RNA to invade the double-stranded DNA target, and, if sufficient homology between the spacer and protospacer exists, generate a DSB cleavage. Cas9 endonucleases comprise RuvC and HNH domains that together produce DSBs, and separately can produce single strand breaks. For the S. pyogenes Cas9 endonuclease, the DSB leaves a blunt end. Cpfl is a Class 2 Type V Cas endonuclease, and comprises nuclease RuvC domain but lacks an HNH domain. Cpfl endonucleases create “sticky” overhang ends.

Cas9-gRNA systems may be used to: generate insertions, deletions, substitutions, or modifications of one or more nucleotides at the target site; modify or replace nucleotide sequences of interest (such as a regulatory elements); insert polynucleotides of interest; cause gene dropout or gene knock-out; modify splicing sites and/or introduce alternate splicing sites; modify nucleotide sequences encoding a protein of interest; induce gene silencing by inserting an inverted repeat into a gene of interest or elsewhere in the genome. Genome editing using DSB-inducing agents, such as Cas9-gRNA complexes, has been described and is within the purview of the skilled person.

The disclosed guide polynucleotides can be introduced into a cell with the disclosed CRISPR-Cas endonucleases. Cells include any Brassica oilseed plant cell. In a preferred aspect the cells are B. napus or B. rapa cells.

Standard recombinant DNA and molecular cloning techniques used herein are well known in the art. Vectors and constructs include circular plasmids, and linear polynucleotides, comprising a polynucleotide of interest (e.g. guide polynucleotide) and optionally other components including linker, adapter, regulatory (e.g. promoter, terminator) or analysis (e.g. reporter) sequences.

The constructs disclosed herein may comprise a promoter operably linked to a nucleotide sequence encoding a CRISPR-associated nuclease (e.g. Cas9, e.g. Streptococcus pyrogenes Cas9) gene and a promoter operably linked to a guide polynucleotide (e.g. encoding a guide RNA) of the present disclosure. The promoter is capable of driving expression of an operably linked nucleotide sequence in a prokaryotic or eukaryotic cell/organism. In some aspects, target specific guide RNAs are built as a fusion of CRISPR RNA (crRNA) fused to trans activating CRISPR RNA (tracrRNA), e.g. a Streptococcus pyrogenes tracrRNA.

As noted above, the plant, plant part or seed disclosed herein may be used to breed a plant, grow a crop or produce seed, e.g. further seed, such as for oil extraction. Methods for breeding Brassica oilseed plants, growing crops of such plants and producing or obtaining seed from said plants are well-known in the art and any such methods may be utilised.

The oil derived from Brassica oilseed plants is an important commercial product, e.g. canola oil, along with by-products derived from the extraction and processing procedure. Accordingly, the plants, plant parts and seeds disclosed herein may be used (e.g. processed) to obtain oil, i.e. seed oil (e.g. edible seed oil), and optionally by-products from the oil extraction and/or processing procedure.

Methods of isolating seed oils are well known in the art and any suitable method may be used to obtain oil (e.g. edible oil) from the seeds disclosed herein. Typically, seed oil (e.g. canola oil) is produced using a series of steps involving the extraction and purification of an edible oil product from the oil-bearing seeds. Oils from Brassica oilseed plants (e.g. canola oil from B. napus) and by-products commonly are produced using the generalized steps shown in Table 2.

TABLE 2
Generalized steps for oil and by-product production

| Process | | Impurities Removed and/or |
|---|---|---|
| Step | Process | By-Products Obtained |
| # 1 | Harvesting Brassica | |
| | oilseed plant seed (e.g. | |
| | B. napus seed) | |
| # 2 | oil extraction | meal |
| # 3 | degumming | lecithin |
| # 4 | alkali or physical refining | gums, free fatty acids, pigments |
| # 5 | water washing | soap |
| # 6 | bleaching | color, soap, metal |
| # 7 | (hydrogenation)* | |
| # 8 | (winterization)* | stearin |
| # 9 | Deodorization | free fatty acids, tocopherols, |
| | | sterols, volatiles |
| # 10 | oil products | |

*optional processes

Seeds may be cleaned, tempered, dehulled and/or flaked to increase the efficiency of oil extraction. Oil extraction is usually accomplished by solvent (e.g. hexane) extraction but can also be achieved by a combination of physical pressure and/or solvent extraction. The resulting oil is called crude oil. The crude oil may be degummed by hydrating phospholipids and other polar and neutral lipid complexes that facilitate their separation from the non-hydrating, triglyceride fraction (i.e. the oil fraction, e.g. canola oil). The resulting lecithin gums may be further processed to make commercially important lecithin products which may be used in a variety of food and industrial products as emulsification and release (i.e., anti-sticking) agents. Degummed oil may be further refined for the removal of impurities (primarily free fatty acids, pigments and residual gums). Refining is accomplished by the addition of a caustic agent that reacts with free fatty acid to form soap and hydrates phosphatides and proteins in the crude oil. Water is used to wash out traces of soap formed during refining. The soapstock by-product may be used directly in animal feeds or acidulated to recover the free fatty acids. Colour is removed through adsorption with a bleaching earth that removes most of the chlorophyll and carotenoid compounds. The refined oil can be hydrogenated, thereby resulting in fats with various melting properties and textures. Winterization (fractionation) may be used to remove stearin from the hydrogenated oil through crystallization under carefully controlled cooling conditions. Deodorization (principally via steam distillation under vacuum) is the last step and is designed to remove compounds which impart odour or flavour to the oil. Other valuable by-products such as tocopherols and sterols may be removed during the deodorization process. Deodorized distillate containing these by-products may be sold for production of natural vitamin E and other high-value pharmaceutical products. Refined, bleached, (hydrogenated, fractionated) and deodorized oils and fats may be packaged and sold directly or further processed into more specialized products.

Plant oils containing polyunsaturated fatty acids (PUFAs) that have been refined and/or purified can be hydrogenated to produce fats with various melting properties and textures. Many processed fats (including spreads, confectionary fats, hard butters, margarines, baking shortenings, etc.) require varying degrees of solidity at room temperature and can only be produced through alteration of the source oil's physical properties. This is most commonly achieved through catalytic hydrogenation.

Thus, a method of obtaining the seed disclosed herein is provided, the method comprising:
(a) providing:
(i) a plant or part thereof (e.g. seed pod) as defined herein; or
(ii) a plant produced by the method defined herein; and
(b) harvesting and/or separating seed from the plant or part thereof of (a).

Methods for harvesting seeds from Brassica oilseed plants (e.g. B. napus and B. rapa) and seed pods are well-established in the art and any such method may be used in the method described above, e.g. mechanical harvesting. It will be understood that the harvesting step may not result in an isolated seed product, e.g. unwanted plant parts, such as seed pods or fragments thereof, may be mixed in with the harvested seeds. Thus, the method may involve further steps of separating the seeds from other plant parts. The separation step may be performed contemporaneously with the harvesting step or may be performed as a separate step after the harvesting step. The harvested seed may be further processed to produce oil and/or associated products, e.g. by-products of the oil extraction process.

Thus, a method of obtaining oil is provided comprising:
(a) providing seed as defined herein, e.g. seed obtained from the method of obtaining seed defined herein; and
(b) extracting oil from the seed of (a).

It will be understood that the method of obtaining oil may include the method of obtaining the seed disclosed herein as a step in the oil obtaining method.

As discussed above, the method of obtaining the oil may comprise further processing steps, such as processing the oil extracted from the seed to produce a processed oil or oil product (e.g. an oil fraction containing one or more fatty acids, e.g. one or more polyunsaturated fatty acids, an edible food product etc.) and/or one or more by-products (e.g. meal, lecithin, free fatty acids, pigments, soap, stearin, tocopherols and/or sterols). Thus, the further processing steps may include one or more of degumming (e.g. degumming crude oil), refining (e.g. alkali and/or physical refining), washing, bleaching, hydrogenation, winterization and/or deodorization.

The present invention may be more fully understood from the non-limiting Examples below with reference to the following drawings.

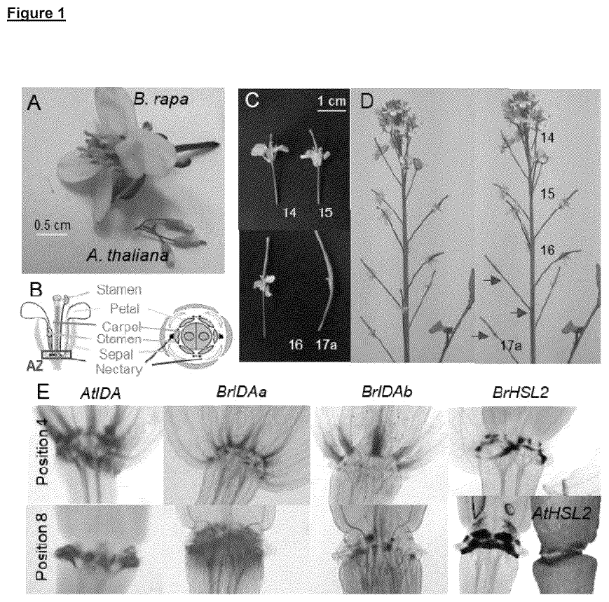

FIG. 1: Floral organ abscission proceeds similarly in B. rapa and A. thaliana. (A) B. rapa flowers are substantially larger than A. thaliana flowers. (B) Abscission zones (AZ) are situated at the base of the floral organs which are organized in whorls organs, here depicted with longitudinal section and cross section of A. thaliana flower. (C) B. rapa R-o-18 flowers with developing fruits at floral stages 14 to 17a. At stage 16 the fruit with the developing seeds has doubled in length, organs start to crumble, and detach with the lightest touch. At stage 17a all organs have abscised. (D) Stage 16 corresponds to flower positions 16-18, when counting from the top of the inflorescence. Here the organs detach by the lightest touch (arrows, compare left and right image). (E) β-glucuronidase (GUS) expression driven by the promoters of the indicated B. rapa and A. thaliana genes in the AZ region of A. thaliana position 4 and 8, corresponding to stages 14 and 17.

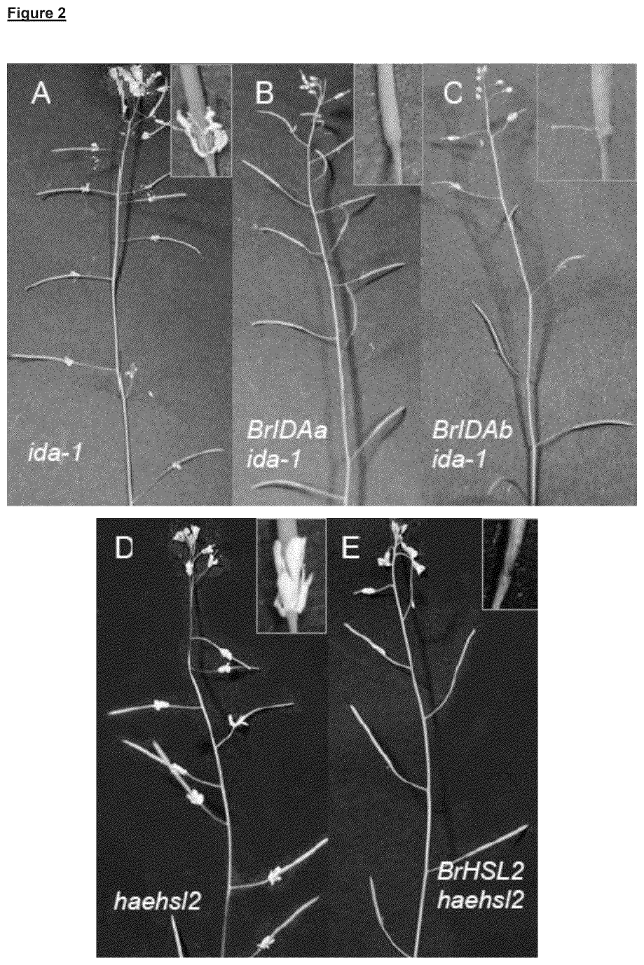

FIG. 2: B. rapa orthologues rescue the abscission deficiency of A. thaliana ida and haehsl2 mutants. (A) and (D) The ida-1 mutant and the haehsl2 double mutant retain their floral organs indefinitely. (B, C and E) Introduction of B. rapa IDA and HSL2 genes (including their promoters) in the respective mutants as indicated, restores the abscission phenotype. Note occasional stamens remaining attached in ida-1 plants transformed with the BrIDAb construct.

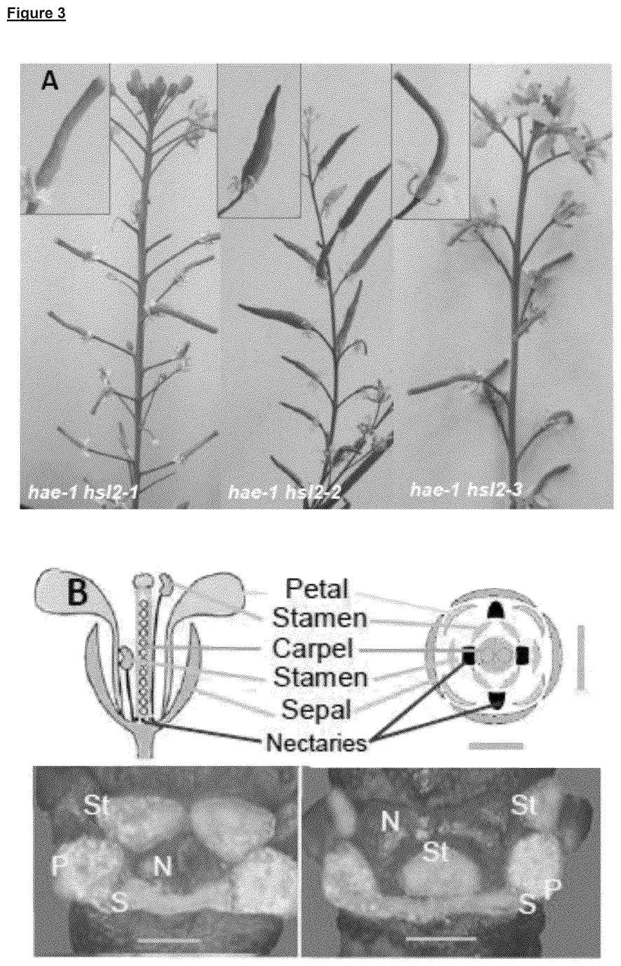

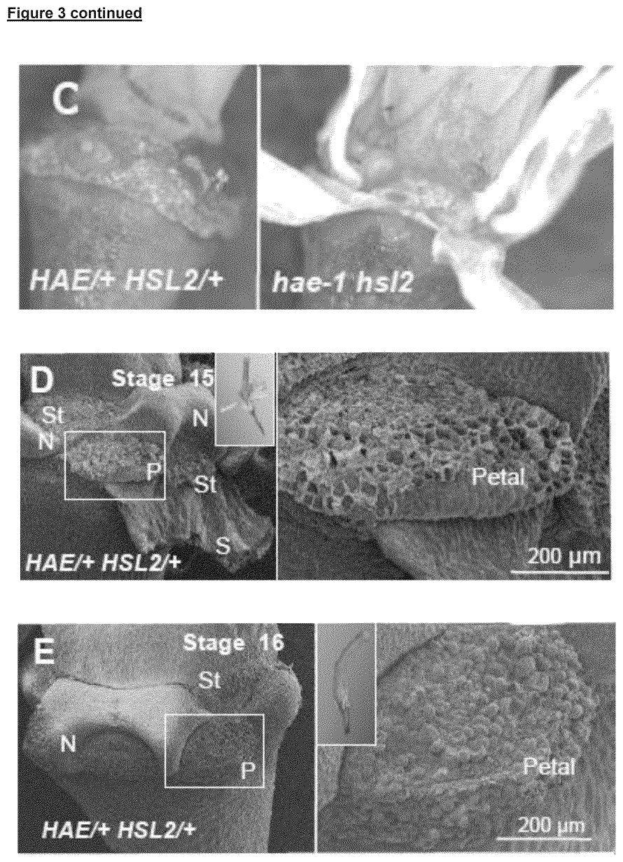

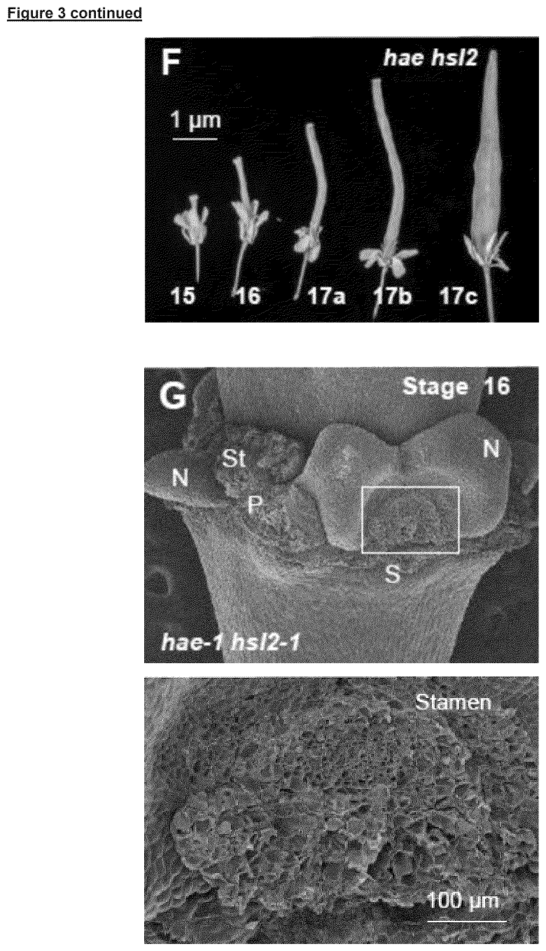

FIG. 3: The brhaehsl2 mutants are deficient in floral organ abscission. (A) Progeny of three independent brhaehsl2 double mutants showing deficiency in floral organ abscission. (B) Longitudinal section and cross section of B. rapa flower, and images of the abscission zone (AZ) region seen from two different angles, cf. green and orange lines in cross section. (C) The floral organ and AZ architecture in brhaehsl2 mutant equal that of B. rapa R-o-18 HAE/+ HSL2/+ plants. (D) and (E) Scanning Electron Micrograph (SEM) of AZs of HAE/+ HSL2/+ plants at stage 14 and stage 16, respectively, cf. miniature images of staged flowers. (F) brhaehsl2 fruits retains floral organs indefinitely, long past floral stage 16 when wild type plants shed these organs. (G) SEM of AZs of brhaehsl2 plants at stage 16. S—sepal; P—petal; St—stamen; N—nectaries.

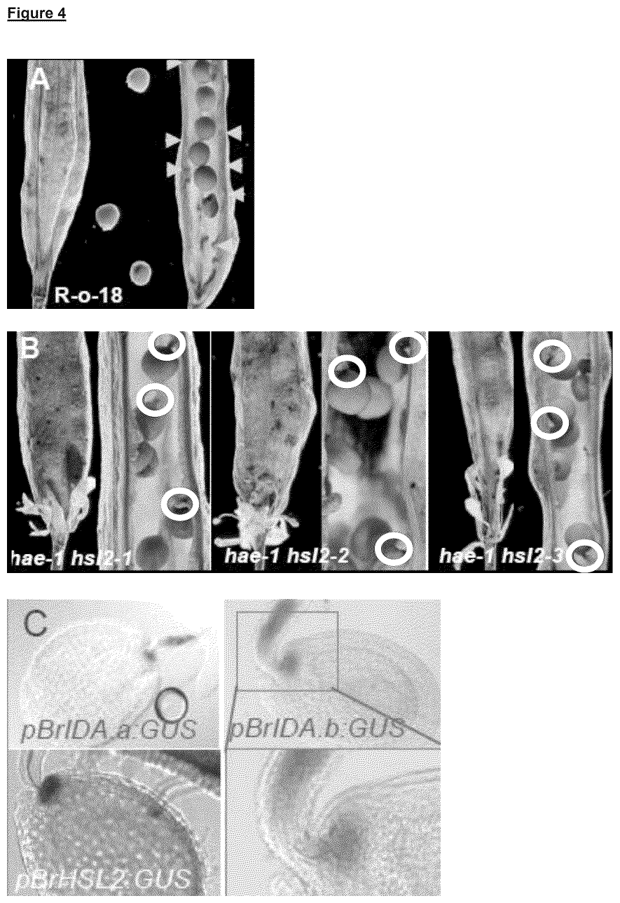

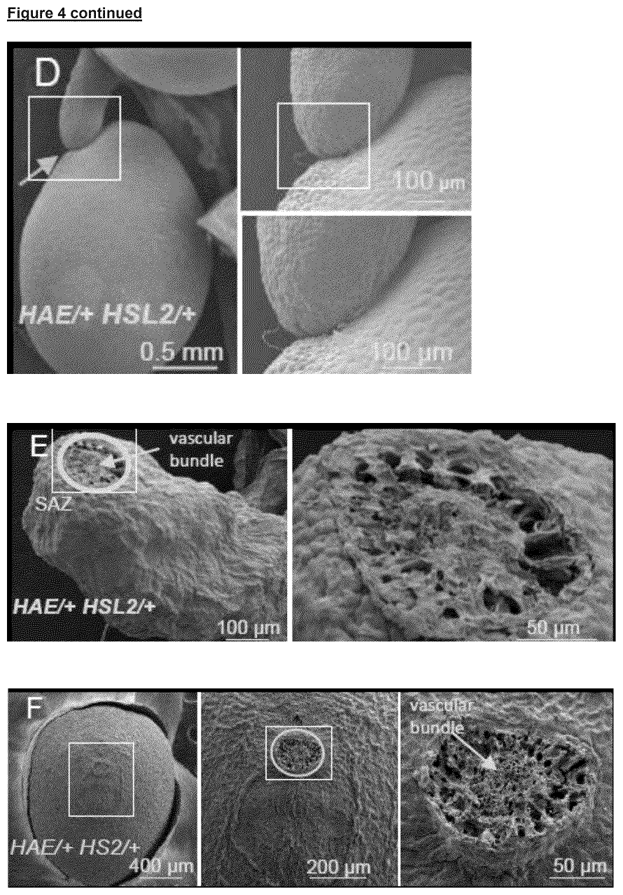

FIG. 4: BrHAE/BrHSL2 control seed detachment in B. rapa. (A) B. rapa R-o-18 closed fruit, and opened fruit with detached seeds. Yellow arrowheads point to empty seed sticks. (B) Seeds of the three independent brhaehsl2 mutants still attached to the seed stick (circles). Also note attached floral organs. (C) pBrIDAa:GUS, pBrIDAb:GUS, and pBrHSL2:GUS expression in A. thaliana seed/seed stick attachment region. (D) SEM of B. rapa R-o-18 HAE/+ HSL2/+ seed stick with attached seed prior to abscission. Orange arrow points to the constricted seed abscission zone (SAZ) where separation takes place. (E) SEM of a mature B. rapa R-o-18 HAE/+ HSL2/+ seed stick after natural seed detachment, with a close-up of the SAZ. (F) SEM of mature B. rapa R-o-18 HAE/+ HSL2/+ seed after natural detachment from the seed stick, with close-ups of the SAZ. (G) SEM of brhaehsl2 mature seed still attached to the seed stick and with intact constricted SAZ (orange arrow), however with a broken seed coat (white arrow). (H) SEM of brhaehsl2 seed stick from mature fruit with a fragment of seed coat (white arrow) still attached via intact constricted SAZ (orange arrow). (I) and (J) SEM of aberrant detachment sites in the seed coat of mature brhaehsl2 seeds that had been forcibly removed from the seed stick.

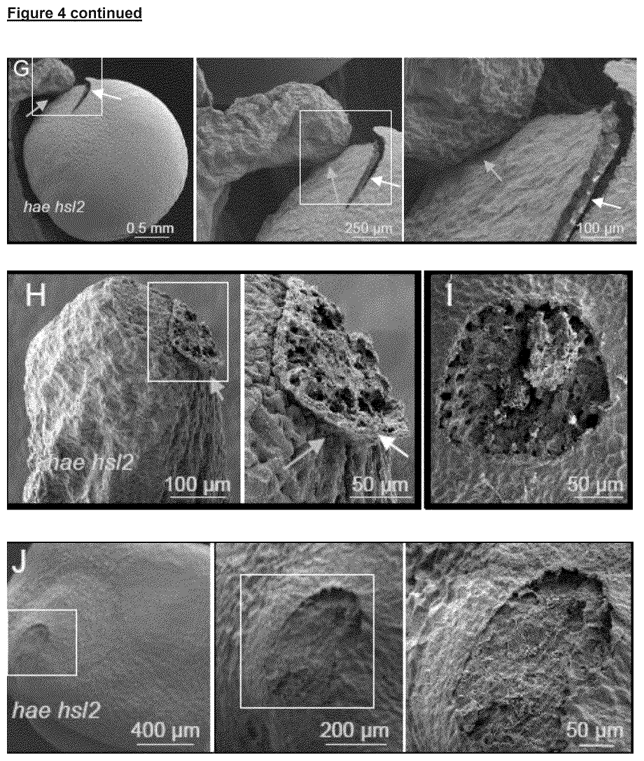

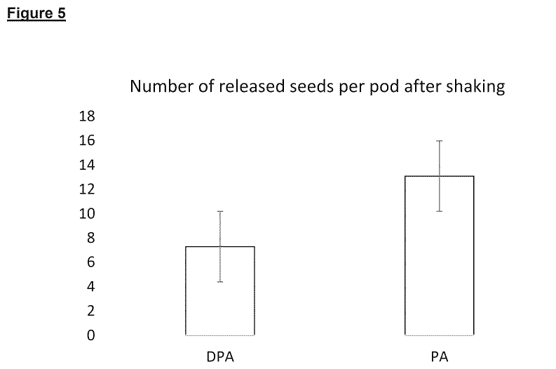

FIG. 5: Bar chart showing the number of seeds released per pod from seed pods harvested from DPA and PA plants following 15 seconds of shaking.

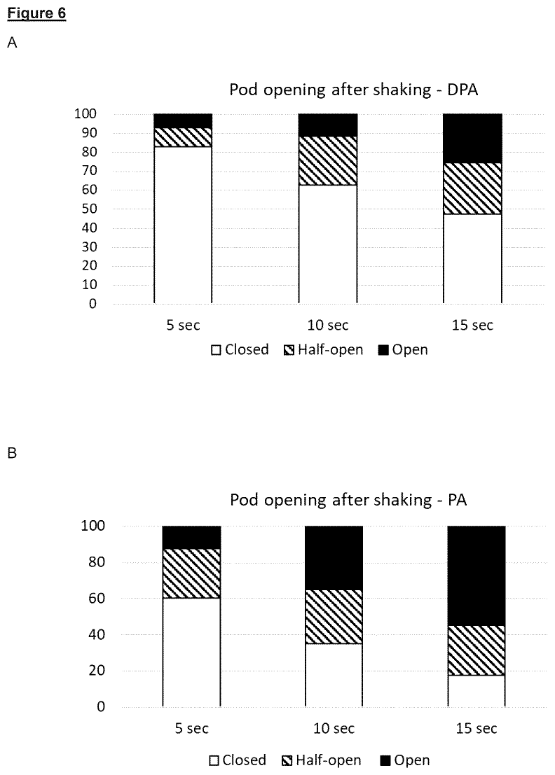

FIG. 6: Bar charts showing the percentage of open, half-open and closed seed pods from (A) DPA and (B) PA plants following shaking for 5, 10 or 15 seconds.

FIG. 7: Diagrams illustrating: (A) the domains of the receptor proteins encoded by the HSL2 and HAE genes; (B) the location of the sites in the encoded proteins targeted by the guide-RNAs exemplified in Example 5 and Table 5; and (C) the location of in-frame deletions in the HSL2-C and HSL2-A proteins observed in the 126 and 206 T1 lines. The scale bar on the right side of each diagram represents 100 amino acids.

### EXAMPLES

### Example 1—B. rapa Orthologues Rescue Abscission Deficient A. thaliana Mutants

IDA belongs to a small gene family of closely related IDA-LIKE (IDL) genes encoding prepropeptides with the typical structure of post-translationally modified plant signalling peptides, namely an N-terminal secretion signal directing the propeptide to the endoplasmic reticulum, where the so-called variable region is removed to release the C-terminally located, bioactive peptide into the apoplastic space. The IDA/IDL peptide family, with members identified in all orders of angiosperms, is defined by the highly conserved 12 amino acid long PIP motif. Brassicale orthologues of each of the A. thaliana IDA/IDL family members represent a monophyletic clade (Table 3). B. rapa and A. thaliana are so closely related that the variable regions also share striking sequence similarity. Thus, two orthologues of AtlDA were easily identified; BraA.IDA.a encoding a PIP motif identical to Arabidopsis IDA, and BraA.IDA.b (hereafter BrIDA.a and BrIDA.b) with two amino acid substitutions (position 1 and 9).

Phylogenetic investigations have previously identified orthologues of HSL2 and the closely related HSL1 in both monocots and dicots, while HAE split off from HSL1 during the evolution of eudicots. BLASTp searches with the A. thaliana receptor proteins as queries combined with phylogenetic analyses revealed that there are only single orthologues for HAE and HSL2, but three HSL1 genes (Table 1), exemplifying the extensive genome reshuffling that can occur after genome duplications. The genes encoding the BraA.HAE.a (A01p009890) and BraA.HSL2.a (A09p010050) orthologues, (hereafter BrHAE and BrHSL2) respectively, share more than 80% identity with their Arabidopsis counterparts including amino acids involved in ligand and co-receptor binding.

TABLE 3
IDA/IDL, HAE and HSL2 orthologues in Brassica rapa

| | | | | CDS position in R-o-18 | | |
|---|---|---|---|---|---|---|
| | | | | chromosome v2.4 | | |
| A. thaliana gene | B. rapa functional gene name | | | start pos | end pos | strand |
| IDA (AT1G68765) | BraA.IDA.a | A07p042260 | LF | 22 920 481 | 22 920 710 | − |
| | BraA.IDA.b | A02p022480 | MF1 | 10 702 235 | 10 702 456 | − |
| IDL1 (AT3G25655) | BraA.IDL1.a | A06p051280 | LF | 26 977 661 | 26 977 933 | + |
| | BraA.IDL1.b | A02p048750 | MF1 | 30 299 093 | 30 299 365 | − |
| IDL2 (AT5G64667) | BraA.IDL2.a | A06p038720 | LF | 20 827 553 | 20 827 570 | + |
| | BraA.IDL2.b | A09p009620 | MF2 | 4 948 598 | 4 951 317 | + |
| IDL3 (AT5G09805) | BraA.IDL3.a | A10p034640 | LF | 20 100 857 | 20 101 172 | − |
| | BraA.IDL3.b | A03p004370 | MF1 | 1 845 523 | 1 845 837 | + |
| | BraA.IDL3.c | A02p003640 | MF2 | 1 663 463 | 1 663 690 | − |
| IDL4 (AT3G18715) | BraA.IDL4.a | A05p040440 | LF | 24 731 582 | 24 731 863 | − |
| | BraA.IDL4.b | A01p047480 | MF1 | 26 775 208 | 26 775 495 | − |
| IDL5 (AT1G76952) | BraA.IDL5.a | A07p048590 | LF | 26 353 783 | 26 354 103 | − |
| | BraA.IDL5.b | A07p034770 | MF2 | 18 859 704 | 18 860 012 | + |
| HAE (AT4G28490) | BraA.HAE.a | A01p009890 | LF | 4 797 771 | 4 800 370 | − |
| HSL2 (AT5G65710) | BraA.HSL2.a | A09p010050 | MF2 | 5 149 620 | 5 152 684 | − |

B. rapa R-o-18 plants and flowers are much larger than A. thaliana plants (FIG. 1A), but these two crucifer species have similar organ architecture in the whorls around the carpel, with four sepals, four petals, two short and four long stamen with nectaries in between (FIG. 1B). In these two species the floral organs abscise from the mother plant at about the same developmental stage, namely stage 16, when the fruit with the developing seeds has elongated well beyond the long stamen, and the petals have started to crumble (FIG. 1C). At stage 17a in B. rapa all organs have been lost. Counting from the top of an A. thaliana inflorescence in full bloom, this stage is reached at position 6-8 depending on the ecotype. In B. rapa, this corresponds to position 16-18, where the floral organs detach by the lightest touch (FIG. 1D). Thus, it can be seen that B. rapa R-o-18 displays similar floral organ abscission to A. thaliana.

The high similarity between A. thaliana and B. rapa floral organ abscission, and high amino acid identity of B. rapa receptors and peptide ligands with their Arabidopsis counterparts, raised the question of whether the Brassica promoters would work in Arabidopsis. To address this question, promoter: β-glucuronidase (GUS) constructs for BrIDAa, BrIDAb and BrHSL2 were generated and introduced into Arabidopsis ecotype Col-0 using Agrobacterium tumefaciens.

In A. thaliana, IDA and its receptors are expressed in the AZs at the base of the floral organs to be shed. The B. rapa GUS plants displayed spatial and developmental patterns of expression in the floral organs highly similar to the respective Arabidopsis promoter:GUS constructs, with appearance from position 4, increased expression level until position 8, when the organs have been shed, and thereafter gradual disappearance (FIG. 1E). Thus, the Brassica promoters are recognized and transcribed as if they were part of the transcriptome of the Arabidopsis AZ regions.

### Example 2—B. rapa Orthologues Rescue the Abscission Deficient ida and haehsl2 Mutants

To provide a sensitive and comprehensive test of the functionality of the Brassica genes, the two B. rapa IDA genes and the BrHSL2 gene, including their promoters, were introduced in the A. thaliana ida-1 mutant and haehsl2 double mutant, respectively (FIG. 2). BrIDAa and BrHSL2 fully restored the wild type phenotype with abscising sepals, petals and stamens (FIGS. 2B and 2E), while in lines transformed with BrIDAb some stamens remained attached (FIG. 2C). Whilst not wishing to be bound by theory, it is thought that this may indicate that the substitution of the positively charged lysine (Lys, K) in position 9 of the peptide with an uncharged Asparagine (Asn, N) to some extent reduces the bonding ability of the peptide to a receptor or co-receptor, and/or that the processing to release a mature peptide from the propeptide is affected by the change of the position 1 proline (Pro, P) to a serine (Ser, S).

### Example 3—Floral Organ Abscission in B. rapa is Controlled by HAE and HSL2

To directly address whether IDA-HAE/HSL2 signalling controls cell separation events in B. rapa, three independent haehsl2 double mutants were generated from a B. rapa tilling collection and studied. In the B. rapa tilling collection (made in the R-o-18 line) at the John Innes Centre, Norwick, UK, three lines heterozygous for alleles with premature stop codons in the BrHSL2 coding region and one with a stop codon in the BrHAE gene were identified (Table 4). The three BrHSL2 lines were all crossed with the BrHAE line, and F2 progeny plants were screened for a floral abscission-deficient phenotype. Such plants were identified in progeny from all three crosses, and genotyping confirmed that they, as expected, were brhaehsl2 double mutants. Plants without abscission deficiency were all genotyped to be wild type or heterozygous for one or two of the mutant receptor genes. The progeny of the double mutants consistently showed the same abscission deficient phenotype (FIG. 3A). Thus, as in Arabidopsis, the two receptors are redundant in function with respect to floral organ abscission.

The brhaehsl2 mutants showed the same organ organization in the AZ region as B. rapa R-o-18, however, without loss of floral organs (FIG. 3B-C). In fertilized R-o-18 flowers, stage 15, force needs to be applied to remove sepals, petals and stamen, and this results in tom and disrupted cells in the abscission zone (FIG. 3D). This is in sharp contrast to stage 16, when the floral cell separation takes place. As in Arabidopsis, scanning electron microscopy (SEM) documented that rounded, enlarged cells develop at the wild type surface (FIG. 3E).

Both in Arabidopsis and B. rapa the haehsl2 mutant organs remain attached throughout the development of the fruits (FIG. 3F), and at any time point removal of the organs will display broken cells (FIG. 3G). This indicates that pectineus middle lamellae degradation allowing wild type organs to detach without cellular damage is impaired in the mutant. Thus, blocking of IDA-HAE/HSL2 signalling results in highly similar floral mutant phenotypes in Arabidopsis and B. rapa.

TABLE 4
Premature stop codon-mutations in HAE and HSL2 in B. rapa tilling lines

| HSL2 - A09p010050 | | | | HAE - A01p009890 | |
|---|---|---|---|---|---|
| Line | JI30115-A | JI30834-B | JI30925-B | Line | JI30272-B |
| Chromosome | A09 | A09 | A09 | Chromosome | A01 |
| Position | 3 791 884 | 3 792 614 | 3 792 894 | Position | 4 333 978 |
| WT base | G | G | G | WT base | G |
| MT base | A | A | T | MT base | A |
| Type | het | het | het | Type | het |
| WT coverage | 20 | 13 | 17 | WT coverage | 20 |
| MT coverage | 16 | 18 | 15 | MT coverage | 14 |
| Effect | stop | stop | stop | Effect | stop gained |
| | gained | gained | gained | | |
| Codon | tgG/tgA | tGg/tAg | Gag/Tag | Codon | Caa/Taa |
| Amino acid | Trp428/* | Trp645/* | Glu772/* | Amino acid | Gln651/* |
| Nucleotide | 1284 G > A | 1934 G > A | 2314 G > T) | Nucleotide | 1951 C > T |

### Example 4—Seed Abscission in B. rapa is Controlled by HAE and HSL2

In B. rapa line R-o-18 seeds detach between stage 17b and 17c, which is prior to pod opening, a process called dehiscence (FIG. 4A). At the margins of the valves there are dehiscence zones (DZs) with a lignified layer and a separation layer. The latter is weakened by cell wall remodelling enzymes, like polygalacturonases (PGs), reminiscent of abscission. When the pod dries, tension builds up, eventually leading to splitting at the separation layer. However, when harvesting the three B. rapa haehsl2 lines, it was discovered that the seeds remained attached even when the pods were completely dry (FIG. 4B). Inspection of the Arabidopsis lines transgenic for the BrIDAa, BrIDAb and BrHSL2 promoter:GUS constructs indicated that these Brassica genes normally are expressed at the tip of the seed stick (funiculus) and at the attachment site of the seed (FIG. 4C).

Using SEM, it was found that the separation of the B. rapa seed from the seed stick takes place in a layer which is constricted relative to the rest of the seed stick and the seed (orange arrow, FIG. 4D). The seed stick diameter reduces strongly (about 50-60%) in the very last μm before attaching to the seed. In R-o-18 natural detachment of the seed revealed a circular scar with a bundle of vasculature at the centre surrounded by 1-2 circles of cells, both on the plant side and the seed side (FIG. 4E-F), as expected from splitting the cells in the constricted separation layer. In analogy to dehiscence, it is hypothesized that the cell wall in the separation layer has been weakened by cell wall loosening enzymes, and that the layer therefore will burst when the seed stick and the seed dries out. In contrast to floral organ abscission with rounded cells and generation of a layer that protects the plant against intruders, such protection is not needed when the seeds are ripe.

In the brhaehsl2 mutants, the constriction was also present (arrow, FIG. 4G-H), however, after removal of the seed a variety of breaks were observed in the seed coat and uneven surfaces were found both on the seed stick and the seed side (white arrows, FIG. 4G-J). Such phenotypes are to be expected upon removal of seeds if the walls of the separation layer cells fail to undergo cell wall weakening. Thus, it is concluded that HAE and HSL2 control not only floral organ abscission but also seed detachment in Brassica oilseed plants, such as B. rapa.

### Example 5—Generation of B. napus Haehsl2 Mutants

B. napus contains two orthologues (i.e. copies) of the HAE and HSL2 genes, terms A and C respectively, which are derived from B. rapa and B. oleracea. It is hypothesised that disrupting only the B. rapa copies (A copies) of the HAE and HSL2 gene may be sufficient to confer the seed retention phenotype observed in the B. rapa haehsl2 mutant. However, as the A and C copies of the target genes HSL and HAE2 share regions of sequence similarity, it was determined that these regions could be targeted for mutation simultaneously (i.e. CRISPR-associated nuclease mediated targeting) to generate plants with mutations in all four genes. Subsequently, plants containing different combinations of mutations, e.g. in both A genes, in both C genes, in both A and C genes, could be isolated and characterised to determine their level of seed retention relative to unmodified plants.

The genomic DNA sequences of the A and C copies of the target genes HAE and HSL2 were aligned using the software Geneious Prime. Homologous regions of each gene were selected, and guide polynucleotides chosen that would target both A and C gene copies. Four guide polynucleotides were designed for each gene (Table 5). The selected guide polynucleotides were ordered as forward and complementary oligonucleotides with 5′ overhangs to enable cloning. Using standard Golden Gate MoClo assembly the guide polynucleotides were cloned between the U6-26 promoter and the guide scaffold for Cas9 in the Level 1 acceptor plasmids pICSLIP3-AtU626, pICSLIP4-AtU626, pICSLIP5-AtU626, and pICSLIP6-AtU626 in positions 3, 4, 5 and 6 respectively. These level 1 plasmids were sequenced, before proceeding to the Level 2 assembly. Level 2 assembly was performed using the Level 2 acceptor pGoldenGreenGate-M (pGGG-M) binary vector. The level 1 plasmids pICSL11137L1P1-2×35s::NptII::NosTerm (selectable marker), pICSL11129-AtUbi10P::SheenCas9::E9Term (Cas9) and the guide cassettes were assembled into pGGG-M along with end linker pICSL41822. Two level 2 vectors were produced each containing 4 guides, 2 guides targeting the HSL2 gene copies and 2 guides targeting the HAE gene copies. The resulting plasmids were deemed pGGG-Oslo 1 containing guides HAEg1, HSLg2, HAEg3, and HSLg4, and pGGG-Oslo 2 containing guides HSLg1, HAEg2, HSLg3 and HAEg4. The plasmids were sequenced to ensure their authenticity before transferring to Agrobacterium for transformation of B. napus cells.

TABLE 5
gDNAs used to produce haehsl2 mutants in B. napus

| | | | SEQ |
|---|---|---|---|
| | | | ID |
| Gene | gRNAs in Construct | gRNAs' Sequence 5′-3′ | NO: |
| HAE | gRNA1_pGGG-Oslo 1 | 5′ AAGAGAGGCATAGGAGGAGGAGG 3′ | 43 |
| | gRNA2_pGGG-Oslo 1 | 5′ AGCCCAGTTTGGCTTGCCGGAGG 3′ | 44 |
| | gRNA3_pGGG-Oslo 2 | 5′ GGACCAGGAAGATAGTGACTGGG 3 | 45 |
| | gRNA4_pGGG-Oslo 2 | 5′ GTCACTCCGTGCCACTGGCGCGG 3′ | 46 |
| HSL2 | gRNA1_pGGG-Oslo 2 | 5′ GGGTCATAACCGGAGATAACCGG 3′ | 47 |
| | gRNA2_pGGG-Oslo 2 | 5′ GTGATTCCAGTCCAGTTACAGGG 3′ | 48 |
| | gRNA3_pGGG-Oslo 1 | 5′ GCAATAGATCTCTCCGACTACGG 3′ | 49 |
| | gRNA4_pGGG-Oslo 1 | 5′ TCTCCGGTGGGTTCCCGTACGGG 3′ | 50 |

The hypervirulent Agrobacterium tumefaciens strain AGL1 was used in all plant transformation experiments. Vectors were electroporated into Agrobacterium AGL1 competent cells and were co-electroporated with the helper plasmid pSoup. Single colonies of Agrobacterium AGL1, which contain the desired vector, were inoculated into 10 mL of LB liquid medium containing appropriate antibiotics and incubated at 28° C., shaken at 200 rpm for ~65 h. Equal quantities of 30% sterile glycerol and the Agrobacterium culture were mixed by inverting and aliquots of 400 μL in 0.5 mL Eppendorf tubes were made. The aliquots of standard inoculums were frozen at −80° C. and stored until required.

B. napus hypocotyl transformation using the method disclosed in Dai et al., 2020 (Mol. Breeding, 40:96, incorporated herein by reference) was performed using the Agrobacterium inoculums described above. Mutant B. napus plants comprising a mutation in at least one HAE or HSL2 gene were generated and characterised.

### Example 6—Characterisation of B. napus Haehsl2 Mutants

The T0 generation of B. napus plants generated as described in Example 5 were initially screened using a “rattling” test to identity plants that may contain mutations in one or more of the HAE and HSL2 genes.

The rattling test is based on the typical developmental process of B. napus seed pods. During development in wild-type plants, green pods and seeds change colour to yellow and black, respectively. At some point during this process, the mature seeds are released from the seed stick and a rattling sound can be heard when shaking the pods near the ear. Thus, a weak rattling phenotype is indicative that the seeds have not been released from the seed stick and the plants may have a deficiency/delay in seed abscission.

Forty T0 plants displayed weak rattling and were screened for mutations in the target regions of the target genes. Notably, thirteen of the transformed TO plants with a weak rattling phenotype were deficient in petal abscission (DPA), i.e. the plants did not shed petals, which is consistent with the hypothesis that the same receptors are involved in different cell-separation processes.

### Genotyping of Plants

Genotyping, using DNA isolated from the 3rd true leaf of the plants, indicated that the DPA T0 plants contained mutations in three or four of the target genes in the guide target regions. As expected, mutations in T0 plants in the guide target regions were also present in the progeny, however, numerous additional mutations, e.g. deletions stretching over two or three target sequences, short deletions (2-9 bp) and point mutations, were detected in subsequent genotyping of different leaves of the same plants and the T1 progeny. These results indicate that the CRISPR-construct used the generate the mutant plants was very active and generated mutations both later during T0 plant growth and in the next generation.

Seeds of the T0 plants were transferred to the Department of Biosciences, University of Oslo, and six T0 plants (251a, 118, 126, 20, 34, 206) were selected to generate T1 plants for further characterization. The T0 plants were selected based on phenotypic and genotypic characteristics (deficiency in petal abscission, nptII copy number, number of mutations in target genes). Twenty-four T1 progeny plants germinated from seeds of the selected T0 plants were genotyped for mutations in the target genes. Table 6 below summarizes the mutations identified in the target genes of the selected T1 plants. As all 6 lines (251a, 118, 126, 20, 34, 206) displayed the DPA phenotype, they are referred to herein as “DPA plants”.

TABLE 6
Mutations identified in the target genes of selected T1 plants

| Line | Genes and types of mutations identified | | | | Location and expected |
|---|---|---|---|---|---|
| (phenotype) | HSL2-A | HSL2-C | HAE-A | HAE-C | effect of mutations |
| 118 (DPA) | Large del, | 8 bp del | Numerous | Numerous | Mutations in regions targeted |
| | 174 bp; | | indels and | indels and | by guide RNA; all four proteins |
| | point muts | | muts | muts | expected to be dysfunctional. |
| 20 | Large del, | 1 bp ins | Dels and | Numerous | Mutations in regions targeted |
| (DPA) | 86 bp; | and muts | muts | indels and | by guide RNA; all four proteins |
| | and indels | | | muts | expected to be dysfunctional. |
| 34 | 2 bp del, | 2 bp del, | 5 bp del, | 2 bp del | Mutations in regions targeted |
| (DPA) | several | dels and | indels and | | by guide RNA; all four proteins |
| | indels and | muts | muts | | expected to be dysfunctional. |
| | muts | | | | |
| 251a | 7 bp del | 5 bp del, | 26 bp del, | Several large | Mutations in regions targeted |
| (DPA) | | dels and | and | dels and muts | by guide RNA; all four proteins |
| | | muts | numerous | | expected to be dysfunctional. |
| | | | indels and | | |
| | | | muts | | |
| 206 PA | Numerous | 3 bp in- | 2 times 1 bp | Large del, 77 | A 3 bp in-frame mutation in |
| (PA) | indels and | frame del | del | bp | HSL2-C is in a region between |
| | muts | | | | the encoded N-terminal |
| | | | | | secretion signal and the start |
| | | | | | of leucine-rich repeats. The |
| | | | | | HSL2-C gene is expected to |
| | | | | | encode a functional protein |
| | | | | | and results in the Petal |
| | | | | | Abscission (PA) phenotype. |
| | | | | | The HSL2-A, HAE-A and |
| | | | | | HAE-C genes contain |
| | | | | | mutations in regions targeted |
| | | | | | by guide RNA and the proteins |
| | | | | | encoded by these are |
| | | | | | expected to be dysfunctional. |
| 206 | Numerous | 3 bp in- | 2 times 1 bp | Large del, 77 | The HSL2-A, HAE-A and |
| (DPA) | indels and | frame del, | del | bp | HAE-C genes contain the |
| | muts | muts and | | | same mutations as line 206 |
| | | dels | | | PA, i.e. mutations in regions |
| | | | | | targeted by guide RNA and |
| | | | | | the proteins encoded by these |
| | | | | | genes are expected to be |
| | | | | | dysfunctional. |
| | | | | | The HSL2-C gene contains |
| | | | | | additional mutations relative to |
| | | | | | the gene in line 206 PA. The |
| | | | | | additional mutations are in |
| | | | | | regions targeted by guide |
| | | | | | RNA and are expected to |
| | | | | | result in a dysfunctional |
| | | | | | protein. Thus, all four proteins |
| | | | | | expected to be dysfunctional |
| | | | | | resulting in plants Deficient in |
| | | | | | Petal Abscission (DPA). |
| 126 PA | 9 bp in- | Several | >20 bp del | Several | A 9 bp in-frame mutation in |
| (PA) | frame | dels and | | indels | HSL2-A is in a region between |
| | deletion | muts | | | the encoded N-terminal |
| | | | | | secretion signal and the start |
| | | | | | of leucine-rich repeats. The |
| | | | | | HSL2-A gene is expected to |
| | | | | | encode a functional protein |
| | | | | | and results in the Petal |
| | | | | | Abscission (PA) phenotype. |
| | | | | | The HSL2-C, HAE-A and |
| | | | | | HAE-C genes contain |
| | | | | | mutations in regions targeted |
| | | | | | by guide RNA and the proteins |
| | | | | | encoded by these genes are |
| | | | | | expected to be dysfunctional. |
| 126 | 9 bp in- | Several | >20 bp del | Several | The HSL2-C, HAE-A and |
| (DPA) | frame | dels and | | indels | HAE-C genes contain the |
| | deletion, | muts | | | same mutations as line 126 |
| | muts | | | | PA, i.e. mutations in regions |
| | | | | | targeted by guide RNA and |
| | | | | | the proteins encoded by these |
| | | | | | genes are expected to be |
| | | | | | dysfunctional. |
| | | | | | The HSL2-A gene contains |
| | | | | | additional mutations relative to |
| | | | | | the gene in line 126 PA. The |
| | | | | | additional mutations are in |
| | | | | | regions targeted by guide |
| | | | | | RNA and are expected to |
| | | | | | result in a dysfunctional |
| | | | | | protein. Thus, all four proteins |
| | | | | | expected to be dysfunctional, |
| | | | | | resulting in plants Deficient in |
| | | | | | Petal Abscission (DPA). |

Key: Del—deletion; Ins—Insertion; indels—insertions and deletions; muts—point mutations; DPA—plants are deficient in petal abscission; PA—plants display petal abscission.

The results from the genotyping analysis demonstrate that each of the genes in lines 251a, 118, 126, 20, 34 and 206 contained mutations affecting the N-terminal portion of the encoded receptor proteins thus making it highly likely that the disruption leads to a non-functional protein (see FIG. 7B, which shows the regions of the proteins that were targeted in the encoding genes by the guide RNAs). While the initial genotypic analysis did not confirm that the plants are homozygous for all of the identified mutations, in view of the persistent activity of the CRISPR-construct used to generate the plants, it is expected that each allele of each gene contains at least one mutation that will result in a decrease in protein function/activity. Thus, the selected T1 plants were all quadruple mutants and may be considered to be equivalent or comparable to homozygous quadruple mutants.

Notably, putative CRISPR-free plants were identified by PCR in four of the six T1 progeny (progeny of 251a, 20, 34 and 206), using primers for the nptIII marker gene of the CRISPR T-DNA construct. These plants may be used to generate subsequent generations for further characterization of the mutations, including segregating populations which will facilitate the assessment of each of the mutations alone and in combination with each other, e.g. to assess the effects of single, double, triple and quadruple mutants, including the quantitative effects of each mutation.

As outlined in Table 6, lines 126 and 206 contained in-frame deletions in HSL2-C or HSL2-A, respectively. T1 plants that contain only these in-frame mutations in HSL2-C or HSL2-A were identified and it was found that these plants displayed petal abscission (PA) and seed abscission characteristics akin to wild-type plants. These lines were termed 126PA and 206PA and are referred to herein as “PA plants”. As the mutations result in the deletion of amino acids in regions of the proteins that are not considered to be key functional domains (see FIG. 7C), it is hypothesised that these in-frame deletions do not disrupt protein function. In this respect, the genes encode proteins that have four important regions: 1) the N-terminal secretion signal needed to position the receptor in the cell membrane; 2) the leucine-rich repeat extracellular region which interacts with signaling peptides; 3) the transmembrane region; and 4) the intracellular kinase domain. The in-frame deletions in lines 206 and 126 are found in the regions between 1) and 2). The conclusion that the in-frame deletions do not disrupt protein function is consistent with the observed petal abscission and seed abscission phenotype and the observation that plants with the same deletion but with additional point mutations expected to disrupt protein function were deficient in petal abscission (DPA).

As the PA plants displayed phenotypes akin to wild-type plants they were used as internal controls in investigations related to seed retention (see below). Moreover, the fact that these plants are expected to contain only one functional receptor indicates that the presence of a single functional is sufficient to compensate for the absence of other receptors, i.e. there may be functional redundancy within the receptor group.

### Phenotypic Characterisation of T1 Plants

### Rattling Test

The rattling test used to screen the T0 plants for putative mutants was performed on ripening T1 pods from a side branch on PA and DPA plants as described above, i.e. the PA and DPA plants described in Table 6. Branches at a highly similar developmental stage on the PA and DPA plants were selected to ensure a suitable comparison could be made.

Both PA and DPA plants displayed rattling in the most mature pods. However, the onset of rattling was delayed in the DPA plants in comparison to the PA plants, indicating that DPA plants also were deficient in seed abscission.

As it is difficult to quantify the rattling phenotype, seed pods were opened to determine whether the absence or reduction of rattling in the DPA plants was caused by seed retention on the replum. However, as seed pods are easily twisted under attempts to open them along the dehiscence zone, the process of opening the pods resulted in seed detachment. Thus, although examples of seed retention were observed in both T0 and T1 DPA plants following opening of the seed pods, it was challenging to accurately quantify the seed retention effect. As such, an alternative test was devised.

### Shaking Test

Intensive shaking was used to test whether seed pods from DPA plants shed seeds less than seed pods from PA plants. The number of released seeds (FIG. 5) and percentage of closed, half-open and fully opened pods was assessed (FIG. 6). Comparison between PA and DPA plants demonstrates that fewer seeds were released from pods from DPA plants, and that pods from DPA plants were more resistant to destruction by shaking.

### Conclusions

The results of the rattling and shaking tests suggest that plants containing mutations that decrease or eliminate the function of all HAE and HLS2 mutants affect pod dehiscence as well as seed retention. Moreover, as mutant plants with increased seed retention (i.e. decreased seed abscission) were also deficient in petal abscission, the results support the notion that HAE and HSL2 control cell separation events generally in flowering plants. This is further supported by the observation that in the DPA plants cauline leaves sitting on the B. napus stem remain attached even when the plant is completely dry.

### Materials and Methods

Identification of B. rapa Genes

B. rapa orthologues of the genes encoding IDA and IDL peptides and HAE and HSL2 receptors were identified by using the A. thaliana proteins as queries in BLASTp and tBLASTn searches.

### Constructs

Constructs were generated using Gateway technology. Promoter fragments of the B. rapa genes BraAHSL2, BrIDA.a and BraAIDA.b of 2081, 1422 and 1415 bp respectively, were amplified with POR and introduced into pDONOR Zeo, and thereafter recombined into the GUS vector pMDC162. The BraA.HSL2.a (A09p010050), BraA.IDA.a (A07p042260) and Bra.IDA.b (A02p022480) genes with 2000 bp, 1020 and 1029 basepairs (bp) upstream the start codon and 80, 373 and 402 bp downstream of the stop codon, respectively, were likewise cloned in pDONOR Zeo and recombined into pMDC123. A. thaliana Col-0 plants were transformed using the floral dip method.

### Plant Material

Arabidopsis plants were cultivated in growth chambers at 22° C. for 8 h of dark and 16 h of light (100 μE·m-2·s-1).

Seeds of B. rapa var. R-o-18 wild type were sown in John Innes No. 2 compost and kept at 7° C. with no light for 3 days before being transferred into the glasshouse at 18° C. with 16 h light.

Seeds of B. napus were sown in John Innes No. 2 compost and kept at 7° C. with no light for 3 days before being transferred into the glasshouse at 18° C. with 16 h light.

### Rattling Test

Seed pods were harvested from side branches of test (DPA) and control (PA) plants displaying seed pods at a similar developmental stage. Seed pods were manually shaken near the ear and the sound of rattling (caused by seeds released from seed sticks inside the pods) was categorised as low or high.

### Shaking Tests

Seed pods were harvested from side branches of test (DPA) and control (PA) plants displaying seed pods at a similar developmental stage. Ten pods from each plant were subjected to manual shaking three times for 5 seconds in a 10 mm diameter bucket with three steel bullets (3×5 mm). The total number of released seeds after the final shake was determined. The number of closed, half-open and fully opened pods was scored after each 5 second shake.

1. A mutant or modified Brassica oilseed plant or part thereof comprising a heritable mutation in a HAESA (HAE) gene and in a HAESA-LIKE2 (HSL2) gene, wherein the mutant or modified plant is deficient in seed abscission in comparison to a corresponding unmutated or unmodified plant.

2. The plant or part of claim 1, wherein the plant comprises a heritable mutation in more than one HAE gene and/or more than one HSL2 gene.

3. The plant or part of claim 1 or 2, wherein the plant comprises a heritable mutation in all of the HAE genes and/or all of the HSL2 genes.

4. The plant or part of any one of claims 1 to 3, wherein the heritable mutations result in decreased expression of the HAE gene(s) and the HSL2 gene(s) in comparison to the expression in a corresponding unmutated or unmodified plant.

5. The plant or part of any one of claims 1 to 4, wherein the heritable mutations result in decreased activity of the proteins encoded by the HAE gene(s) and the HSL2 gene(s) in comparison to the activity in a corresponding unmutated or unmodified plant.

6. The plant or part of any one of claims 1 to 5, wherein the heritable mutations are frameshift mutations and/or result in a premature stop codon in the coding sequence relative to the corresponding unmutated gene, preferably wherein the heritable mutations are knock-out mutations.

7. The plant or part of any one of claims 1 to 6, wherein the Brassica oilseed plant is Brassica napus, Brassica rapa, Brassica nigra, or Brassica juncea.

8. The plant or part of any one of claims 1 to 7, wherein the HAE gene(s) comprise(s) a nucleotide sequence selected from any one of SEQ ID NOS: 1, 4, 13, 19, 22, or 31 or a nucleotide sequence with at least about 90% (e.g. at least about 95%, 96%, 97%, 98% or 99%) sequence identity to a nucleotide sequence selected from any one of SEQ ID NOs: 1, 4, 13, 19, 22, or 31 and/or encode a HAE polypeptide comprising an amino acid sequence selected from any one of SEQ ID NOs: 3, 6, 15, 21, 24, or 33 or a HAE polypeptide comprising an amino acid sequence with at least about 90% (e.g. at least about 95%, 96%, 97%, 98% or 99%) sequence identity to an amino acid sequence selected from any one of SEQ ID NOs: 3, 6, 15, 21, 24, or 33.

9. The plant or part of any one of claims 1 to 8, wherein the HSL2 gene(s) comprise(s) a nucleotide sequence selected from any one of SEQ ID NOS: 7, 10, 16, 25, 28, or 34 or a nucleotide sequence with at least about 90% (e.g. at least about 95%, 96%, 97%, 98% or 99%) sequence identity to a nucleotide sequence selected from any one of SEQ ID NOs: 7, 10, 16, 25, 28, or 34 and/or encode a HSL2 polypeptide comprising an amino acid sequence selected from any one of SEQ ID NOs: 9, 12, 18, 27, 30, or 36 or a HSL2 polypeptide comprising an amino acid sequence with at least about 90% (e.g. at least about 95%, 96%, 97%, 98% or 99%) sequence identity to an amino acid sequence selected from any one of SEQ ID NOs: 9, 12, 18, 27, 30, or 36.

10. The plant part of any one of claims 1 to 9, wherein the plant part is a seed pod or seed.

11. Seed of the mutant or modified Brassica oilseed plant of any one of claims 1 to 9, wherein the seed carries the heritable mutations as defined in any one of claims 1 to 9.

12. A method for producing a mutant or modified Brassica oilseed plant that is deficient in seed abscission in comparison to a corresponding unmutated or unmodified plant, the method comprising modifying a Brassica oilseed plant or cell to decrease the expression of a HAE gene and an HSL2 gene and/or to decrease the activity of the proteins encoded by said genes in comparison to the expression and/or activity in a corresponding unmutated or unmodified plant.

13. The method of claim 12, wherein the method comprises introducing a heritable mutation in a HAE gene and in an HSL2 gene in said plant or cell, wherein the heritable mutations result in decreased expression of the HAE gene and the HSL2 gene and/or decreased activity of the proteins encoded by said genes in comparison to the expression and activity in a corresponding unmutated or unmodified plant.

14. The method of claim 13, wherein the method comprises introducing a heritable mutation in all of the HAE genes and/or all of the HSL2 genes in said plant or cell, wherein the heritable mutations result in decreased expression of the HAE gene(s) and the HSL2 gene(s) and/or decreased activity of the proteins encoded by said genes in comparison to the expression and/or activity in a corresponding unmutated or unmodified plant.

15. The method of any one of claims 12 to 14, wherein the method comprises:
(i) genetically modifying a Brassica oilseed plant cell to: (1) introduce the heritable mutations defined in claim 13 or 14; or (2) introduce a transgene that results in the decrease in expression and/or activity; and
(ii) regenerating a plant comprising the heritable mutations or transgene from the plant cell produced in (i).

16. The method of claim 15, wherein the step of genetically modifying a Brassica oilseed plant cell uses CRISPR/Cas gene editing.

17. The method of claim 15 or 16 comprising a step of propagating the regenerated plant and obtaining seed comprising the heritable mutations or transgene.

18. The method of any one of claims 12 to 17, wherein:
(i) the heritable mutations are as defined in claim 6;
(ii) the Brassica oilseed plant is as defined in claim 7;
(iii) the HAE gene(s) is (are) as defined in claim 8; and/or
(iv) the HSL2 gene(s) is (are) as defined in claim 9.

19. A modified Brassica oilseed plant obtainable from the method of any one of claims 12 to 18, wherein the plant comprises a genetic modification (e.g. transgene) that results in decreased expression of one or more (preferably all) HAE genes and one or more (preferably all) HSL2 genes and/or decreased activity of the proteins encoded by said genes in comparison to the expression and/or activity in a corresponding unmodified plant.

20. Seed obtainable from the modified Brassica oilseed plant of claim 19, wherein the seed comprises a genetic modification (e.g. transgene) that results in decreased expression of one or more (preferably all) HAE genes and one or more (preferably all) HSL2 genes and/or decreased activity of the proteins encoded by said genes in a plant grown from the seed in comparison to the expression and/or activity in a corresponding plant grown from unmodified seed.

21. Use of:
(i) a plant or part thereof according to any one of claim 1 to 10 or 19;
(ii) seed according to claim 11 or 20; or
(iii) a plant produced by the method of any one of claims 12 to 18,
to:
(a) breed a plant;
(b) grow a crop; or
(c) produce seed.

22. Use of:
(i) a plant or part thereof according to any one of claim 1 to 10 or 19;
(ii) seed according to claim 11 or 20; or
(iii) a plant produced by the method of any one of claims 12 to 18,
to obtain oil (e.g. edible oil) and/or one or more by-products from the oil extraction and/or processing procedure.

23. A method of obtaining the seed of claim 11 or 20 comprising:
(a) providing:
(i) a plant or part thereof (e.g. seed pod) according to any one of claim 1 to 10 or 19; or
(ii) a plant produced by the method of any one of claims 12 to 18; and
(b) harvesting and/or separating seed from the plant or part thereof of (a).

24. A method of obtaining oil comprising:
(a) providing seed according to claim 11 or 20 or seed obtained from the method of claim 23; and
(b) extracting oil from the seed of (a).

25. The method of claim 24 further comprising processing the oil (e.g. crude oil) extracted from the seed to produce a processed oil or oil product and/or one or more by-products.
